(19) United States
(12) Patent Application Publication
(10) Pub. No.: US 2008/0215039 A1
(43) Pub. Date: Sep. 4, 2008

(54) Method and Apparatus for Inhibiting Pain Signals During Vacuum-Assisted Medical Treatments of the Skin

(75) Inventors: Michael Slatkine, Herzliya (IL); Raphael Shavit, Tel Aviv (IL)

Correspondence Address: KEVIN D. MCCARTHY;ROACH BROWN MCCARTHY & GRUBER, P.C., 424 MAIN STREET, 1920 LIBERTY BUILDING, BUFFALO, NY, 14202, US

(73) Assignee: Inolase 2002 Ltd., Netanya (IL)

(21) Appl. No.: 11/997,428

(22) PCT Filed: Aug. 3, 2006
(86) PCT No.: PCT/IL2006/000897

§ 371 Date: Feb. 11, 2008

(30) Foreign Application Priority Data
Aug. 4, 2005 (IL) 170132
Apr. 6, 2006 (IL) 174847
Apr. 11, 2006 (US) 11/401674
May 8, 2006 (IL) 175486

(51) Int. Cl. A61B 18/18; A61B 19/00; A61N 5/06

(52) U.S. Cl. 606/9; 601/6

(57) ABSTRACT

An apparatus adapted to inhibit pain signals generated by pain receptors in the skin during a skin related medical treatment such as an injection. An evacuation chamber is provided with an essentially rigid interface element through which a medical treatment can be administered to a selected skin region, one or more walls which are placeable on, or in the vicinity of, the skin region, an interior defined by the walls and by the interface element, and an opening at the bottom of the interior which is sealable by the skin region. A device generates a vacuum within the evacuation chamber interior to a level suitable for drawing the skin region through the opening towards, and in a compressing relation against, the interface element, to inhibit the transmission of a pain signal generated by pain receptors located within the skin region.

### FIELD OF INVENTION

The current invention is related to an apparatus adapted to inhibit the sensation of pain during skin treatments in general, and particularly during needle injections, ultrasonic treatments, ultrasonic disruption of tissue, hair removal with a hand held implement, and light based skin treatments.

### BACKGROUND OF THE INVENTION

Many medical treatments are accompanied by a pain sensation. The pain sensed within a skin region is generated by pain receptors in the skin. According to the Gate Theory of Afferent Inhibition described in, for example, “The Physiology Coloring Book,” W. Kapit et al, Harper Collins Publishers (1987), pages 88-89, the pressure sensed by large, fast-conducting tactile nerves, such as by rubbing the skin, limits the transmission gates in the dorsal horn, excludes access for the weaker pain signal, and therefore inhibits the pain signal transmission by pain nerves in the spinal cord.

The treatment of skin is often very painful and may necessitate the utilization of analgesic topical creams such as EMLA cream produced by AstraZeneca Inc., Canada, or even of anesthetic injections to inhibit the pain. Such pain inhibiting procedures are risky and also increase the total duration of the treatment.

In resent years, ultrasonic treatments of lesions located under the skin have been proposed and performed. By directing ultrasonic waves and the associated mechanical force in the form of vibrations at the lesions, a localized increase in temperature sufficient to treat the lesion is noticeable, This localized increase in temperature also causes a pain sensation. In one ultrasonic treatment, subcutaneous lesions are disrupted with focused ultrasonic waves which generate cavitation in the focal zone, in order to selectively destroy tissue. Examples of ultrasonic cavitation tissue disruptors are a device produced by Ultrashape Ltd., Israel for the reduction of fat and cellulite, which is described in US 2003/0083536 and US 2005/0261584, and a device for the treatment of malignant tissue under the skin produced by GE Medical Systems, USA. The ultrasonic frequency of most treatments is in the 1-10 MHz range, and the focused ultrasonic treatments are generally very painful.

Ultrasonic energy may be used for hair removal, as described in U.S. Pat. No. 6,544,259. Hair removal by means of an ultrasonic device is generally very painful.

Another disruptive treatment which is liable to cause pain is the vibration of hair at a frequency equal to, or slightly greater than, sonic frequency. Disruptive elements are adapted to induce cavitation and disrupt tissue. Cavitation can also be induced under the skin by means of an ultrasonic aspirator.

Another medical treatment that is generally painful is the subcutaneous injection of a medication by means of a needle, causing much apprehension to patients prior to the injection.

U.S. Pat. No. 6,132,392 discloses a pain relieving device for selectively applying three or more pain relieving modalities to an upper body region of a person, including acupressure and at least two other modalities selected from the group of vibration, massage, heat, traction and electric stimulation. The deficiencies of this device with respect to pain inhibition include a low pain reduction, lack of repeatability, and considerable inconvenience to the patient.

US 2002/0013602 discloses a method for reducing the pain associated with an injection or minor surgical procedure at a site on the skin of a patient by urging a skin engaging surface of a pressure member against the skin proximate the site, thereby stimulating the large diameter afferent sensory nerve fibers in the skin proximate the site and at least partially blocking pain signals from the small diameter afferent pain nerve fibers in the skin proximate the site. Some of the disadvantages of this method include the difficulty and inconvenience to apply pressure on skin which is in close proximity to bones, the inability to achieve treatment repeatability, and the difficulty to maintain a constant level of pressure on the skin. Also, while applying external pressure on a skin surface, the pressure receptors are pressed before any pressure is exerted on a deeper neuron extending from the pain receptor. Furthermore, the reactive force applied by bones underlying the pressed skin increases the skin squeezing effect, affecting the compression of blood vessels and of nerves.

It would be desirable to provide a method for alleviating pain during injections or ultrasonic treatments which is more easily carried out and has a greater repeatability than that of the prior art.

US 2004/0254599 discloses a method and apparatus for reducing the perceived pain resulting from the puncturing of skin at a puncture site by generating a sensory distraction, such as vibration, an acoustical signal or an electrical stimulation. One deficiency of this method is that the sensory distraction is generated by means of a relatively complicated mechanism or electronic device. Another deficiency is that a pain inhibiting pressure signal is simulated, and a limited number of pressure sensors, if any, are involved in the pain inhibiting process. A pain signal that is not inhibited by a compressed pressure receptor will therefore be transmitted.

Some prior art devices reduce the level of generated pain by limiting the depth of needle penetration into the skin. WO 2004/004803 discloses an intradermal delivery device comprising a housing including a base deeming a needle aperture, and a skin-engaging surface extending about a periphery of the needle aperture. A syringe of the intradermal delivery device includes a syringe body coupled to the housing and a plunger slidably received within the syringe body. A needle is coupled in fluid communication with the syringe body, and is movable through the needle aperture to penetrate the skin and inject a substance contained within the syringe body therein. A evacuation chamber of the intradermal delivery device is coupled in fluid communication with the base for drawing a vacuum within the base and, in turn, releasably securing the skin-engaging surface to the skin and forming a substantially planar needle penetration region on the skin. The intradermal delivery device further includes at least one stop surface fixed relative to at least a portion of the skin-engaging surface to define a predetermined distance therebetween, and adapted to cooperate with the needle to limit an insertion of the needle into the needle penetration region of the skin. Such a device is incapable of reducing pain when the medical treatment requires a deeper injection depth. Also a downward force is applied to the skin surface.

JP 2001-212231 discloses a device capable of reducing the sensed pain at the time of penetration of a syringe needle. As the housing portion of the device is pressed onto the epidermis of a patient, the air within a variable volume chamber is released. When the syringe needle is introduced into the skin through a suitable aperture of the device, the force applied onto the epidermis as a result of the pressure differential between atmospheric air and that of the chamber helps to disperse the sensed pain during penetration of the syringe needle into the skin. Only the walls of the variable volume chamber having a very small projected surface area contact the drawn skin, and therefore the pain reduction that may be realized with this device is very limited. Also, the vacuum level that is generated by downwardly pressing the housing portion onto the epidermis is very low, and is not sufficient to reduce the sensed pain to a significant extent.

JP 2005-087520 discloses a liquid medicine injector that causes a reduced sensation of pain as a skin region is punctured. A plurality of hollow needles communicate with a liquid medicine container and project outwardly therefrom. A leaf spring provides the driving force for percutaneously injecting the liquid medicine via a needle, and a suction port sucks the air in a recessed part of the container, from which the needles project. In order to reduce pain, a dedicated injector that is compatible with the configuration of the apparatus must be employed. Also, the Gate Theory of Afferent Inhibition is not mentioned in this publication, and therefore a threshold size and threshold vacuum level for achieving pain reduction is not suggested. Furthermore, the sole purpose of generating the vacuum is not to reduce pain, but also to prevent the needle from coming off from the skin in order to ensure precise penetration into the skin at a desired location and depth.

Prior art very high intensity, short duration pulsed light systems which operate in the visible part of the spectrum, such as flashlamps or intense pulsed lasers are currently used in aesthetic treatments by one of two known ways: a) Applying the light to the skin without applying any pressure on the treatment zone, so as not to interfere with the natural absorption properties of skin; and b) Applying pressure onto the skin by means of the exit window of the treatment device in contact with the skin, thereby expelling blood from the light path within the skin and enabling better transmission of the light to a skin target in cases where the spectral lines of the treatment light source match absorption lines of the blood.

The major applications of intense pulsed light or intense pulsed laser systems are hair removal, coagulation of blood vessels for e.g. port wine stains, telangectasia, spider veins and leg veins, multiple heating of blood vessels for e.g. rosacea, treatment of pigmented skin such as erasure of black stains and sun stains or tattoo removal, and removal of fine wrinkles by heating the tissue around the wrinkles, normally referred to as photorejuvenation.

U.S. Pat. Nos. 5,226,907, 5,059,192, 5,879,346, 5,066,293, 4,976,709, 6,120,497, 6,120,497, 5,626,631, 5,344,418, 5,885,773, 5,964,749, 6,214,034 and 6,273,884 describe various laser and non-coherent intense pulsed light systems. These prior art light systems are not intended to increase the natural absorption of the skin. These prior art light systems are also not intended to block pain transmission during treatments.

Applying a vacuum to the skin is a known prior art procedure, e.g. for the treatment of cellulites, which complements massaging the skin. Such a procedure produces a flow of lymphatic fluids so that toxic substances may be released from the tissue. As the vacuum is applied, a skin fold is formed. The skin fold is raised above the surrounding skin surface, and the movement of a handheld suction device across the raised skin performs the massage. The suction device is moved in a specific direction relative to the lymphatic vessels, to allow lymphatic fluids to flow in their natural flow direction. The lymphatic valve in each lymphatic vessel prevents the flow of lymphatic fluid in the opposite direction, if the suction device were moved incorrectly. Liquids generally accumulate if movement is not imparted to the raised skin. The massage, which is generally carried out by means of motorized or hand driven wheels or balls, draws lymphatic fluids from cellulite in the adipose subcutanous region and other deep skin areas, the depth being approximately 5-10 mm below the dermis.

U.S. Pat. No. 5,961,475 discloses a massaging device with which negative pressure is applied to the skin together during massaging. A similar massaging device which incorporates a radio frequency (RF) source for the improvement of lymphatic flow by slightly heating the adipose tissue is described in U.S. Pat. No. 6,662,054. Some massaging systems, such as those produced by Deka and Cynosure, add a low power, continuous working (CW) light source of approximately 0.1-2 W/cm², in order to provide deep heating of the adipose tissue by approximately 1-3° C. degrees and to enhance lymphatic circulation. The light sources associated with vacuum lymphatic massage devices are incapable of inducing blood vessel coagulation due to their low power. Also, prior art vacuum lymphatic massage devices are adapted to induce skin protrusion or to produce a skin fold by applying a vacuum.

Selective treatment of blood vessels by absorption of intense pulsed laser radiation is possible with Dye lasers operating at 585 nm, as well as with other types of lasers. Photorejuvenation has also been performed with Diode lasers in the near infrared spectral band of 800-980 nm and with Nd:YAG lasers having a frequency of approximately 1064 nm with limited success. The light emitted by such lasers is not well absorbed by tiny blood vessels or by the adjoining liquid. Broad band non-coherent intense pulsed light systems are also utilized for photorejuvenation with some success, although requiring more than 10 repeated treatments. The heat which is absorbed by the blood vessels, as a result of the light emitted by the intense short pulse devices, is transferred to adjacent collagen bundles.

The absorption of pulsed Diode and Nd:YAG laser beams by blood vessels is lower than the absorption of pulsed Dye laser beam. In order to compensate for limited photorejuvenation with red and infrared intense pulsed light and laser systems, a very high energy density as high as 30-60 J/cm² needs to be generated. At such an energy density, the melanin-rich epidermis, particularly in dark skin, is damaged if not chilled. A method to reduce the energy density of intense pulsed lasers or non-coherent intense pulsed light sources which operate in the visible or the near infrared regions of the spectrum will therefore be beneficial.

Pulsed dye lasers operating in the yellow spectral band of approximately 585-600 nm, which is much better absorbed by blood vessels, are also utilized for the smoothing of fine wrinkles. The energy density of light emitted by Dye lasers, which is approximately 3-5 J/cm², is much lower than that of light emitted by other lasers. However, the pulse durations of light emitted by Dye lasers are very short, close to 1 microsecond, and therefore risk the epidermis in darker skin. Treatments of wrinkles with Dye lasers are slow, due to the low concentration of absorbing blood vessels, as manifested by the yellow or white color of treated skin, rather than red or pink characteristic of skin having a high concentration of blood vessels. Due to the low energy density of light emitted by Dye lasers, as many as 10 treatments may be necessary. A method to reduce the energy density of light generated by Dye lasers, or to reduce the number of required treatments at currently used energy density levels, for the treatment of fine wrinkles, would be beneficial.

Pulsed Dye lasers operating at 585 nm are also utilized for the treatment of vascular lesions such as port wine stains or telangectasia or for the treatment of spider veins. The energy density of the emitted light is approximately 10-15 J/cm², and is liable to cause a burn while creating the necessary purpura. A method to reduce the energy density of light emitted by Dye lasers for the treatment of vascular lesions would be highly beneficial.

Hair removal has been achieved by inducing the absorption of infrared light, which is not well absorbed by melanin present in hair strands, impinging on blood vessels. More specifically, absorption of infrared light by blood vessels at the distal end of hair follicles contributes to the process of hair removal. High intensity pulsed Nd:YAG lasers, such as those produced by Altus, Deka, and Iridex, which emit light having an energy density of more than 50 J/cm², are used for hair removal. The light penetration is deep, and is often greater than 6 millimeters. Some intense pulsed light or pulsed laser systems, such as that produced by Syneron, used for hair removal or photorejuvenation also employ an RF source for further absorption of energy within the skin.

The evacuation of smoke or vapor, which is produced following the impingement of monochromoatic light on a skin target, from the gap between the distal end window of a laser system and the skin target, is carried out in conjunction with prior art ablative laser systems such as Co₂, Erbium or Excimer laser systems. The produced smoke or vapor is usually purged by the introduction of external fresh air at greater than atmospheric pressure.

Coagulative lasers such as pulsed dye lasers or pulsed Nd:YAG lasers, which treat vascular lesions under the skin surface without ablating the skin surface, are generally not provided with an evacuation chamber which produces subatmospheric pressure over a skin target.

Some prior art intense pulsed laser systems, which operate in the visible and near infrared region of the spectrum and treat lesions under the skin surface, e.g. vascular lesions, with pulsed dye laser systems or pulsed Nd:YAG lasers, employ a skin chilling system. Humidity generally condenses on the distal window, due to the use of a skin chilling system. The humidity is not caused by the skin treatment, but rather by the low temperature of the distal window. It would be advantageous to evacuate the condensed vapors from the distal window of the laser system prior to the next firing of the laser.

U.S. Pat. Nos. 5,595,568 and 5,735,844 describe a coherent laser system for hair removal whereby pressure is applied to the skin by a transparent contact device in contact therewith, in order to expel blood present in blood vessels from a treatment zone. In this approach blood absorption decreases in order to increase subcutaneous light penetration.

U.S. Pat. Nos. 5,630,811 and 5,853,407 also describe a coherent laser system for hair removal which restricts local blood flow, in order to reduce damage to the skin tissue surrounding the hairs. Local tissue structures are flattened by applying positive pressure or negative pressure to the skin. The treatment beam is limited to only 5 mm. The treatment beam is not suitable for a larger treatment spot per pulse of approximately 40 mm. Also, the pressure level which has to be applied is not recited, although different pressures levels will lead to different effects. Some of these effects cannot be achieved with a beam diameter of 5 mm or less, as will be described hereinafter. Blood expulsion resulting from the pressing of skin is not uniform and is not instantaneous for such large treatment spots, and therefore blood may remain in the skin tissue after the laser beam has been fired. Also, a large-diameter treatment device may not be easily repositioned to another treatment site, due to the relatively high lifting force needed when negative pressure is applied to the skin. Furthermore, this laser system does not provide any means for preventing gel obstruction when negative pressure is applied to the skin. Although applying a flattening positive pressure or negative pressure to a small-diameter treatment area enhances hair removal, the treatment of vascular lesions is not improved since fewer blood vessels are present within the treatment area due to the blood expulsion. A need therefore exists for a vacuum-assisted device that can alternatively reduce or increase the blood volume fraction within a skin target.

US 2002/0128635 discloses a head for applying light energy to a selected depth in a scattering medium having an outer layer in physical and thermal contact with the head. The head includes a thermally conductive block having an energy emitting surface and at least one laser diode mounted in the block adjacent the energy emitting surface. At the bottom of the block is attached a transparent element having a high reflectivity mask with slits, for optimizing retroreflection of scattered energy from the skin. In one embodiment, the block is formed with a recess therein, into which a vacuum draws the skin. The head is not easily repositioned to another treatment site in order to treat a large-area skin surface, due to the relatively high lifting force needed when the vacuum is applied to the skin. Furthermore, means are not provided for preventing gel obstruction when a vacuum is applied to the skin.

The light-based non-ablative treatment of hair or of vascular lesions is often very painful, particularly during the treatment of dark and thick unwanted hairs which may appear in a bikini line, on the legs, or on the back. A pain sensation is felt with almost all types of light based devices for hair removal, including intense pulsed light sources and lasers.

The aforementioned prior art efforts to expel blood vessels help in some cases to avoid unnecessary damage to skin structures which are not intended to be treated, such as unnecessary coagulation of blood vessels during a hair removal treatment, while increasing hair removal efficacy. The reduction in damage to skin structures does not alleviate the immediate pain sensed during a treatment, but rather, the expulsion of blood causes a higher exposure of the hair shaft to a treatment pulse of light, resulting in a higher hair follicle temperature and a correspondingly higher level of acute pain due to excessive heating of the nerves which surround the hair shafts. Furthermore, the expelling of blood from one skin area increases the fractional blood volume in adjacent areas, causing a risk of thermal damage if the treatment light diffuses to the adjacent blood rich zone. It is well known to light-based hair removal practitioners that acute pain is felt during the treatment when hairy areas, particularly characterized by dark thick hair, are impinged by the treatment beam, whereas firing the light beam on a hairless area is substantially painless. It can therefore be concluded that the pain which is sensed during a hair removal treatment is generated by nerves surrounding the hair shafts, and not by nerves distributed in other areas of the skin. There is therefore a need for an improved apparatus for pain reduction without having to reduce the treatment energy density.

During light-based skin treatments, pain nerves in the vicinity of the epidermis and adjacent to hair follicles sense a relatively high increase in temperature of the hair follicle, often greater than 70° C. If not inhibited, the pain nerves transmit a pain signal to the brain via the spinal cord. Due to sensed pain, the treatment time is considerably increased.

Two types of a pain sensation caused by light-based aesthetic treatments are recognizable: immediate sharp pain and long term milder pain. The immediate sharp pain is felt during each treatment pulse and increases to an intolerable sensation after a few shots, necessitating a patient to rest during a long delay before continuing the treatment. The treatment rate, particularly for large areas such as on the legs, is therefore considerably reduced. Depending on his pain tolerance, the patient may even decide not to continue the treatment. The sharp pain is caused by the exposure of treatment light to nerve endings located in the epidermis and dermis, by sensory receptors of hair shafts located deep in the dermis, or by the stimulation of nerves surrounding the hair bulbs as the hair shafts are being heated during the treatment, often at a temperature of approximately 70° C.

The less acute, long term milder pain is caused by the accumulative increase of skin temperature following treatment, e.g. during a period ranging from 10 minutes to a day after treatment, which is approximately 3 to 5° C. above body temperature. The increase in skin temperature may induce redness and edema, causing pain, depending on the hair density and the fractional blood volume within the adjoining tissue. The application of a cold gauze immediately after the treatment usually helps to avoid the post-treatment pain.

The most common prior art method for alleviating or preventing the immediate sharp pain caused by the non-ablative treatment of hair or of vascular lesions with intense pulsed light is the application of EMLA cream produced by AstraZeneca Canada Inc. Such cream is a topical anesthetic applied to the skin approximately 30-60 minutes before a treatment, which numbs the skin and decreases the sensation of pain. This prior art method is generally impractical due to the long and inconvenient waiting time between the application of the EMLA cream and the treatment. Since health professionals prefer to maximize the number of patients to be treated during a given time period, the health clinic must provide a large waiting room for those patients that are waiting to be treated by intense pulsed light following the application of the EMLA cream.

Pain caused by the non-ablative treatment of hair or of vascular lesions may also be alleviated or prevented by reducing the energy density of the intense pulsed light. Energy density reduction, however, compromises the treatment quality, and therefore is an unacceptable solution, particularly due the relatively high cost of treatment.

U.S. Pat. Nos. 6,264,649 and 6,530,920 disclose a cooling head for a skin treatment laser and a method to reduce or eliminate pain during laser ablative treatments of the skin by cooling the skin surrounding the treatment area. The pain is associated with the ablation or burning of a skin surface during skin resurfacing. An extraction port of the cooling head enables removal of debris material, such as smoke produced by the skin treatment laser, from the treatment area and for connection to a vacuum source. Evacuated vapor such as smoke is replaced by fresh and clean air.

With respect to prior art smoke evacuation devices, a significant subatmospheric pressure is generally not generated over a skin surface due to the introduction of fresh atmospheric pressure air. If subatmospheric pressure were maintained over a skin surface, the treatment handpiece would be prevented from being lifted and displaced from one skin site to another during the treatment process. Additionally, prior art smoke evacuation devices are not associated with non-ablative lasers, such as a long-pulse Nd:YAG laser, which treat tissue only under the skin surface and do not produce smoke resulting from the vaporization of the skin surface. Furthermore, the application of heat releasing gel onto a skin target is not conducive for the ablation of a skin surface or for the subsequent evacuation of debris material since the gel forms a barrier between the skin surface and the surrounding air.

Current laser and IPL skin treatment systems utilize chilling means. However, pain is still noticeable.

A need therefore exists for alleviating the resulting pain caused by the treatment of unwanted hair, unwanted wrinkles or vascular lesions by intense pulsed light or intense pulsed laser systems, without reducing the light source intensity, without applying a topical anesthetic, and without using a chiller as means to reduce pain.

It is an object of the present invention to provide a method and apparatus for inhibiting the resulting pain which is usually sensed during a skin-related medical treatment, such as an ultrasonic treatment of the skin or during an injection into the skin, without use of an analgesic topical cream or anesthetic injection.

It is an object of the present invention to provide a method and apparatus for achieving a large level of pain reduction during a skin-related medical treatment.

It is an additional object of the present invention to provide a method and apparatus for increasing the level of pain reduction during a skin-related medical treatment with respect to prior art vacuum-assisted pain inhibiting apparatus.

It is an additional object of the present invention to provide a method and apparatus for performing painless skin-related medical treatments of high repeatability.

It is an additional object of the present invention to provide apparatus having a threshold size and generating a threshold vacuum level, in order to achieve a desired level of pain reduction.

It is an additional object of the present invention to provide apparatus that is suitable for inhibiting the transmission of pain signals in the dorsal horn by generating pressure signals in a sufficiently high number of pressure receptors in the skin.

It is an additional object of the present invention to provide a method and apparatus for performing painless injections with any commercially available injector.

It is yet an additional object of the present invention to provide a method and apparatus for the treatment of subcutaneous lesions, such as vascular lesions, by a non-ablative, high intensity pulsed laser or light system operating at wavelengths shorter than 1800 nm which does not damage the surface of the skin or the epidermis.

It is yet an additional object of the present invention to provide a method and apparatus for controlling the depth of subcutaneous light absorption.

It is yet an additional object of the present invention to provide a method and apparatus for increasing the absorption of light which impinges a skin target by increasing the concentration of blood vessels thereat.

It is yet an additional object of the present invention to provide a method and apparatus by which the energy density level of intense pulsed light that is suitable for hair removal, fine wrinkle removal, including removal of wrinkles around the eyes and in the vicinity of the hands or the neck, and the treatment of port wine stain or rosacea may be reduced relative to that of the prior art.

It is yet an additional object of the present invention to provide a method and apparatus by which the number of required treatments for hair removal, fine wrinkle removal, including removal of wrinkles around the eyes and in the vicinity of the hands or the neck, and the treatment of port wine stain or rosacea at currently used energy density levels may be reduced relative to that of the prior art.

It is yet an additional object of the present invention to provide a method and apparatus for repeated evacuation, prior to the firing of a subsequent light pulse, of vapors which condense on the distal window due to the chilling of laser treated skin.

It is yet an additional object of the present invention to provide a method and apparatus for alleviating the resulting pain caused by the treatment of unwanted hair, unwanted wrinkles or vascular lesions by intense pulsed light or intense pulsed laser systems, without reducing the light source intensity, without applying a topical anesthetic, and without relying on skin chilling for pain reduction.

It is yet an additional object of the present invention to provide a method and apparatus for speedy repositioning of a vacuum-assisted, non-ablative light-based treatment handpiece from one site to another.

It is yet an additional object of the present invention to provide a method and apparatus for a vacuum-assisted, light-based skin treatment which is conducive for the application of a heat releasing gel onto a skin surface, without resulting in obstruction of vacuum generating apparatus.

It is yet an additional object of the present invention to provide an apparatus for vacuum-assisted, light-based treatment which can be held by one hand while a light treatment handpiece is held by the other hand.

### SUMMARY OF THE INVENTION

The present invention provides an apparatus which inhibits pain signals generated by pain receptors in the skin during a skin related medical treatment from being transmitted to the brain by inducing a controlled compression of a skin region, comprising:
a) an evacuation chamber comprising an essentially rigid interface element through which a medical treatment can be administered to a selected skin region, one or more walls which are placeable on, or in the vicinity of, said skin region, an interior defined by at least said one or more walls and by said interface element, and an opening at the bottom of said interior which is sealable by said skin region;
b) means for generating a vacuum within said evacuation chamber interior, the level of the applied vacuum suitable for drawing said skin region through said opening towards, and in a compressing relation against, said interface element, whereby to inhibit the transmission of a pain signal generated by pain receptors located within said skin region; and
c) means for administering a skin related medical treatment, said administering means adapted to pass through said interface element and to be directed to said compressed and pain inhibiting skin region.

As referred to herein, the “interface element” is an element through the thickness of which the administering means can pass, yet is suitable for sufficiently secluding the evacuation chamber interior from the evacuation chamber exterior in order to achieve said vacuum level being suitable for inhibiting the transmission of pain signals.

As referred to herein, a “medical treatment” is a procedure administered to a living subject to improve a pathological disorder, to improve the appearance of a skin region, to effect a change in tissue, to introduce beneficial material into the body, and a dental or oral procedure such as making a small incision in the gums.

The administering means may be an injection needle, and the corresponding interface element is puncturable thereby or is provided with a plurality of apertures through each of which the injection needle may introduced.

The administering means may also be electromagnetic energy, such as ultrasonic waves, laser light, and IPL light, and the corresponding interface element is transparent or translucent to said electromagnetic energy.

As referred to herein, a “vacuum level” is the absolute pressure below atmospheric pressure. A vacuum level of 500 mmHg is therefore a pressure of 500 mmHg below atmospheric pressure. When a vacuum level is referred to as being greater than a given value, e.g. greater than 400 mmHg, the pressure within the evacuation chamber interior is an absolute pressure of a value below atmospheric pressure greater than said given value.

Preferably, the surface area of the interface element is greater than a threshold surface area, e.g. at least 100 mm², which is suitable for inhibiting the transmission of a pain signal generated by pain receptors located within the skin region.

Preferably, the vacuum level within the evacuation chamber is greater than a threshold vacuum level, e.g. at least 150 mmHg and preferably at least 400 mmHg, which is suitable for inhibiting the transmission of a pain signal generated by pain receptors located within the skin region.

In one aspect, the vacuum generating means comprises a vacuum pump, such as a dual air-gel vacuum pump, in fluid communication with the evacuation chamber.

In one aspect, the vacuum generating means is a vacuum source, such as a pre-evacuated container, in fluid communication with the evacuation chamber and means for isolating said vacuum source from the evacuation chamber interior. As referred to herein, a “vacuum source” is a member which is in communication with the evacuation chamber and is subjected to a sufficiently high vacuum to draw the skin region in compressing fashion against the interface element.

The volume of the vacuum source is preferably at least twice the volume of the evacuation chamber.

The isolation means may be a valve or a breakable stop, and may be openable by control means. The control means is preferably a controller and a skin contact detector in communication with said controller for sensing the placement of the evacuation chamber onto the skin region, said controller adapted to generate a signal for opening the isolation means following placement of the evacuation chamber onto the skin region.

In one aspect, the pre-evacuated container is integrally connected to the evacuation chamber.

A control means for synchronizing the activation of the vacuum generating means is preferably employed.

In one aspect, the control means is a mechanical control means, such as a pin placeable on the skin region, the pointed end of said pin adapted to pierce a membrane stretched across the interior of a conduit extending between the pre-evacuated container and the evacuation chamber once the evacuation chamber is placed on the skin region.

The control means may further comprise a valve in communication with both the conduit and surrounding ambient air, said valve being openable whereby to release the vacuum by kinematic means a predetermined period of time following placement of the evacuation chamber on the skin region.

In one aspect, the control means comprises a controller and a skin contact detector in communication with said controller for sensing the placement of the evacuation chamber onto the skin region, said controller adapted to generate a first signal for activating the vacuum generating means following placement of the evacuation chamber onto the skin region and to generate a second signal for deactivating the vacuum generating means a predetermined duration, e.g. no longer than approximately 6 seconds following generation of said first signal. The skin contact detector may be an opto-coupler or a microswitch.

The control means may further comprise a pressure sensor in fluid communication with the interior of the evacuation chamber and in electrical communication with the controller, the controller being further adapted to control the operation of the vacuum generating means so that a predetermined pain inhibiting vacuum level ranging between 400 mmHg and 1 atmosphere will be generated within the evacuation chamber.

In one aspect, the vacuum generating means comprises at least one control valve, the controller being suitable for delivering air through said at least one control valve in order to increase the pressure in the evacuation chamber to atmospheric pressure following the generation of the second signal, to allow for effortless repositioning of the evacuation chamber to a second skin region, the control means being selected from the group of electronic means, pneumatic means, electrical means, and optical means.

In one aspect, the interface element is pre-compressed.

In one aspect, the interface element is planar and may be retained in a cover element attached to one or more sidewalls.

In one aspect, the interface element is curvilinear and may be retained by one or more sidewalls.

The apparatus is suitable for the painless administration of medical treatments selected from the group of ultrasonic-based hair removal, ultrasonic-based collagen tightening, ultrasonic-based blood vessel sealing, ultrasonic-based treatment of-fatty or cellulite tissue, injections for vaccines, injections for the administration of drugs, injection of collagen, mesotherapy, removal of hair with a hand held implement, needle epilation, injection of collagen within the epidermis, tattoo removal, and the treatment of pigmented lesions.

In one embodiment, the means for administering the medical treatment is an injection needle.

In one aspect, the interface element is puncturable, and may be a subdivided interface element.

In one aspect, the interface element is an apertured interface element, an injection needle being introducible through each of said apertures. The total area of the apertures is less than 20% of the total area of the interface element.

In one aspect, the apertures are covered by a shield element. The shield element may be placed directly on top of the interface element, or alternatively, a seal element may be interposed between the shield element and the interface element. Marks corresponding to the location of the apertures are preferably indicated on the upper face of the shield.

In one aspect, the apparatus further comprises means for maintaining the vacuum within the evacuation chamber following termination of the vacuum generating means.

In one aspect, the vacuum maintaining means comprises a plurality of rims connected to the underside of the apertured interface element, each of said rims encircling a corresponding aperture formed in the interface element and adapted to produce a volume of negative pressure within the evacuation chamber for drawing the skin region in compressing relation against the interface element following termination of the vacuum generating means, said volume being enclosed by the interface element, rim, and drawn skin region or being enclosed by the interface element, evacuation chamber sidewall, and drawn skin region,

In one aspect, the apparatus further comprises means for guiding the injection needle through an aperture to the drawn skin region. In one aspect, the apparatus further comprises means for administering a plurality of injection needles, such as a bar for holding a plurality of needle applicators therebelow and a guide track substantially perpendicular to said bar.

In one aspect, an evacuation chamber sidewall is an interface element.

In one aspect, the evacuation chamber is configured as a slit defined by elongated, planar sidewalls and by a rigid upper surface extending between, and having a considerably shorter length than, said sidewalls.

In one aspect, the apparatus further comprises a vibrator kinematically connected to the interface element.

In one embodiment, the means for administering the medical treatment is a beam of ultrasonic waves and the interface element is made of a material which is transparent to ultrasonic waves. The ultrasonic waves preferably have a frequency ranging from 1 to 10 MHz and are generated by means of an ultrasonic transducer.

In one embodiment, an apparatus is provided for alleviating or preventing pain caused by a treatment with electromagnetic energy of a targeted skin structure, comprising:

a) an element subjected to a generated vacuum therebelow, the level of the generated vacuum being sufficiently high to draw a skin target underlying said element towards, and in a compressing relation against, said element, whereby to alleviate or prevent the transmission of a pain signal generated by pain receptors located within said skin target; and

b) a pulsed source of electromagnetic energy for generating waves that are transmitted through said element and that are suitable for treating a skin disorder within said skin target.

In one aspect, the electromagnetic energy is laser or IPL light having a wavelength ranging from 400 to 1800 nm.

In one aspect, the element which is subjected to a generated vacuum therebelow is an interface element of an evacuation chamber, said evacuation chamber further comprising one or more walls which are placeable on, or in the vicinity of, said skin region, an interior defined by at least said one or more walls and by said interface element, and an opening at the bottom of said interior which is sealable by the skin target.

In one aspect, each wall of the evacuation chamber is puncturable, a darkened needle capable of piercing a wall of the evacuation chamber, attracting the optical energy of the light, and thermally damaging the surrounding skin structure.

In one aspect, the light has an energy density ranging from 10 to 100 J/cm² and a pulse duration ranging from 10 to 300 millisec.

In one aspect, the apparatus further comprises gliding apparatus for displacing a light source distal end over the interface element at a speed ranging from 0.3 to 40 cm/sec.

In one aspect, the light source distal end is displaced by means of an optical detector that senses the presence of a marker on the interface element.

In one aspect, the light source distal end is displaced by means of a texture sensing mechanism.

In one aspect, the interface element is an apertured interface element, the light propagating through each of the apertures without being transmitted through the material from which the interface element is composed. The light may be generated by a CO₂ or Erbium laser.

In one aspect, the apparatus further comprises a scanner for scanning by means of said generated light substantially the entire area of the skin target which underlies the transmitting element at a repetition rate of up to 5 pulses/sec.

In one aspect, the apparatus further comprises a pressure sensor in communication with the interior of the vacuum chamber for determining whether the applied vacuum level is sufficient to inhibit the transmission of pain signals.

In one aspect, the apparatus further comprises a skin contact detector for sensing the placement of the vacuum chamber onto the skin target.

In one aspect, the apparatus is suitable for evacuating air and gel from the vacuum chamber.

In one aspect, the transmitting element is chilled.

In one aspect, the apparatus further comprises means for centering a light source distal end with respect to, and above, walls of the vacuum chamber.

In one aspect, the apparatus further comprises means for repositioning the vacuum chamber to another skin target without gaps or overlaps.

In one aspect, the apparatus further comprises an electronic control unit which is suitable for:
a) receiving a first signal from the skin contact sensor upon placement of the vacuum chamber onto the skin target;
b) transmitting a second signal to a vacuum pump actuator to operate the vacuum pump and to initiate a vacuum applying mode;
c) receiving a third signal from a pressure sensor in communication with the interior of the vacuum chamber when the applied vacuum level is sufficient to inhibit the transmission of pain signals;
d) transmitting a fourth signal to a light source controller to trigger operation of the light source or to enable triggering of the light source;
e) receiving a fifth signal from an optical sensor which is adapted to detect the deactivation of the light source; and
f) transmitting a sixth signal to the vacuum pump actuator to initiate a vacuum release mode.

In one aspect, the apparatus further comprises a dissolving solution pump in fluid communication with a dissolving solution reservoir and with a conduit connected to a vacuum pump discharge, for cleaning and dissolving accumulated gel. Accordingly, the control unit is further adapted to transmit a seventh signal to a dissolving solution pump actuator to activate the dissolving solution pump following a predetermined number of cycles of the vacuum applying and vacuum release mode.

In another embodiment of the invention, the apparatus is suitable for alleviating or preventing pain caused by a non-ablative light-based treatment of a targeted skin structure. Accordingly, the gap separating said the transmitting element from the skin surface adjoining said the skin target and the magnitude of the proximally directed force resulting from said the applied vacuum in combination are suitable for drawing said the skin target to said the vacuum chamber via the opening on the distal end of the vacuum chamber said aperture until said the skin target contacts said the transmitting element; and the control means is suitable for firing the light source after the first predetermined delay, following operation of the vacuum applying means.

In one aspect, the apparatus is suitable for causing the skin target to contact the transmitting element for a duration equal to, or greater than, the first predetermined delay, whereby pain signals generated by the nervous system during the treatment of the skin structure are alleviated or prevented.

The control means is preferably suitable for controlling the -vacuum level generated by the vacuum applying means, and has a plurality of finger depressable buttons, each of which being adapted to set the vacuum applying means and light source at a unique combination of operating conditions so as to generate a predetermined vacuum level within the vacuum chamber and to fire the light source after a predetermined time delay following the operation of the vacuum applying means.

In one aspect, a single light source and vacuum pump are operable in conjunction with differently configured vacuum chambers, for example a vacuum chamber that is suitable for pain alleviation or a vacuum chamber that is suitable for inducing an increase in blood concentration within a skin target. Each differently configured vacuum chamber is releasably attachable to a treatment light handpiece, e.g. by means of suitable threading or clips.

The present invention is also directed to a method for inhibiting pain signals generated by pain receptors in the skin during a skin related medical treatment, comprising the steps of:
a) positioning a rigid interface element above a selected skin region;
b) applying a vacuum of a sufficient value over said skin region such that the latter is flattened and compressed against said interface element; and
c) administering a skin related medical treatment by means adapted to pass through said interface element and to be directed to said compressed skin target, whereby pain signals generated by the nervous system during said medical treatment are inhibited due to the contact of said skin region onto said interface element.

The medical treatment is preferably selected from the group of ultrasonic-based hair removal, ultrasonic-based collagen tightening, ultrasonic-based blood vessel sealing, ultrasonic-based treatment of fatty or cellulite tissue, injections for vaccines, injections for the administration of drugs, injection of collagen, mesotherapy, removal of hair with a hand held implement, needle epilation, injection of collagen within the epidermis, tattoo removal, the treatment of pigmented lesions, and making a small incision in the gums.

In one embodiment, the method further comprises the steps of:
a) placing an evacuation chamber which is provided with an interface element on a skin region in the vicinity of a skin structure;
b) automatically applying a vacuum of a sufficient level to said evacuation chamber following step a) such that said skin region is drawn by the proximally directed force resulting from said vacuum and contacts said interface element;
c) directing a distal end of a light source to said skin region;
d) firing the light source after a predetermined delay following step b) such that the light is directed to said skin structure and effects a desired treatment, whereby pain signals generated by the nervous system during the treatment of said skin structure are alleviated or prevented due to the contact and compression of said skin region onto said interface element for a duration equal to or longer than said predetermined delay;
e) automatically releasing the vacuum from the evacuation chamber following deactivation of the light source;
f) optionally, repositioning the vacuum chamber to the vicinity of another skin region;
g) directing the distal end of the light source to said another skin target; and
h) repeating steps b), d) and e).

In one aspect, the step of directing the distal end of the light source to another skin target is performed by gliding the light source distal end over the interface element.

In one aspect, the step of directing the distal end of the light source to another skin target is performed by means of a scanner.

In one aspect, the delay ranges from approximately 0.5 sec to approximately 4 seconds.

In one aspect, the light source is an intense pulsed monochromatic or non-coherent light source.

In one aspect, the light is in any optical band in the spectral range of 400 to 1800 nm.

The present invention is also directed to apparatus for vacuum-assisted light-based skin treatments. The apparatus comprises a vacuum chamber which is transparent or translucent to intense pulsed monochromatic or non-coherent light directed to a skin target. A vacuum is applied to said vacuum chamber, whereby said skin target is drawn to said vacuum chamber. The efficacy and utility of the apparatus are achieved by employing the apparatus in two modes: (a) in a vacuum applying mode wherein a high vacuum level ranging from 0-1 atmoshpheres is attained and (b) in a vacuum release mode upon deactivation of the light source and of the vacuum pump after optical energy associated with the directed light has been absorbed within a predetermined depth under the skin surface, wherein atmospheric air is introduced to the vacuum chamber so that the vaccum chamber may be speedily repositioned to another skin target.

In one embodiment of the invention, the apparatus comprises:

a) a non-ablative intense pulsed monochromatic or non-coherent light source;

b) a vacuum chamber placeable on a skin target which has an opening on the distal end thereof and provided with a transmitting element on the proximate end thereof, said transmitting element being transparent or translucent to light generated by said source and directed to said skin target;

c) means for applying a vacuum to said vacuum chamber, the level of the applied vacuum suitable for drawing said skin target to said vacuum chamber via said opening; and

d) means for preventing influx of air into vacuum chamber during a vacuum applying mode.

As referred to herein, “distal” is defined as a direction towards the exit of the light source and “proximate” is defined as a direction opposite from a distal direction.

As referred to herein, the term “transmitting element” includes an element through which electromagnetic or ultrasonic energy suitable for effecting a desired treatment is transmitted to a selected skin target. With respect to electromagnetic or ultrasonic energy, the terms “transmitting element” and “interface element” are interchangeable. When the electromagnetic energy is light, the transmitting element is an optical element. When the electromagnetic energy is RF energy, the transmitting element may be metallic.

The terms “evacuation chamber” and “vacuum chamber” as referred to herein are interchangeable.

The vacuum chamber is advantageously one-hand graspable by means of a handle connected thereto so that the vacuum chamber can be held by one hand while a light treatment handpiece is held by the other hand.

Preferably

a) the vacuum applying means comprises a vacuum pump and at least one control valve;

b) the wavelength of the light ranges from 400 to 1800 nm;

c) the pulse duration of the light ranges from 10 nanoseconds to 900 msec;

d) the energy density of the light ranges from approximately 2 to approximately 150 J/cm²;

e) the level of applied vacuum within the vacuum chamber ranges from approximately 0 to approximately 1 atmosphere;

f) the light source is selected from the group of Dye laser, Nd:YAG laser, Diode laser, light emitting diode, Alexandrite laser, Ruby laser, Nd:YAG frequency doubled laser, Nd:Glass laser, a non-coherent intense pulse light source, and a non-coherent intense pulse light source combined with an RF source or with a monopolar or bipolar RF source;

g) the light is suitable for hair removal, collagen contraction, photorejuvenation, treatment of vascular lesions, treatment of sebacouse or sweat glands, treatment of warts, treatment of pigmented lesions, treatment of damaged collagen, skin contraction, treatment of acne, treatment of warts, treatment of keloids, treatment of sweat glands, treatment of psoriasis, and treatment of lesions pigmented with porphyrins or with cyanin green;

h) the light is suitable for the treatment of vascular lesions selected from the group of port wine stains, telangectasia, rosacea, and spider veins;

i) the transmitting element is suitable for transmitting the light in a direction substantially normal to a skin surface adjoining said skin target;

j) the transmitting element is separated from the adjoining skin surface by a gap ranging from 0.5 to 50 mm, and preferably approximately 2 mm;

k) the treatment spot per pulse is greater than 5 mm, and preferably between 15 to 50 mm;

l) the influx of air into vacuum chamber during a vacuum applying mode is prevented by means of a control valve and control circuitry or by means of manual occlusion of a vacuum chamber conduit;

m) the ratio of the maximum length to maximum width of the aperture formed on the distal end of the vacuum chamber ranges from approximately 1 to 4;

n) the vacuum chamber has at least one suction opening, the vacuum being applied to the vacuum chamber via said at least one suction opening;

o) the vacuum chamber is U-shaped; and

p) the vacuum chamber is provided with a rim for sealing the peripheral contact area between the skin surface adjoining the skin target and the vacuum chamber wall.

Preferably, the apparatus further comprises control means for controlling operation of the vacuum pump, the at least one control valve, and the light source. The control means is selected from the group of electronic means, pneumatic means, electrical means, and optical means. The control means may be actuated by means of a finger depressable button, which is positioned on a light treatment handpiece.

In one aspect, the control means is suitable for firing the light source after a first predetermined delay, e.g. from approximately 0.5 sec to approximately 4 seconds, following operation of the vacuum pump.

In one aspect, the control means is suitable for firing the light source after a predetermined delay following opening of the at least one control valve.

In one aspect, the control means is suitable for increasing the pressure in the vacuum chamber to atmospheric pressure following deactivation of the light source, to allow for effortless repositioning of the vacuum chamber to a second skin target. The increase in vacuum chamber pressure may be triggered by means of a light detector which transmits a signal to the control means upon sensing a significant decrease in optical energy generated by the light source or may be effected after a second predetermined delay, following deactivation of the light source.

In one aspect, the control means is suitable for verifying that a desired energy density level of the light is being directed to the skin target and for deactivating the light source if the energy density level is significantly larger than said desired level.

In one aspect, the vacuum chamber is connected to, or integrally formed with, a proximately disposed handpiece through which light propagates towards the skin target. The vacuum chamber has a proximate cover formed with an aperture, said cover being attachable or releasably attachable to a handpiece such as a light guide having an integral transmitting element.

In one aspect, the vacuum pump is an air pump.

In one aspect, the vacuum pump is a pump, e.g. a peristaltic pump, for drawing air and gel from the interior of the vacuum chamber via a hose connected to a conduit in communication with the interior of the vacuum chamber. The hose provides indication means that the skin target has undergone a light-based treatment by means of gel which is discharged from an end of the hose onto a skin surface during a vacuum applying mode.

In one aspect, the apparatus further comprises means to stabilize the vacuum chamber on a substantially non-planar skin surface.

In one aspect, the apparatus further comprises a skin contact detector for sensing the placement of the vacuum chamber onto the skin target and for generating a first signal to activate the vacuum pump following placement of the vacuum chamber onto the skin target.

In one aspect, the control valve is opened following generation of a second signal by means of a light detector which is adapted to sense termination of the light directed to the skin target, atmospheric pressure air thereby being introduced to the interior of the vacuum chamber.

In one aspect, the second signal is suitable for deactivating the vacuum pump.

In another embodiment of the invention, the apparatus further comprises an array of vacuum chambers placeable on a skin surface. The array is formed from a single sheet made of material which is transparent or translucent to the light, said sheet being formed with a plurality of conduits for air evacuation such that each of said conduits is in communication with a corresponding vacuum chamber. The distance between adjacent vacuum chambers is sufficiently small to allow light which has diffused from the interior of each chamber to treat a skin area located underneath a corresponding conduit.

Each conduit preferably branches into first and second portions which are in communication with a vacuum pump and with a source of compressed air, respectively.

In one aspect, each vacuum chamber is provided with a contact detector for triggering a signal to activate the vacuum pump, two control valves to control the passage of fluid through the corresponding first and second conduits portions, respectively, and a light detector which generates a signal to introduce compressed air through the corresponding second conduit portion upon sensing the termination of the light directed to the skin target.

In one aspect, the first conduit portions are arranged such that the air from all vacuum chambers is evacuated simultaneously upon activation of the vacuum pump.

In another embodiment of the invention, the vacuum applying means comprises a vertically displaceable cover to which the transmitting element is secured and chamber walls which surround, and are of a similar shape as, said cover, a vacuum being generated within a vacuum chamber defined by the volume between said cover, said walls, and the skin target upon proximal displacement of said cover relative to said walls. The means for preventing influx into the vacuum chamber is a sealing element which is secured to the outer periphery of the cover and resiliently contacts the chamber walls.

In one aspect, a proximally directed force or distally directed force is generated by any means selected from the group of a plurality of solenoids, a spring assembly, and a pneumatic device, or a combination thereof, which are deployed around the periphery of the cover and connected to the walls, and is controllable so as to adjust the height of the drawn skin target relative to the adjoining skin surface. Due to their low power consumption, a 1.5 V battery may be used to energize the solenoids.

The apparatus preferably further comprises an aeration tube for introducing atmospheric air to the vacuum chamber during a vacuum release mode. The aeration tube is in communication with a valve which is actuated upon conclusion of a skin target treatment.

In one aspect, the proximally directed force is supplemented by means of a vacuum pump.

In another embodiment of the invention, the apparatus comprises means for preventing passage of skin cooling gel to the vacuum applying means.

In one aspect, the means for preventing passage of gel to the vacuum applying means comprises a trap, a first conduit through which gel and air are drawn from the vacuum chamber to said trap, a second conduit through which air is drawn from said trap to the vacuum pump, and optionally, a filter at the inlet of the first and second conduits.

In one aspect, the trap is suitable for the introduction therein of an ion exchange resin with which the gel is boundable.

In one aspect, the means for preventing passage of gel is a detachable vacuum chamber upper portion, detachment of said upper portion allowing removal of gel retained within the vacuum chamber interior. Suitable apparatus comprises an upper portion having an open central area, a transmitting element attached to said upper portion, vacuum chamber walls, a vacuum chamber cover perpendicular to said walls and suitably sized so as to support said upper portion, and a plurality of attachment clips pivotally connected to a corresponding vacuum chamber wall for detachably securing said upper portion to said vacuum chamber cover.

In one aspect, the vacuum chamber walls are coated with a hydrophobic material. Accordingly, the vacuum chamber provides indication that the skin target has undergone a light-based treatment by means of gel which falls to the skin surface during a vacuum release mode in the shape of the distal end of the vacuum chamber walls.

In one aspect, the at least one suction opening is sufficiently spaced above the distal end of a vacuum chamber wall and from the centerline of the vacuum chamber so as to prevent obstruction of the at least one suction opening by gel and drawn skin upon application of the vacuum.

In another embodiment of the invention, the apparatus further comprises means for skin cooling, said skin cooling means adapted to reduce the rate of temperature increase of the epidermis at the skin target. The level of the applied vacuum is suitable for evacuating condensed vapors which are produced within the gap between the transmitting element and the skin target and condense on the transmitting element during the cooling of skin.

In one aspect, the skin cooling means is a metallic plate in abutment with the vacuum chamber on the external side thereof, said plate being cooled by means of a thermoelectric cooler. The plate may be positionable on the skin surface adjoining said skin target in order to cool the lateral sides of the vacuum chamber or may be in contact with the transmitting element.

In one aspect, the skin cooling means is a polycarbonate layer transparent to the directed light which is attached to the distal face of the transmitting element.

In one aspect, the skin cooling means is a gel, a low temperature liquid or gas applied onto the skin target.

In another embodiment of the invention, the apparatus is suitable for controlling the depth of light absorption by blood vessels under a skin surface, comprising:

a) a vacuum chamber placed on a skin target which is formed with an aperture on the distal end thereof and provided with a transmitting element on the proximate end thereof, said transmitting element being transparent or translucent to intense pulsed monochromatic or non-coherent light directed to said skin target and suitable for transmitting the light in a direction substantially normal to a skin surface adjoining said skin target;

b) means for applying a vacuum to said vacuum chamber, the level of the applied vacuum suitable for drawing said skin target to said vacuum chamber via said aperture; and

c) means for inducing an increase in the concentration of blood and/or blood vessels within a predetermined depth below the skin surface of said skin target, optical energy associated with the directed light being absorbed within said predetermined depth.

As referred to herein, the term “blood volume fraction” is interchangeable with “the concentration of blood and/or blood vessels within a predetermined depth below the skin surface”.

In one embodiment, the means for inducing an increase in the concentration of blood and/or blood vessels within a predetermined depth below the skin surface of said skin target is a means for modulating the applied vacuum.

The depth under the skin surface at which optical energy is absorbed may be selected in order to thermally injure or treat predetermined skin structures located at said depth. As referred to herein, a “skin structure” is defined as any any damaged or healthy functional volume of material located under the epidermis, such as blood vessels, collagen bundles, hair shafts, hair follicles, sebacious glands, sweat glands, adipose tissue. Depending on the blood concentration within the skin target, the light may propagate through the skin surface and upper skin layers without being absorbed thereat and then being absorbed at a skin layer corresponding to that of a predetermined skin structure. As referred to herein, the term “light” means both monochromatic and non-coherent light. The terms “light absorption” and “optical energy absorption” refer to the same physical process and are therefore interchangeable.

In contrast with a prior art vacuum-assisted apparatus for laser or intense pulsed light treatment wherein a sharp skin fold is produced through a slit following application of the vacuum, vacuum-assisted drawn skin by means of the apparatus of the present invention is not distorted, but rather is slightly and substantially uniformly drawn to the vacuum chamber, protruding approximately 1-2 mm from the adjoining skin surface. The maximum protrusion of the drawn skin from the adjoining skin surface is limited by a transmitting element defining the proximate end of the vacuum chamber. The transmitting element is separated from the adjoining skin surface by a gap of preferably 2 mm, and ranging from 0.5-50 mm. In one embodiment of the invention, the drawn skin abuts the transmitting element.

As referred to herein, “vacuum modulation” means adjustment of the vacuum level within, or of the frequency by which vacuum is applied to, the vacuum chamber. By properly modulating the vacuum, the blood flow rate, in a direction towards the vacuum chamber, within blood vessels at a predetermined depth below the skin surface can be controlled. As the concentration of blood and/or blood vessels is increased within the skin target, the number of light absorbing chromophores is correspondingly increased at the predetermined depth. The value of optical energy absorbence at the predetermined depth, which directly influences the efficacy of the treatment for skin disorders, is therefore increased.

Preferably

a) The wavelength of the light ranges from 400 to 1800 nm.

b) The pulse duration of the light ranges from 10 nanoseconds to 900 msec.

c) The energy density of the light ranges from 2 to 150 J/cm².

d) The ratio of the maximum length to maximum width of the aperture formed on the distal end of the vacuum chamber ranges from approximately 1 to 4.

e) The level of the applied vacuum within the vacuum chamber ranges from 0 to 1 atmosphere.

f) The frequency of vacuum modulation ranges from 0.2 to 100 Hz.

g) The light is fired after a predetermined delay following application of the vacuum.

h) The predetermined delay ranges from approximately 10 msec to approximately 1 second.

i) The duration of vacuum application to the vacuum chamber is less than 2 seconds.

j) Vacuum modulation is electronically controlled.

In one embodiment of the invention, the means for inducing an increase in the concentration of blood and/or blood vessels within a predetermined depth below the skin surface of said skin target is at least one support element positioned at a skin area adjoining the skin target and having a thickness suitable for inducing an increase in the concentration of blood and/or blood vessels within said predetermined depth. The apparatus may further comprise at least one leg having a thickness considerably less than the at least one support element and positioned at the periphery of the vacuum chamber, said at least one leg being separated from an adjacent support element, the at least one support element being adapted to urge blood expelled by said at least one leg towards the skin target.

The predetermined depth under the skin surface at which optical energy is absorbed is selected in order to thermally injure or treat predetermined skin structures located at said depth.

Due to implementation of the apparatus, the treatment energy density level for various types of treatment is significantly reduced, on the average of 50% with respect with that associated with prior art devices. The treatment energy density level is defined herein as the minimum energy density level which creates a desired change in the skin structure, such as coagulation of a blood vessel, denaturation of a collagen bundle, destruction of cells in a gland, destruction of cells in a hair follicle, destruction of unwanted lesions by means of photodynamic therapy, or any other desired effects. The following is the treatment energy density level for various types of treatment performed with use of the present invention:

a) treatment of vascular lesions, port wine stains, telangectasia, rosacea, and spider veins with light emitted from a dye laser unit and having a wavelength of 585 nm: 5-12 J/cm²;

b) treatment of vascular lesions, port wine stains, telangectasia, rosacea, and spider veins with light emitted from a diode laser unit and having a wavelength of 940 nm: 10-30 J/cm²;

c) treatment of vascular lesions with light emitted from an intense pulsed non-coherent light unit and having a wavelength of 570-900 nm: 5-20 J/cm²;

d) photorejuvination with light emitted from a dye laser unit and having a wavelength of 585 nm: 1-4 J/cm²;

e) photorejuvination with light emitted from an intense pulsed non-coherent light unit and having a wavelength of 570-900 nm: 5-20 J/cm²;

f) photorejuvination with a combined effect of light emitted from an intense pulsed non-coherent light unit and having a wavelength of 570-900 nm and of a RF source: 10 J/cm² for both the intense pulsed non-coherent light unit and RF source;

g) hair removal with light emitted from a Nd:YAG laser unit and having a wavelength of 1604 nm: 25-35 J/cm²; and

h) Porphyrin-based photodynamic therapy with light emitting diodes delivering blue light (420 nm), orange light (585 nm), or red light (630 nm): 5-20 J/cm².

The preferably further comprises a control unit for controlling operation of the vacuum applying means and light source. The control unit is also suitable for controlling operation of at least one control valve in communication with the vacuum chamber, for firing the light after a predetermined delay following application of the vacuum, and for electronically modulating the vacuum.

In one aspect, the apparatus further comprises a skin contact detector for sensing the placement of the vacuum chamber onto the skin target, the control unit being suitable for activating the vacuum applying means in response to a signal transmitted by said skin contact detector.

In one aspect, the apparatus further comprises a light detector for sensing the termination of the light directed to the skin target, the control unit being suitable for regulating a control valve in response to a signal transmitted by said light detector so as to introduce atmospheric pressure air to the interior of the vacuum chamber.

In one aspect, the apparatus further comprises a pulsed radio frequency (RF) source for directing suitable electromagnetic waves to the skin target. The frequency of the electromagnetic waves ranges from 0.2-10 MHz. The RF source is either a bipolar RF generator which generates alternating voltage applied to the skin surface via wires and electrodes or a monopolar RF generator with a separate ground electrode. The control unit is suitable for transmitting a first command pulse to the at least one control valve and a second command pulse to both the intense pulsed light source and RF source.

In one aspect, the apparatus-further comprises an erythema sensor, said sensor suitable for measuring the degree of skin redness induced by the vacuum applying means. The control unit is suitable for controlling, prior to firing the light source, the energy density of the light emitted from the light source, in response to the output of the erythema sensor.

In one aspect, the vacuum chamber has a proximate cover formed with an aperture, said cover being attachable to a handpiece, such as a light guide, having an integral transmitting element.

In one aspect, the apparatus further comprises means for skin cooling, said skin cooling means adapted to reduce the rate of temperature increase of the epidermis at the skin target.

In one aspect, the apparatus further comprises means for preventing passage of skin cooling gel to the vacuum applying means.

### BRIEF DESCRIPTION OF THE DRAWINGS

In the drawings:

FIG. 1 schematically illustrates a portion of the nervous system that is involved in the generation of a pain inhibiting signal as a skin region is flattened following the application of a suitable vacuum level thereto;

FIG. 2 schematically illustrates apparatus that is suitable for inhibiting pain during an ultrasound-based medical treatment;

FIG. 3 schematically illustrates an evacuation chamber and the corresponding interface element that are suitable for inhibiting pain during an injection into a target skin region, according to one embodiment of the invention;

FIG. 4 schematically illustrates the tensile forces to which an interface element is normally subjected as a vacuum is applied to an evacuation chamber;

FIG. 5 schematically illustrates the factory-induced compressive forces that counteract the tensile forces of FIG. 4;

FIGS. 6a-e schematically the generation of factory-induced compressive forces by press fitting an interface element in a retaining member;

FIG. 7a schematically illustrates an interface element that is subjected to compressive forces;

FIG. 7b schematically illustrates the aspect ratio of the interface element of FIG. 7a after being subjected to compressive forces;

FIG. 7c schematically illustrates a subdivided interface element by which the aspect ratio may be reduced;

FIG. 8 schematically illustrates the administering of an injection by a small injection angle to thereby increase the tear resistance of the interface element;

FIG. 9 schematically illustrates an injection method by which a needle pierces through the puncturable sidewalls of an evacuation chamber;

FIGS. 10a-b schematically illustrate a convex and concave interface element, respectively;

FIG. 11 illustrates a bar chart of the pain level distribution of patients that underwent light-based skin treatments, comparing the pain sensation of a vacuum-assisted treatment with a treatment that was not vacuum-assisted;

FIG. 12 schematically illustrates an evacuation chamber having a concave interface element in which a vacuum is generated by means of a pre-evacuated vacuum source;

FIG. 13 schematically illustrates an evacuation chamber having an apertured interface element;

FIGS. 14a-b illustrate an apertured interface element having a shield element, wherein FIG. 14a illustrates the shield element as it is covering the interface element and FIG. 14b illustrates the interface element after the shield element has been removed therefrom;

FIG. 15 schematically illustrates a vibrating interface element;

FIG. 16 schematically illustrates an apparatus for automatically administering a plurality of injection needles;

FIG. 17 schematically illustrates means for maintaining the vacuum within an evacuation chamber following deactivation of the vacuum pump;

FIG. 18 schematically illustrates an evacuation chamber configured as a slit;

FIG. 19 schematically illustrates a painless tweezers-based hair removal procedure carried out in conjunction with the evacuation chamber of the present invention;

FIGS. 20a-b schematically illustrate an apparatus according to one embodiment of the invention which does not require a vacuum pump for administering repeated painless injections by a single medical professional wherein FIG. 20a illustrates the positioning of the evacuation chamber before the application of vacuum therein and FIG. 20b illustrates the administration of an injection after a skin region is drawn by the vacuum applied to the evacuation chamber;

FIG. 21 schematically illustrates an apparatus for painlessly administering a dual light and needle based medical treatment;

FIG. 22 is a schematic drawing of apparatus in accordance with another embodiment of the invention, which is suitable for alleviating pain during a light-based skin treatment;

FIG. 23 schematically illustrates a evacuation chamber which is configured to induce the expulsion of blood from a skin target to a peripheral skin area;

FIG. 24 schematically illustrates a treatment handpiece held by one hand which comprises a light source and a evacuation chamber;

FIG. 25 is a schematic perspective view of a sapphire transmitting element that is suitable for transmitting both light and RF waves to a skin target.

FIG. 26 schematically illustrates a large sized evacuation chamber used for pain alleviation in conjunction with a monopolar RF source;

FIG. 27 schematically illustrates a large sized evacuation chamber used for pain alleviation in conjunction with a bipolar RF source;

FIG. 28 schematically illustrates a evacuation chamber provided with a pressure sensor;

FIG. 29a schematically illustrates a side view of an array of diverging lenses, for an improved rate of healing for tissue that has been treated by laser treatment light;

FIG. 29b schematically illustrates in plan view the energy distribution of the treatment light transmitted through the array of lenses of FIG. 29a onto the underlying skin surface;

FIG. 30 is a schematic drawing of an exemplary skin cooling device, which is suitable for the apparatus of FIG. 22;

FIG. 31 schematically illustrates a skin chiller that emits a skin chilling spray;

FIGS. 32A and 32B schematically illustrate the accumulation of gel as a evacuation chamber is displaced from skin area to another;

FIG. 33 is a schematic drawing of an exemplary trap, for preventing the passage of gel to a vacuum pump;

FIG. 34 is a schematic perspective drawing of apparatus in accordance with another embodiment of the invention, illustrating a detachable upper portion of a evacuation chamber;

FIG. 35 is a photograph of the back of a patient, illustrating the efficacy of the hair removal treatment of the invention;

FIG. 36 schematically illustrates a evacuation chamber to which a vacuum is applied by means of a peristaltic pump;

FIGS. 37A-C illustrate the production of a evacuation chamber by a vertically displaceable cover in three stages;

FIG. 38 schematically illustrates another embodiment of the invention wherein gliding apparatus is used to displace a laser or IPL distal end along a large sized transmitting element of a pain inhibiting evacuation chamber;

FIGS. 39a and 39b illustrate top and side views, respectively, of a evacuation chamber transmitting element which is provided with another configuration of bipolar RF-assisted metallic conducting electrodes that facilitate a gliding apparatus;

FIGS. 40a and 40b schematically illustrate two embodiments of a gliding apparatus, respectively;

FIG. 41 is a schematic drawing which illustrates the propagation of an intense pulsed laser beam from a handpiece to a skin target according to a prior art method;

FIG. 42 is a schematic drawing which illustrates the propagation of an intense pulsed non-coherent light beam from a handpiece to a skin target according to a prior art method;

FIG. 43 is a schematic drawing of a prior art treatment method by which pressure is applied to a skin target, in order to expel blood from those portions of blood vessels which are in the optical path of subcutaneously scattered light;

FIG. 44 is a schematic drawing of a prior art vacuum-assisted rolling cellulite massage device;

FIG. 45 is a schematic drawing of a prior art vacuum-assisted hair removal device adapted to reduce the blood concentration within a skin fold formed thereby, in order to illuminate two opposed sides of the skin fold and consequently remove melanin-rich hair shafts;

FIG. 46 schematically illustrates a evacuation chamber which is configured to induce blood transfer from a peripheral skin area to a skin target;

FIG. 47 is a schematic drawing of apparatus in accordance with one embodiment of the present invention, employing a manually occluded U-shaped evacuation chamber;

FIG. 48 is a schematic drawing of apparatus in accordance with another embodiment of the present invention, employing an electronically controlled evacuation chamber;

FIG. 49 is a schematic drawing of apparatus in accordance with the present invention, employing an intense pulsed non-coherent light source;

FIGS. 50a and 50b schematically illustrate a evacuation chamber which is attachable to a light guide, wherein FIG. 50a illustrates the evacuation chamber prior to attachment and FIG. 50b illustrates the evacuation chamber following attachment;

FIG. 51 is a schematic drawing of apparatus in accordance with the present invention, which is provided with a skin chiller;

FIG. 52 is a drawing which schematically illustrates the effect of applying a subatmospheric pressure to a evacuation chamber in order to increase the blood concentration in skin drawn towards the evacuation chamber;

FIG. 53 is a drawing which schematically illustrates the increased concentration of a plurality of blood vessels in a skin target following application of a vacuum to a evacuation chamber, resulting in increased redness of skin and enhanced absorption of light;

FIG. 54 is an enhanced photograph illustrating the change in skin color to a pinker color following the application of a vacuum in accordance with the present invention prior to treatment of a fine wrinkle;

FIG. 55 is a schematic drawing of another embodiment of the invention, illustrating propagation of intense pulsed light from an external light source to a transparent modulated evacuation chamber;

FIG. 56 schematically illustrates another embodiment of the invention which employs both an intense pulsed light source and a radio frequency source, for improved coagulation of blood vessels;

FIG. 57 schematically illustrates a pivotable scanner that is used in conjunction with a large sized pain inhibiting evacuation chamber;

FIG. 58 is a flow chart of a method for synchronizing the operation of a laser beam scanner with respect to that of a pain inhibiting vacuum pump;

FIG. 59 schematically illustrates a kaleidoscopic square beam homogenizer which enables the homogeneous scanning of a laser beam without overlap on a evacuation chamber transmitting element;

FIG. 60 schematically illustrates means for centering a light source distal end with respect to a evacuation chamber;

FIG. 61 is a schematic drawing of apparatus in accordance with yet another embodiment of the invention; and

FIG. 62A is a plan view of an array of evacuation chambers and FIG. 62B is a cross sectional view thereof, taken about plane A-A of FIG. 62A.

### DESCRIPTION OF PREFERRED EMBODIMENTS

The present invention relates to apparatus adapted to alleviate or prevent the normal pain which is sensed during a skin-related medical treatment, such as an ultrasonic treatment of the skin or during an injection into the skin. The apparatus of the present invention includes an evacuation chamber having a rigid surface overlying an aperture formed in a skin engaging lower portion. When the evacuation chamber is placed on a selected skin region and a vacuum of a sufficiently high level is applied to the skin region, the skin region is drawn through the aperture towards, and is compressed against, the rigid surface. The rigid surface may extend from the upper edge of the evacuation chamber walls or may be retained within a cover element connected to, or integrally formed with, the evacuation chamber walls. Due to the compression of the skin against the rigid surface, pain signals generated by pain receptors located within the skin region during a medical treatment of the skin are alleviated or prevented.

Medical treatments that have been heretofore painful to patients may be administered in a painless fashion with the use of the apparatus of the present invention. Typical medical treatments that may be administered in painless fashion are ultrasonic-based hair removal, ultrasonic-based collagen tightening, ultrasonic-based blood vessel sealing, ultrasonic-based treatment of fatty or cellulite tissue, injections for vaccines, injections for the administration of drugs, injection of collagen, mesotherapy, removal of hair with a hand held implement, and needle epilation.

Needle epilation, which is carried out by inserting a needle into the skin along a hair shaft to a depth of a few millimeters, may be performed with use of the apparatus of the present invention without causing pain to the patient. The duration of this procedure is approximately one second, including the time needed to insert the needle and to destroy a single hair follicle. While the procedure is very effective in terms of heating the follicle by means of the needle and destroying the critical organs necessary for hair regeneration and growth by applying an electrical current, it is very painful. By applying a vacuum and inducing compression of the drawn skin region against a rigid surface of the evacuation chamber, the pain sensation is inhibited while efficacious hair destruction is maintained. The apparatus of the present invention will also facilitate painless hair removal at skin regions, such as the face, particularly for women suffering from an excess in androgenic hormones, for which laser or IPL light, i.e. photepilation, is not suitable. Another advantage of the apparatus of the present invention is that thin and white hairs, which cannot be removed by photoepilation, will be able to be removed by needle epilation in painless fashion.

To determine operability of the apparatus of the present invention, the degree of vacuum-assisted pain alleviation was evaluated according to a modified McGill pain questionnaire. The McGill pain questionnaire is well known to pain specialists, and is described by R. Melzack, “The McGill Pain Questionnaire: Major Properties and Scoring Methods,” Pain 1 (1975), pp. 277-299. The sensed pain associated with 45 skin targets following a light-based treatment, i.e. by means of laser or IPL light, of vacuum-induced flattened skin was compared to the pain associated with light-based treatments conducted without skin flattening. A dramatic pain reduction, from an average of pain level 4, which is indicative of a very painful treatment, to an average of pain level 2, which is indicative of a lack of pain, was revealed.

The inventors have found that an applied vacuum level of at least 150-200 mmHg, and preferably at least 400 mmHg, is generally needed to alleviate pain. A lower vacuum level, such as of 50-100 mmHg, has been found to be not sufficient for the alleviation of pain.

It will be appreciated that the Gate Theory of Afferent Inhibition, which states that a pressure signal sensed by large, fast-conducting tactile nerves excludes access for the weaker pain signal and therefore inhibits the pain signal transmission by pain nerves in the spinal cord, has been established only during the application of an external positive pressure to the skin region. The Gate Theory of Afferent Inhibition has never been evaluated heretofore with respect to the application of a negative pressure to the skin region, whereby internal upward pressure-derived forces are generated within the skin and may affect the pain signal transmission in different ways.

It will be appreciated that the application of a suitable vacuum over a skin region which causes the latter to be flattened by an overlying rigid surface is physiologically not equivalent to the application of positive pressure over the skin.

Applying a positive pressure onto a skin surface compresses and squeezes the same. Bones located under the skin surface apply a reactive force and therefore increase the degree of skin compression, as well as to the squeezing of blood vessels and of nerves bundles. The physiological reaction to the pressing of skin depends on the skin thickness, and particularly, on the distance of the bones from the skinsurface.

In contrast, bones underlying a skin surface drawn by a vacuum applied thereto are not influential during a skin flattening procedure. Since the underlying bones do not apply a reactive force as the connective tissue overlying these bones is drawn towards the evacuation chamber, the degree to which blood vessels and nerve bundles within a drawn skin region are compressed is reduced. Thus the physiological processes of connective tissue associated with a vacuum induced skin flattening procedure are different than those of connective tissue which is compressed as a result of the application of positive pressure thereto. The inventors are not aware of any published clinical studies which describe the effects of a vacuum induced skin flattening procedure. Any clinical results of a study regarding the application of positive pressure over a skin surface are not expected to be clinically relevant to those obtainable with respect to a vacuum induced skin flattening procedure.

The generation of a vacuum to a skin region may be advantageously controlled, in order to ensure that the skin region will be flattened prior to the medical treatment and to achieve a predetermined rate of repeatability.

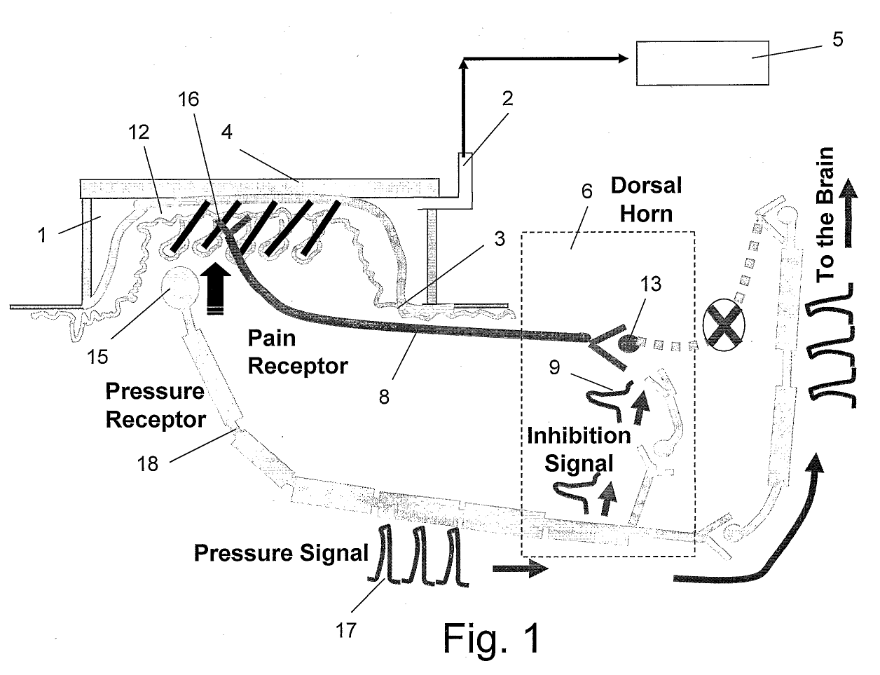

The inventors have surprisingly found that a contributory factor to the level of vacuum-assisted pain reduction is the surface area of the rigid surface. Without wishing to be limited by any particular theory, the inventors believe that the relationship between the level of vacuum-assisted pain reduction and the surface area of the rigid surface is reflected in FIG. 1. As schematically illustrated, evacuation chamber 1 is shown to be placed above skin region 12, which has been selected as the target of a normally painful skin-related medical treatment. The air in evacuation chamber 1 is evacuated by means of a vacuum pump 5 via conduit 2 in communication with evacuation chamber 1. Following application of the vacuum within evacuation chamber 1, skin region 12 is upwardly drawn within the interior of evacuation chamber 1, contacts planar rigid surface 4, and is flattened thereby. A rigid surface 4 of evacuation chamber 1 having a sufficiently large area ensures that a correspondingly large number of pressure receptors 15 will be compressed. Pressure receptors 15 sense the compression of skin region 12 as it is flattened by rigid surface 4, and fast conducting myelinated pressure nerve 18 located within skin region 12 transmits a generated pressure signal 17 to dorsal horn 6 of the spinal cord.

Pressure signal 17 functions as an inhibition signal 9 within dorsal horn 6 at a synaptic connection 13 to the brain, thereby inhibiting the pain signal, which is normally transmitted to the brain via the spinal cord through slower non-myelinated pain nerve 8 after being sensed by pain receptor 16 as a result of a pain generating medical treatment, from being transmitted to the brain. The inhibition of the transmission of pain signal impulses to the brain by pressure signals 17 is of chemical origin. Once inhibition signal 9 reaches synaptic connection 13, a negative charge is generated which inhibits the activation of the pain nerve which is in communication with the brain.

If the area of rigid surface 4 is not sufficiently large, fewer pressure receptors 15 will be compressed and a pain sensation will be felt due to the transmission of the corresponding pain signal to the brain from pain nerves which are not gated by pressure receptors. Pain reduction has been found to be noticeable in skin regions such as the back, hands, legs and armpits when the rigid surface has an area greater than at least 100 mm², and preferably greater than 200 mm², such as one that has a length of 20 mm and a width of 40 mm.

The inventors have also surprisingly found that the pain signals cease to be inhibited when the duration of the applied vacuum is longer than a predetermined value. When the duration of the applied vacuum is longer than approximately 6 seconds, depending on the vacuum level and the surface area of the rigid surface against which the skin region is compressed, the compression of the drawn skin against the rigid surface does not provide a pain inhibiting effect. A medical treatment administered to skin region 12 is liable to very painful if a pain inhibition signal is not generated during the treatment, e.g. when the evacuation chamber, if one exists, is not suitable for drawing skin in compressing fashion against the rigid surface, or the vacuum applying duration is longer than approximately 6 seconds and the delay between the generation of the vacuum and the medical treatment administered to the skin target is significantly greater than 6 seconds. The maximum vacuum applying duration that provides a pain inhibiting effect may change depending on the individual patient and the location of the bodily part to which the medical treatment is administered.

The inventors have also surprisingly found that the pain signals continue to be inhibited for approximately 2-3 seconds after release of the vacuum. Since the pain inhibition effect continues after release of the vacuum, a painless injection may be administered immediately after the vacuum is released and the evacuation chamber or a portion thereof is removed from the target skin region. Such a procedure is advantageous for those skin compression conditions which prevent a sufficient amount of injection material to be introduced into the skin. Despite the relative large volume of beneficial material that is introduced into a skin region during an injection, eg. 1 cm³, the inventors have surprisingly found that the presence of such beneficial material within the epidermis and dermis as it is being absorbed within the body does not cause any discomfiture as a result of the pain inhibition. The inventors have also surprisingly found that the beneficial material that is intradermally injected is not rejected from the body despite the increased volume of the skin region as the vacuum is applied.

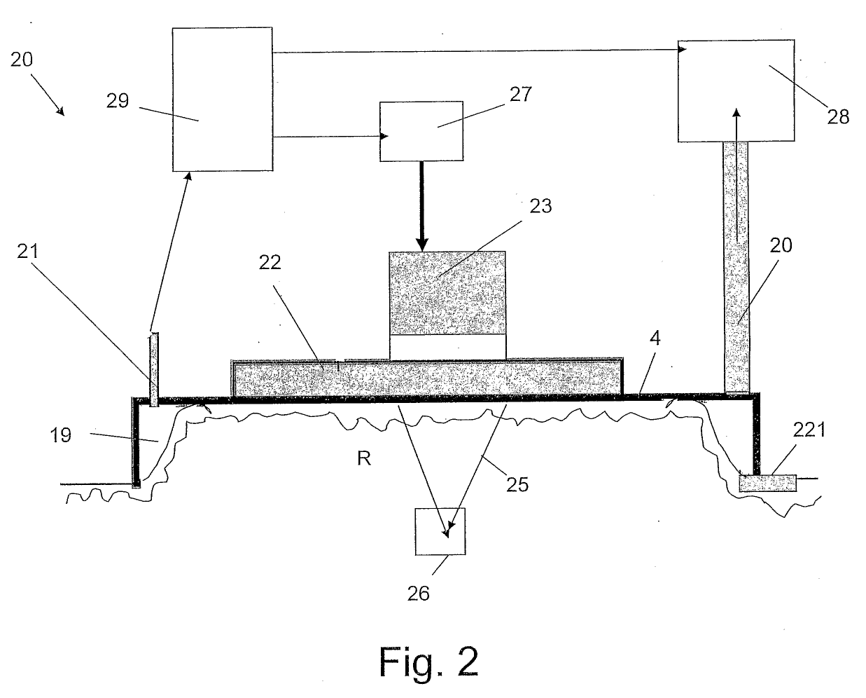

FIG. 2 schematically illustrates an embodiment of the present invention which is suitable for alleviating pain caused by a skin-related ultrasonic treatment. The apparatus, which is generally indicated by numeral 20, comprises evacuation chamber 19 placed on a skin region R, planar rigid surface 4 of evacuation chamber 19 overlying skin region R, interface element 22 placed on, or connected to, rigid surface 4 through which ultrasonic waves propagate before impinging upon the drawn skin region R, vacuum pump 28, and electronic control unit 29. Evacuation chamber 19 may have a height of 7 mm and an area of 20×40 mm, although an evacuation chamber area of 20×50 mm, 12×12 mm, 12×50 mm, 20×50 mm, or any other suitable pain inhibiting dimension is acceptable. Interface element 22, which is made of a material which is transparent to ultrasonic waves, e.g. polyethylene having a thickness of 2 mm, thick rubber, or thick silicon, is sufficiently rigid to prevent surface 4 from flexing as skin region R is compressed thereagainst, thereby increasing the degree of skin compression and the corresponding degree of pain inhibition.

The ultrasonic waves having a frequency ranging from 1-10 MHz are generated by means of ultrasonic transducer 23, e.g. made from PZT and dimensioned with a length of 20 mm. Ultrasonic transducer 23, which is suitable for treating subcutaneous target 26, such as malignant and non-malignant lesions, fatty tissue, cellulite tissue, a blood vessel or a hair follicle, can be configured as a concave reflector such as produced by Ultrashape Ltd., Israel, which is suitable for emitting a focused beam 25 impinging upon target 26. Alternatively, focusing may be achieved by a phased array technique. Transducer 23 is activated by electronic power supply 27, e.g. produced by General Electric.

Vacuum pump 28, which is adapted to evacuate the interior of evacuation chamber 19 via conduit 20, is activated by means of skin contact sensor 221, e.g. an opto-coupler or a microswitch well known to those skilled in the art. Skin contact sensor 221 is in electrical communication with electronic control unit 29 and is adapted to detect the placement of evacuation chamber 19 in the vicinity of skin region R. Vacuum pump 28 may be driven by an inexpensive DC motor or an AC motor. Alternatively, vacuum pump 28 may be a dual air-gel vacuum pump described in copending U.S. patent application Ser. Nos. 11/057,542 and 11/401,674 by the same applicant, the description of which is incorporated herein by reference, when skin region R is coated by gel.

A pressure sensor 21 in communication with the interior of evacuation chamber 19 is capable of detecting the generated vacuum level therewithin, to ensure that vacuum pump 28 will automatically generate a predetermined pain inhibiting vacuum level ranging between 400 mmHg and 1 atmosphere. Pressure sensor 21 transmits a signal indicative of the generated vacuum level to electronic control unit 29, which controls both vacuum pump 28 and power supply 27 of ultrasonic transducer 23.

Electronic unit 29 therefore controls apparatus 20 according to the following sequence. After skin contact sensor 221 detects the placement of evacuation chamber 19 on a skin surface in the vicinity of skin region R, vacuum pump 28 is activated in order to apply a suitable pain inhibiting vacuum level within evacuation chamber 19, as detected by pressure sensor 21. Following generation of the pain inhibiting vacuum level, which is generally achieved within less than 0.5 second, power supply 27 of ultrasonic transducer 23 is activated by power supply 27 to commence the medical treatment directed at target 26. Power supply 27 is then deactivated in order to terminate the medical treatment after a predetermined duration, e.g. 1.5 sec, corresponding to a treatment duration of 1 sec and a delay thereafter of 0.5 sec. Vacuum pump 28, or an additional pump which is not shown, is then commanded to release the vacuum within evacuation chamber 19.

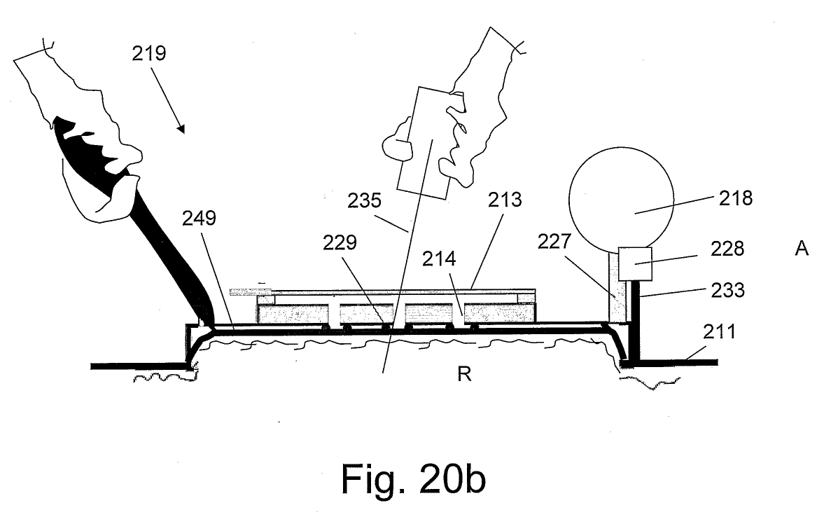

In the embodiment of FIG. 19, painless hair removal is carried out by removing a bundle of hair shafts with the use of tweezers. To enable the introduction of tweezers 184 into evacuation chamber 12, outer and inner interface elements are employed. Outer interface element 185 is planar and horizontally extending and is formed with a central aperture (not shown), in which inner interface element 186 is inserted, such as with a press fit. Inner interface element 186 is a vertically extending cylinder, and its inner diameter is substantially equal to the outer diameter of piston 181 to which is connected tweezers 184. When the vacuum is being applied, inner interface element 186 is covered and skin region R is compressed against the bottom surface of outer interface element 185 and inner interface element 186. The inner interface element cover (not shown) is then removed and piston 181 is introduced into interior 188 of inner interface element 186 as shown so that tweezers 184 will be able to grasp and pull hair shafts 189 through the inner interface element interior 188. Since piston 181 substantially occludes interior 188, the vacuum level within evacuation chamber 182 is not significantly reduced and hair shafts 189 may therefore be painlessly removed. This hair removal procedure is very painful when a vacuum is not generated within evacuation chamber 182 and skin region R is not compressed against outer interface element 185.

Pain Inhibition During Injections

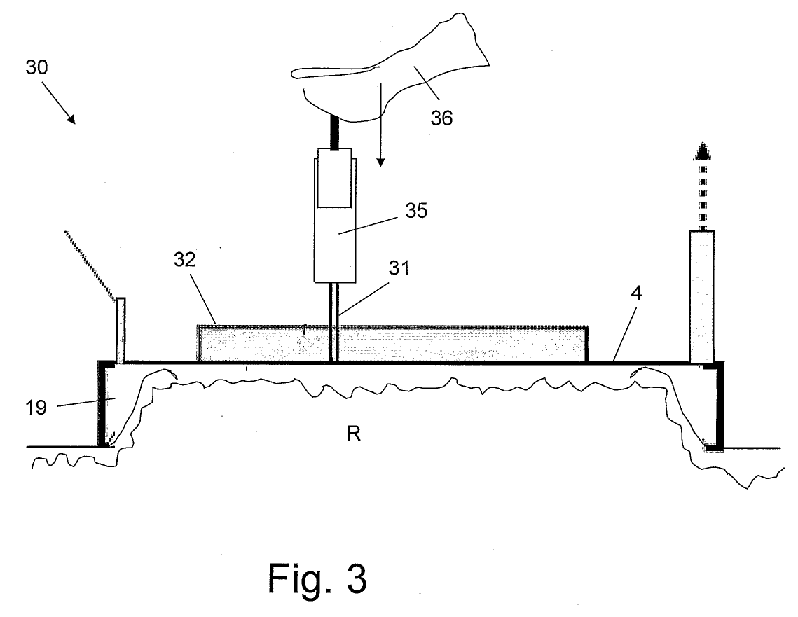

FIG. 3 schematically illustrates an embodiment of the present invention which is suitable for ensuring painless injections. With the exception of interface element 32, apparatus 30 is similar to apparatus 20 of FIG. 2. As a vacuum is applied to evacuation chamber 19, skin region R is flattened by rigid surface 4. The application of the vacuum allows skin region R to be drawn towards, and compressed by, rigid surface 4, thereby alleviating the immediate sharp pain which is normally sensed during the injection of needle 31 and enabling a larger skin volume to accept the injected material. Rigid surface 4 and interface element 32 are made of a puncturable material, so that when needle applicator 35 is depressed by the finger 36 of a health professional, needle 31 is downwardly displaced, penetrating interface element 32, rigid surface 4, and skin region R. Interface element 32 is preferably dimensioned to optimize the level of pain inhibition.

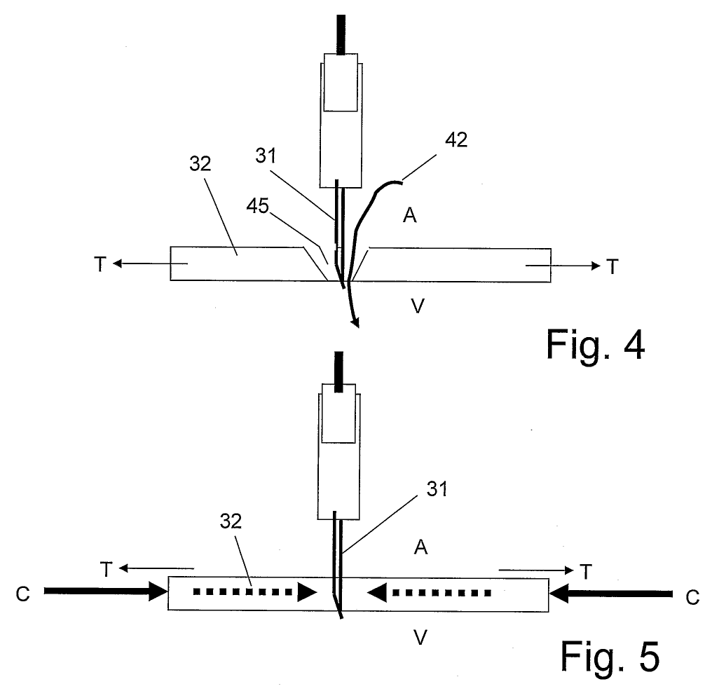

As shown in FIG. 4, interface element 32 is normally subjected to tensile forces T after being punctured by needle 31 if any of the measures which will be described hereinafter are not taken. Due to the pressure differential between ambient pressure side A and vacuum side V of interface element 32, stream of air 42 flows through puncture site 45, acting on the surrounding walls of the puncture site and causing the puncture site to be enlarged as a result of the influence of tensile forces T which pull interface element 32 to the periphery of the evacuation chamber. Over the course of time, interface element 32 is liable to tear and the vacuum level within the evacuation chamber is liable to be drastically reduced due to the passage of air through puncture site 45.

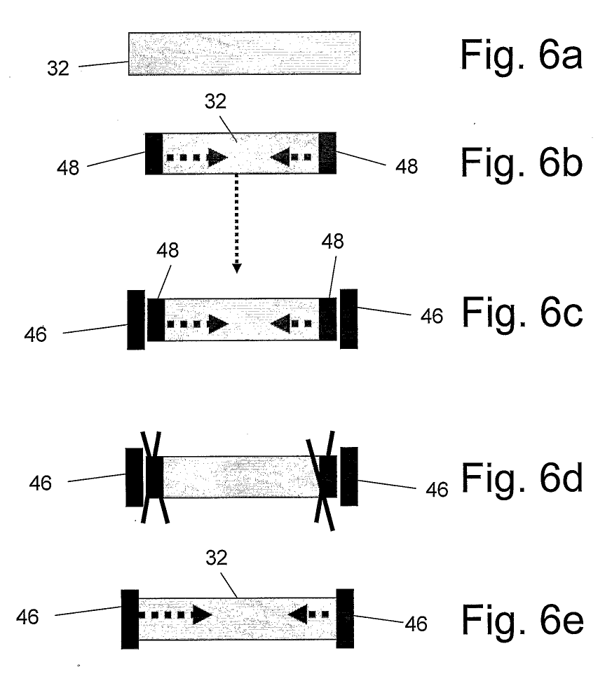

To counteract the effect of the tensile forces, interface element 32 may be factory-produced under compression. As shown in FIG. 5, compressive forces C directed towards the center of interface element 32 counteract the tensile forces T that are produced following the penetration of needle 31 into the interface element and the subsequent passage of air through the puncture site due to the pressure differential between ambient pressure side A and vacuum side V of interface element 32. Exemplary suitable puncturable materials that can be easily subjected to factory-produced compressive forces are cork and thin polymeric materials, e.g. silicon having a thickness of 6 mm.

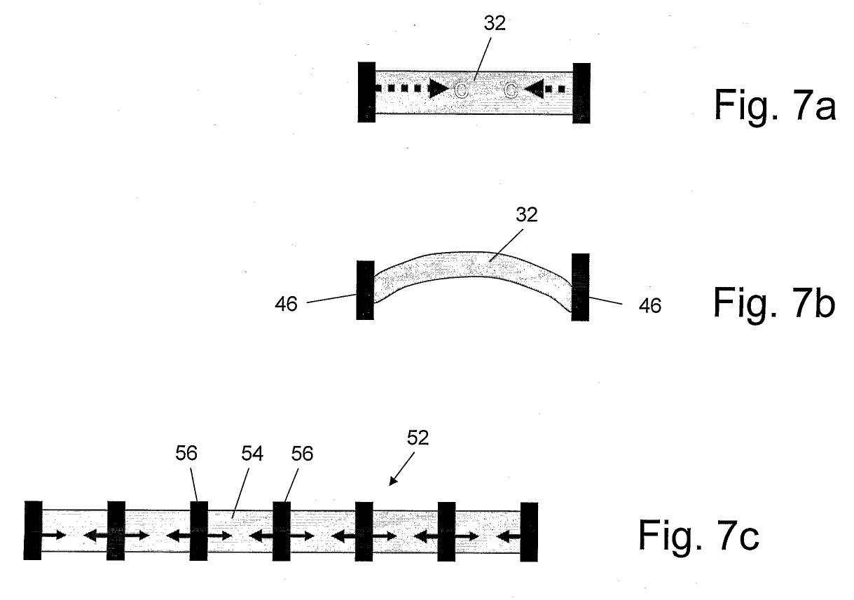

The interface element may be subjected to factory-produced compressive forces by being press fitted in a retaining member smaller than the size of the interface element, as schematically illustrated in FIGS. 6a-e. Before being compressed, interface element 32 shown in FIG. 6a is longer than retaining member 46 shown in FIG. 6c, which is provided with the cover element of the evacuation chamber. Interface element 32 is first elastically compressed, as shown in FIG. 6b, into temporary frame 48 defining an interior of smaller dimensions than the interior defined by retaining member 46. Temporary frame 48 is then inserted within retaining member 46, as shown in FIG. 6c, and then broken, as shown in FIG. 6d. Once temporary frame 48 is broken, interface element 32 slightly expands, abutting retaining member 46, as shown in FIG. 6e. Once interface element abuts retaining member 46, its length is shorter than its original dimension shown in FIG. 6a and therefore remains in a permanent compressed state.

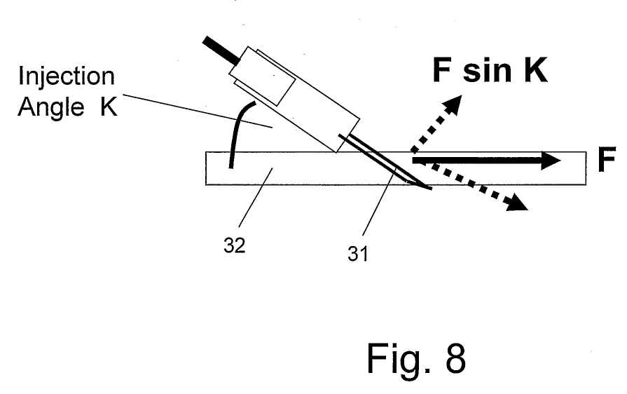

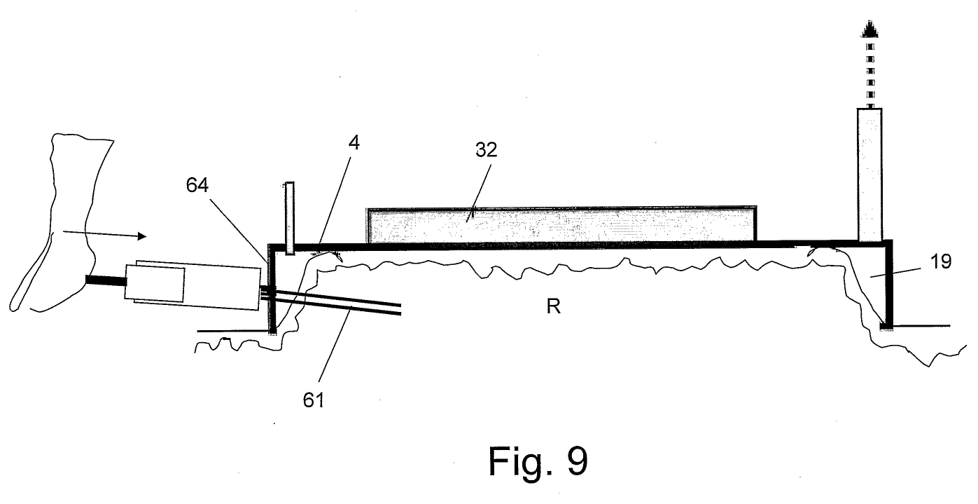

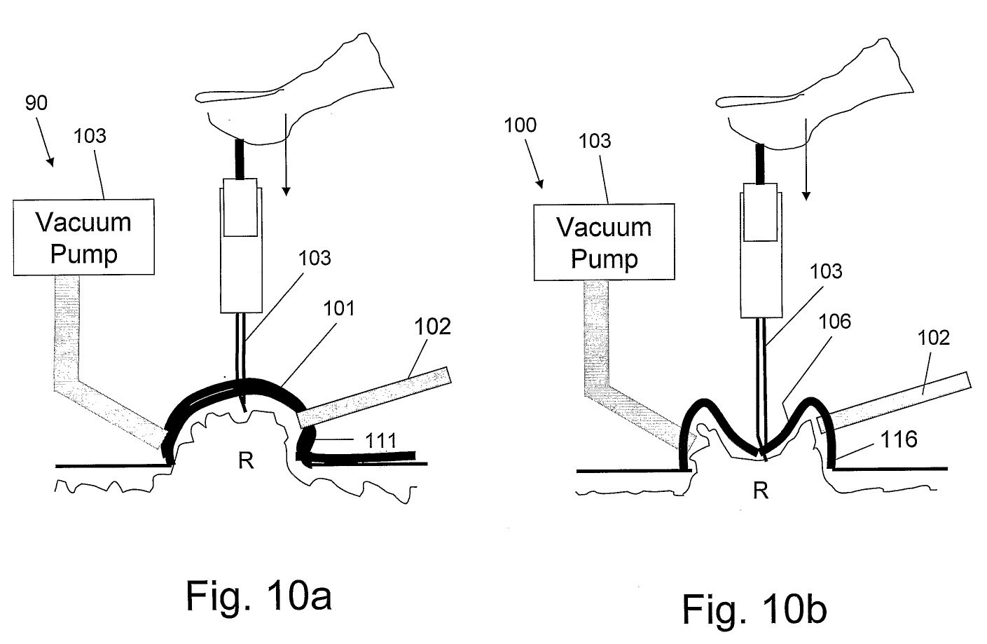

Due to the compressive forces C to which interface element 32 is subjected, as shown in FIG. 7a, the interface element has a tendency to assume a concave shape between its retaining element 46, as shown in FIG. 7b. The aspect ratio of interface element 32 is dependent upon the selected interface element material. The aspect ratio may be relatively high when a homogeneous material such as silicon is employed, and is generally low when a heterogeneous material such as cork is employed. Since interface element 32 tends to attain a minimal energy level corresponding to a decompressed state when its aspect ratio is relatively high, the interface element is liable to tear and to be unable to maintain a relatively high vacuum level when punctured by an injection needle, similar to the situation when it is not compressed and tensile forces increase the size of the puncture site, as shown in FIG. 4. To limit the aspect ratio of an interface element made of silicon, which is generally needed to maintain a sterile atmosphere above the target skin region of the medical treatment, a subdivided interface element 52 may be employed, as shown in FIG. 7c. Subdivided interface element 52 comprises a plurality of silicon sections 54, each of which is compressed between two dividing walls 56, e.g. made of polycarbonate. The dimensions of each section 54 are preferably small, e.g. 4 mm×4 mm×4 mm, to limit the degree to which each section is bent. During injection, the needle penetrates a silicon section 54. A subdivided interface element is unnecessary when the interface element is made from a heterogeneous material such as cork, melted grains of silicon, or rubber.

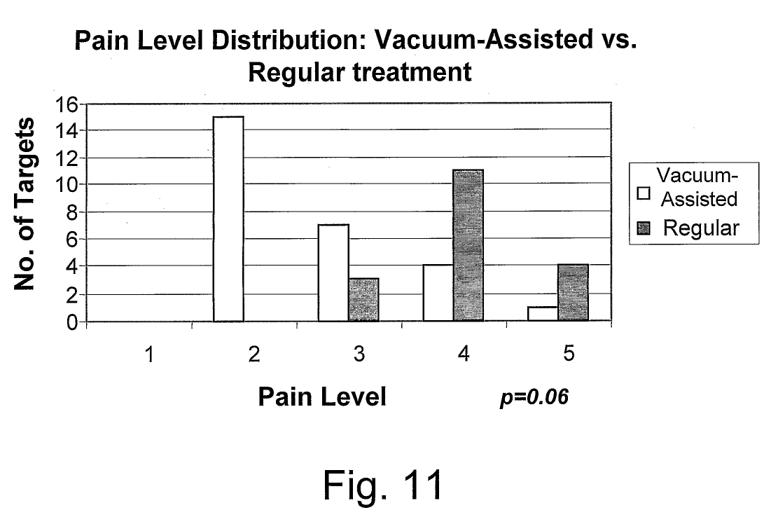

To prevent tearing of the interface element, injection needle 31 may be manipulated by the health professional administering the treatment such that it is disposed at a small injection angle K relative to a horizontal interface element 32, as shown in FIG. 8. At a small injection angle of K, the resultant tearing force normal to needle 31 is significantly reduced from F to FsinK, thereby minimizing the risk of structural failure to the interface element. The interface element will not tear when the injection angle is less than a predetermined value, which is dependent upon the strength of the interface material, its elasticity, and its threshold tearing force being a function of the aspect ratio of the interface element. The tear resistance of the interface element is increased when the target skin region is flattened by the interface element and thereby seals the puncture site within the interface element.

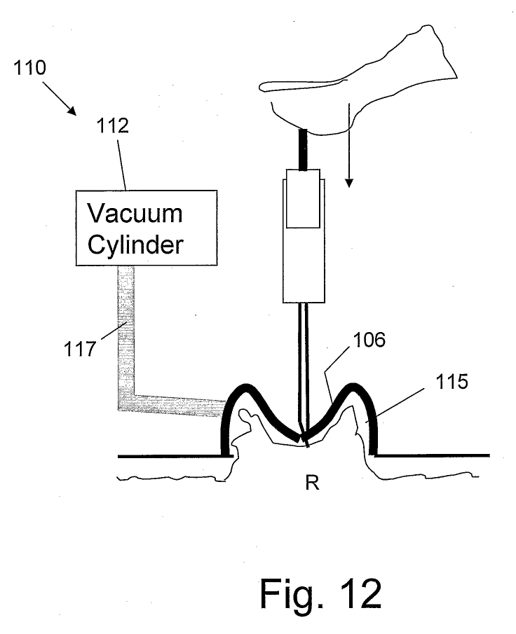

An elongated needle 61 may be injected into drawn skin region R through a cylindrical sidewall 64 of evacuation chamber 19, as shown in FIG. 9. When injection needle 61 penetrates through sidewall 64, an inwardly directed pressure-derived force acts on sidewall 64, compressing cover element 4 and interface element 32. In this injection method, interface element does not have to be subjected to any factory-produced compressive forces due to the pressure-derived self compression. A painless injection through a sidewall is advantageous for medical treatments such as the injection of collagen within the epidermis, essentially parallel to the skin surface, for wrinkle removal, a procedure which is normally very painful.

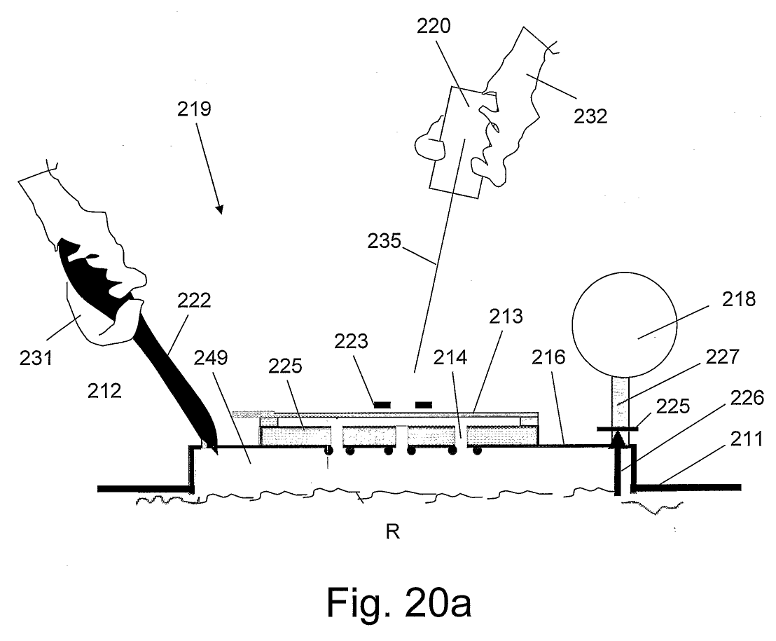

In another embodiment of the invention illustrated in FIG. 18, evacuation chamber 85 is configured as a slit defined by elongated, planar sidewalls 81 and 82 and by a rigid upper surface 83 extending between, and having a considerably shorter length than, sidewalls 81 and 82. For example, upper surface 83 may have a surface area of 10×50 mm and skin region R can be drawn to a height of 10 mm. After the vacuum pump is activated, skin region R is drawn through the interior of slit 85 and is compressed against surfaces 81-83. The compressing effect of skin region R is similar to a certain extent to that of skin pinching, although, as described hereinabove, the physiological reaction to vacuum generated compression is much different than the application of a positive pressure onto a skin surface. The injection needle is introduced through one of the sidewalls 81 or 82, which are puncturable.

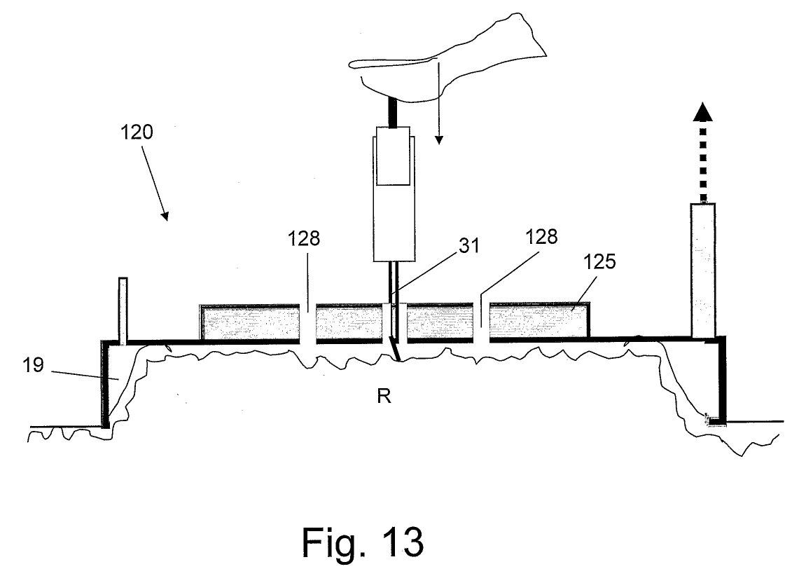

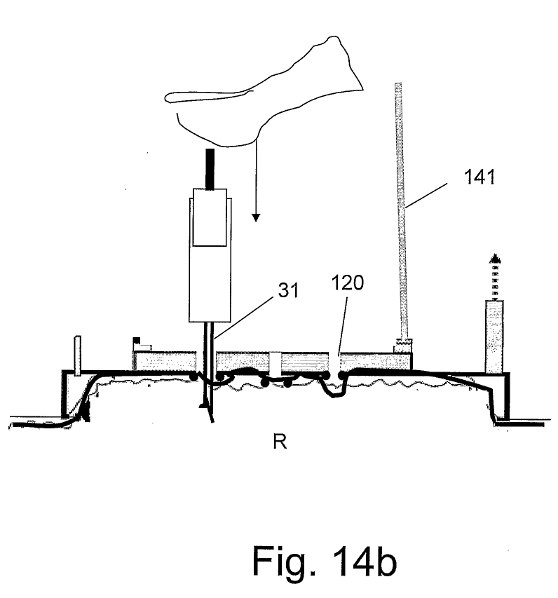

In another embodiment of the invention illustrated in FIGS. 10a-b and 12, the interface element is curvilinear, whether a convex interface element 101 shown in FIG. 10a or a concave interface element 106 shown in FIGS. 10b and 12. Convex interface element 101 shown in FIG. 10a is retained by a short, substantially vertical sidewall 111. Concave interface element 106 shown in FIG. 10b is retained by inwardly curved sidewall 116. A curvilinear interface element configuration is advantageous in that air introduced into the evacuation chamber via the puncture, which is caused by the injection of needle 103 into skin region R, due to the pressure differential between the air side and vacuum side of the evacuation chamber, will be vertically directed. Consequently the degree of compression of skin region R against the rigid interface element and the resistance to tearing of the interface element will be increased.

Apparatus 90 and 100 shown in FIGS. 10a and 10b, respectively, comprise vacuum pump 103 and a control unit (not shown) for generating the vacuum within the corresponding evacuation chamber prior to the injection and releasing the vacuum following the injection, in accordance with a selected sequence and in response to the vacuum level detected by pressure sensor 102.

Apparatus 110 shown in FIG. 12 comprises a pre-evacuated vacuum cylinder 112 for generating the vacuum within evacuation chamber 115 and means (not shown) such as a valve for isolating vacuum cylinder 112 from evacuation chamber 115. Evacuation chamber 115 may be disposable, and the means for isolating vacuum cylinder 112 therefrom may therefore be a breakable stop. Pressure sensor 102 (FIG. 10b) may also be employed to achieve a suitable pain alleviating vacuum level within evacuation chamber 115.

When skin contact sensor 221 (FIG. 2) is employed to detect the placement of evacuation chamber 115 on skin region R, the means for isolating vacuum cylinder 112 from evacuation chamber 115 may be automatically opened. Once skin contact sensor 221 detects the placement of evacuation chamber 115 on skin region R, the isolation means is automatically opened and a vacuum is applied to evacuation chamber 115 via conduit 117 in communication with vacuum cylinder 112. Since the opening of the isolation means, which is an operation generally not suitable for those of limited dexterity, may be automatically performed, and since evacuation chamber 115 need not be sanitized when a disposable evacuation chamber is employed, an injection may be painlessly self-administered. Since a vacuum pump is not needed, the apparatus is affordable, and therefore such an arrangement is particularly suitable for an insulin injection device which is used on a daily basis.

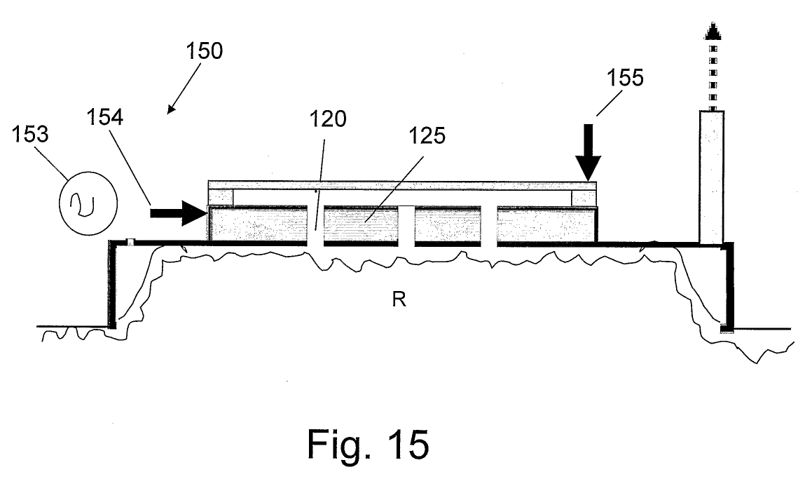

Apparatus 120 schematically illustrated in FIG. 13 comprises planar interface element 125 formed with a plurality of apertures 128 through each of which flattened skin region R may be injected. The use of an apertured interface element is advantageous in that, while substantially all of skin region R is flattened, the skin surface underlying each corresponding aperture 128 may be injected without contacting the bottom surface of interface element 125, so as to avoid a lack of biocompatibility to the material of interface element 125 or a lack of sterilization.

Apertures 128 need to be sufficiently small to ensure that the downwardly directed force, which is caused by air flow through the apertures as a result of the pressure differential of the air external and internal to the evacuation chamber, will be considerably smaller than the upwardly directed vacuum-generated force which urges skin region R to be in compressed relation with the bottom surface of interface element 125, in order to maintain the vacuum level within evacuation chamber 19. In order to ensure sufficient afferent inhibition, the applicant has found that the total surface area of the apertures should not be greater than 20% of the enclosed surface area of a planar interface element. When the interface element is curvilinear, as shown in FIGS. 10a-b, the upwardly directed vacuum-generated force will be increased and the infiltration of ambient air through the apertures will be proportionally decreased.

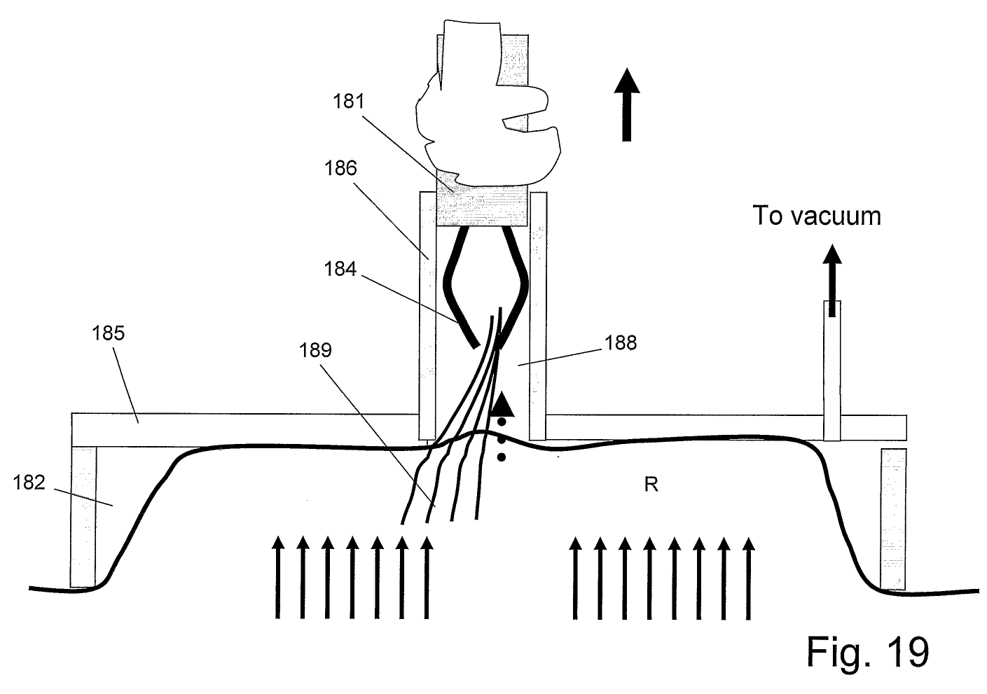

In the embodiment of FIG. 17, apparatus 130 is adapted to maintain the vacuum within evacuation chamber 139 and the resulting skin compression after the operation of the vacuum pump, or any other suitable vacuum generating means, has been terminated. Apparatus 130 comprises rim 132 of diameter H, which is connected to the underside of apertured interface element 125 and encircles aperture 120 through which a needle is introduced prior to injection into the skin region. Rim 132 may be an O-ring or may be integrally formed together with interface element 125.

As a vacuum is applied to evacuation chamber 139, an upward directed vacuum-derived compression force F is generated within the skin region, drawing the latter in compressed fashion towards apertured interface element 125. For sake of clarity, the skin region is shown to be subdivided into inner portion R_{I} underlying aperture 120 and outer portion R_{O} surrounding inner portion R_{I}. Due to the presence of rim 132, terminal surface 133 of inner portion R_{I} bordering with outer portion R_{O} abuts rim 132 and inner portion R_{I} is therefore prevented from contacting interface element 125. However, large-area interface engaging surface 135 of outer portion R_{O} is allowed to contact interface element 125, and therefore the entire skin surface of outer portion R_{O} is disposed above the entire skin surface of inner portion R_{I}. The skin surface of inner portion R_{I} remains in a substantially horizontal disposition due to the influence of the downward directed atmospheric pressure derived force P. Although inner portion R_{I} which is not compressed by interface element 125 is punctured by the injection needle, the pain sensation is inhibited by the afferent inhibition generated by the compression of outer portion R_{O} surrounding inner portion R_{I} onto interface element 125.

When a vacuum is applied to evacuation chamber 139 and interface engaging surface 135 of outer portion R_{O} is compressed against interface element 125, two volumes of negative pressure are produced within evacuation chamber 139: volume 142 enclosed by interface element 125, rim 132, and outer skin portion R_{O}, and volume 144 enclosed by interface element 125, sidewall 146 of evacuation chamber 139, and outer skin portion R_{O}. Even though rim 132 is subjected to a radial force P_{S} generated by the pressure differential between atmospheric pressure and the vacuum level within volume 142, rim 132 is not severed from interface element 125. The reactive force F_{R}, which is normal to skin surface 148 extending between rim 132 and interface engaging surface 135, applies a force onto rim 132 which counteracts radial force P_{S}, thereby ensuring the continued presence of volume 142.

Even after the termination of the vacuum generating means which induced the compression of interface engaging skin surface 135, a vacuum advantageously remains in volumes 142 and 144. Due to the presence of volumes 142 and 144, an upwardly directed vacuum-derived compression force F remains, although its magnitude is considerably less than when the vacuum generating means was operable, and interface engaging skin surface 135 continues to be compressed by interface element 125, although the area of interface engaging skin surface 135 is reduced as a result of the lower magnitude of compression force F. A vacuum remains in volumes 142 and 144 as long as the magnitude of upwardly directed compression force F is sufficiently great so as to ensure that outer skin portion R_{O} contacts both interface element 125 and rim 132. A significant parameter in determining the ability of apparatus 130 to maintain the vacuum within evacuation chamber 139 after the operation of the vacuum generating means has been terminated is diameter H of rim 132. If diameter H of rim 132 is excessively small, outer skin portion R_{O} will contact interface element 125 and rim 132 for a shorter period of time. For example, the inventors have determined that a rim diameter of 0.7 mm is able to maintain a vacuum level of 0.5 atm for a duration of over 1 minute.

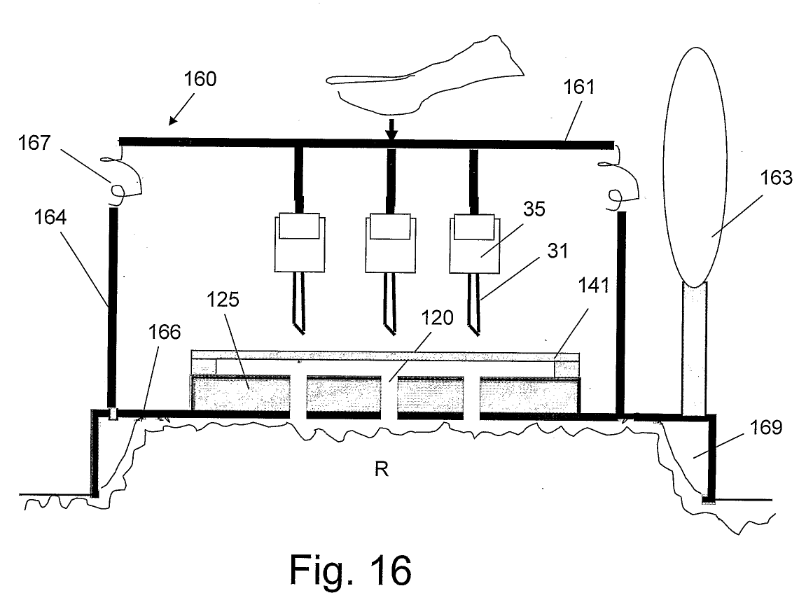

In the embodiment of FIGS. 14a-b, apparatus 140 is adapted to minimize the infiltration of ambient air through apertures 120 of interface element 125 by employing shield element 141. Shield element 141 shown in FIG. 14a may be placed directly on top of interface element 125, or alternatively seal element 143 may be interposed between shield element and interface element 125. By covering interface element 125 with shield element 141 prior to applying the vacuum, infiltration of ambient air through apertures 120 is minimized and the vacuum level within evacuation chamber 149 can be consequently increased. Shield element 141 may be a thin sheet of polymer, such as cellophane, mylar, Kapton®, and nylon, or may be cloth suitable for sterile packing and bandages, and its thickness may range from 10-50 microns. After the vacuum is applied and skin region R is drawn and compressed against interface element 125, shield element 141 is removed or peeled as represented by arrow 147 in preparation of a skin injection while the generated vacuum is maintained due to the small size of apertures 120. In FIG. 14b, needle 31 is shown to be injected into skin region R, after shield element 141 has been peeled and needle 31 has been introduced into a selected aperture 120.

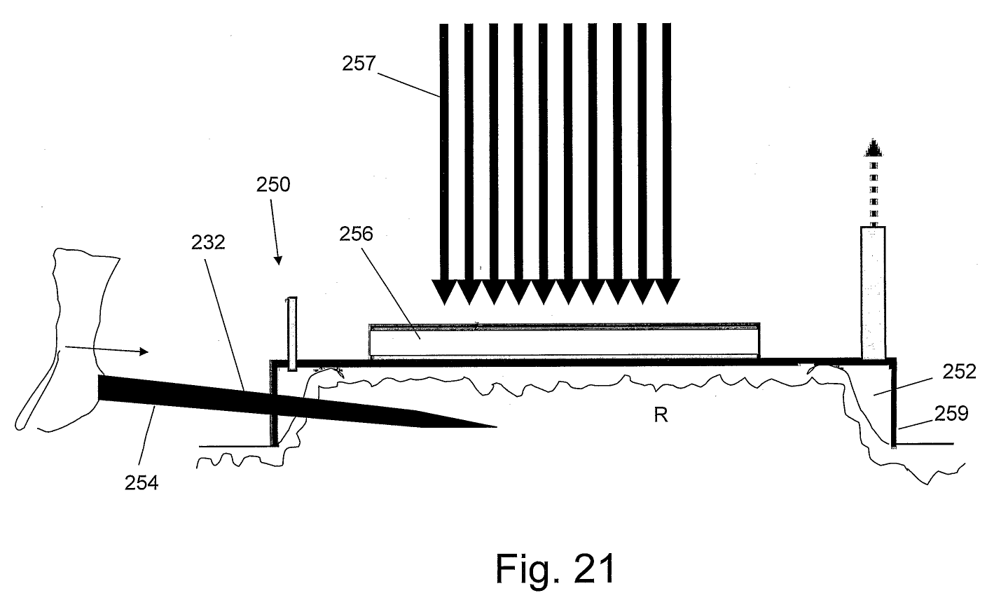

In the embodiment schematically illustrated in FIGS. 20a-b, which may be considered the preferred embodiment of the present invention, apparatus 219 enables a single health professional to position evacuation chamber 249 into which skin region R is drawn prior to the medical treatment, generate the vacuum therein, and administer the injection. FIG. 20a illustrates the positioning of evacuation chamber before the application of vacuum therein and FIG. 20b illustrates the administration of an injection after a skin region is drawn by the vacuum applied to the evacuation chamber.

Evacuation chamber 249 has a rigid planar cover element 216, the surface area of which is greater than the threshold pain inhibiting area. Cover element 216 may be made of polycarbonate or any other rigid polymer which can be sterilized in ethylene oxide or by means of radioactive irradiation. Apertured interface element 225 is retained within cover element 216 and is covered by thin, adhesive and puncturable shield element 213, e.g. the Tegaderm™ HP Transparent Dressing produced by 3M, USA. Marks 223 may be indicated on the upper face of shield element 213, to assist in directing injection needle 235 to apertures 214 formed in interface element 225. Rims 229, e.g. an O-ring, may be added to the underside of rigid cover element 216 in maintain the vacuum once the injection needle pierces shield element 213. Cover element 216 may be formed with an integral rim protruding from the underside thereof. To position evacuation chamber 149 above skin region R, handle 222 connected to cover element 216 of the evacuation chamber is held by a first operator hand 231.

Apparatus 219 employs vacuum source 218, which is embodied by a spherical container. Air is evacuated from vacuum source 218 to a relatively high vacuum level, e.g. 50 millibar, and conduit 227 connected to cover element 216 and extending from vacuum source 218 to evacuation chamber 249 is sealed. The volume of vacuum source 218 is sufficiently large to induce fluid flow thereto at a relatively high rate from evacuation chamber 249, when conduit 227 is in fluid communication with evacuation chamber 249, so that the generated vacuum level will be greater than the threshold pain inhibiting level. The volume of vacuum source 218 should be at least twice the volume of evacuation chamber 249. During tests conducted by the applicant, a high level of pain inhibition was sensed when vacuum source 218 was five times the volume of evacuation chamber 249.

In one embodiment, pins 226 located below vacuum source 218 are used to allow conduit 227 to be in fluid communication with evacuation chamber 249. After vacuum source 218 is evacuated and sealed by rubber membrane 225 stretched across the interior of conduit 227, the latter may be opened by perforating membrane 225. By placing a pin 226 on skin region R and below conduit 227, the pointed end of pin 226 perforates membrane 225 as evacuation chamber 249 is lowered and placed on skin region R. This configuration facilitates reuse of the apparatus. Following injection of beneficial material into one skin region and release of the vacuum, evacuation chamber 249 may be repositioned to another skin region. After a new membrane 225 is resiliently stretched and inserted within a suitable slit formed in conduit 227, the interior of vacuum source 218 may be evacuated through a valve in communication therewith (not shown) and by means of an external vacuum pump.

Once evacuation chamber 249 is lowered on skin region R and a vacuum is generated by means of vacuum source 218, skin region R is drawn and compressed against cover element 216 within a short period of time, e.g. less than 0.5 second. A fast and reliable vacuum generating capability is of great importance to patients and to health professionals operating the apparatus, to ensure pain inhibition. The vacuum level within evacuation chamber 249 and sterility of skin region R are increased by using thin sheet 211 surrounding evacuation chamber 249 and shield element 213 covering apertured interface element 225. Pain inhibition is made possible by employing an evacuation chamber 249 having a relatively large skin engaging area, e.g. the total area of cover element 216 and the bottom of interface element 225 is 20×40 mm, in order to gate pain nerves by transmitting pressure signals to the dorsal horn through the pressure nerves following compression of a sufficiently large enough number of pressure receptors, regardless of the pain level that would be generated by injector 220 if apparatus 219 were not employed.

Needle 235 is preferably injected in skin region R within approximately 2 seconds following generation of the vacuum within evacuation chamber 249 since the duration of pain inhibition is limited by a period of approximately 3 seconds following generation of the vacuum. While only a limited number of injector types may be used in conjunction with prior art vacuum-assisted injection devices, any commercially available injector may be employed in conjunction with the apparatus of the present invention. Nevertheless, the selected injector should be able to inject beneficial material into skin region R within 2 seconds, as explained hereinabove. Marks 223 assist the health professional administering the injection to properly position needle 235 over an aperture 214 prior to the injection.

Apparatus 219 is preferably provided with means for automatically releasing the vacuum from evacuation chamber 249, as shown in FIG. 20b. The vacuum release means is adapted to release the vacuum from evacuation chamber 249 at a predetermined interval following generation of the vacuum therein. Consequently, injection needle 235 may be retreated and evacuation chamber 249 may be repositioned to another skin region after injection. The vacuum may be released by means of mechanism 228 and a spring loaded plunger 233, e.g. connected to sheet 211. Plunger 233 is actuated upon placement of evacuation chamber 249 on skin region R and is caused to extend within mechanism 228. As plunger extends within mechanism 228, a valve (not shown) in communication with which both conduit 227 and the surrounding ambient air A is opened by means of a suitable gear train within a short period of time, e.g. 3-4 seconds. Alternatively, the vacuum release means may be embodied by electrically operating components, such as skin contact sensor 221 (FIG. 2) and a valve actuator of mechanism 228, which is in electrical communication with skin contact sensor 221.

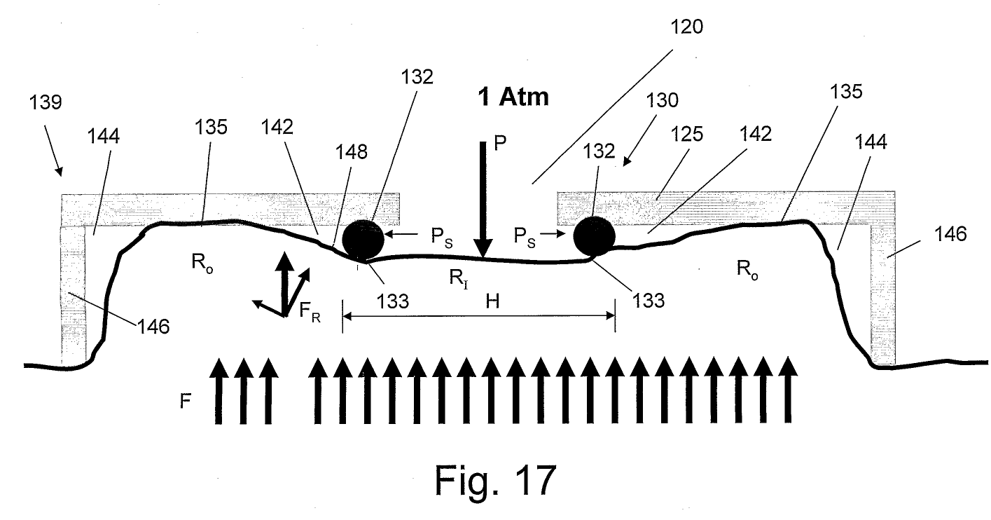

In the embodiment schematically illustrated in FIG. 15, apparatus 150 comprises a vibrator 153 driven by a miniature motor or by a small AC electromagnet, e.g. one manufactured by Vibraderm Inc, USA. Vibrator 153 is kinematically connected to apertured interface element 125 such that the generated vibrations may be horizontally directed as shown by arrow 154 or vertically directed as shown by arrow 155. Vibrator 153 may be operated both before and during generation of the vacuum. The vibration frequency ranges between 5-100 Hz and the vibration amplitude ranges between 0.1-1 mm. The selected vibration amplitude is preferably dependent on the configuration of the evacuation chamber. For example, if the injection needle is introduced through apertures having a diameter of 1 mm, the generated vibrations may have an amplitude of 0.2 mm. Since vibrations may contribute to the afferent by generating pressure signals in pressure receptors of the skin, the generated vacuum level may be as low as 200 mmHg.

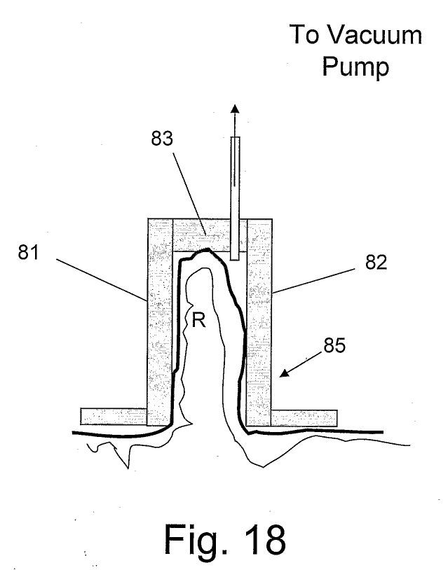

FIG. 16 illustrates an arrangement for automatically administering a plurality of injection needles. Apparatus 160 comprises horizontal bar 161 for holding a plurality of needle applicators 35 therebelow and a guide track 164 perpendicular to bar 161. Guide track 164 is connected to, or integrally formed with, cover element 166 of evacuation chamber 169. Engagement means (not shown) are provided for coupling bar 161 to guide track 164 to ensure proper alignment of each injection needle 31 with respect to an underlying aperture 120 of interface element 125, which is provided with a removable shield element 141. Actuation means (not shown) are provided to lower bar 161, in synchronization with the application of the vacuum to evacuation chamber 169, so that each needle 31 will be introduced through corresponding aperture 120 and be injected within skin region R. Apparatus 160 may be disposable and may employ a vacuum cylinder 163 having a considerably larger volume than that of evacuation chamber 169, e.g. a volume 10 times as great as the volume of evacuation chamber 169. Vacuum cylinder 163 may be activated by breaking breakable stop or by means of a skin contact detector, such as a microswitch or an optocoupler (not shown). Bar 161 may be spring biased by springs 167 to be raised above interface element 125 prior to injection.

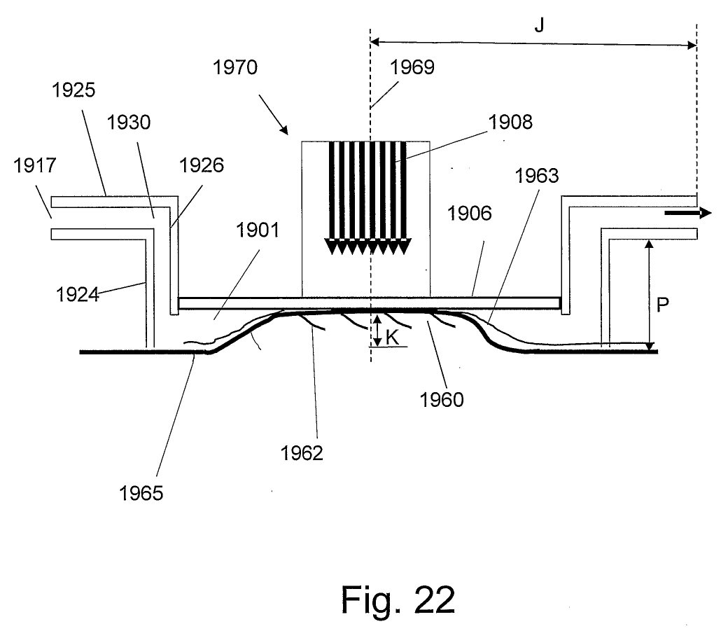

In the embodiment of FIG. 21, apparatus 250 employs an evacuation chamber 252 by which a dual light and needle based medical treatment is painlessly administered. A dark, e.g. black, elongated needle 254 is inserted within the epidermis and substantially parallel to the skin surface in an identical way as injectors for collagen filling or for wrinkle reduction are introduced. However, needle 254 does not serve as an injector but rather as a thin blackbody, capable of attracting the optical energy of a laser or IPL beam 257 directed under the skin surface and consequently thermally damaging its surroundings. One suitable application for this apparatus is the contraction of wrinkles present in the vicinity of, and overlying, needle 254.

In order to inhibit the pain which is normally sensed during these types of treatment, a vacuum is generated within evacuation chamber 252. Evacuation chamber 252 comprises transmitting element 256 which is transparent to beam 257, to allow the latter to subcutaneously propagate to skin region R and heat needle 254, and puncturable sidewalls 259 so that needle 254 may penetrate the interface element and the adjoining skin region R. Needle 254, which may be produced from medical stainless steel galvanized in a black color having a diameter ranging from 50-1000 microns and a length of approximately 30 mm, is painlessly introduced through sidewall 259 to skin region R after a vacuum, e.g. of greater than 400 millibars, has been applied to evacuation chamber 252 and skin region R has been compressed by the underside of transmitting element 256 greater is size than the pain inhibiting threshold, e.g. 12×40 mm. Following introduction of needle 254 into skin region R, light beam 257 having an energy density ranging from 10-100 J/cm² is fired and the resulting photothermolysis effect is painless. A suitable laser for generating beam 257 is an Nd:YAG laser which produces a pulse duration of 10-300 millisec. The generated light is well absorbed in artificially blackened needle 254 and creates minimal damage to the skin. The blood expulsion caused by the compression of skin region R ensures that the heat conducted from the heated needle is transferred to the adjacent collagen fibers rather than to blood.

Pain Inhibition During Light-Based Treatments

When a evacuation chamber is placed on a skin target, the apparatus provides an additional advantage in terms of the capability of alleviating pain that is normally caused during e.g. the treatment of hair with intense pulsed monochromatic or non-coherent light.

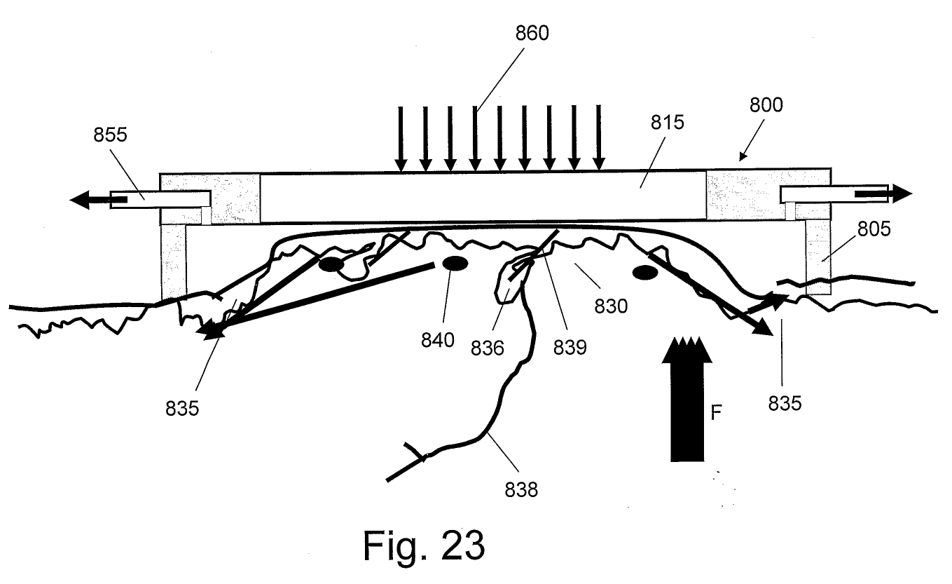

As shown in FIG. 22, apparatus 1970 is configured so as to bring skin target 1960, when a vacuum is applied, in contact with transmitting element 1906, e.g. made from sapphire, which is secured to the proximate end of evacuation chamber 1901. The Applicant has surprisingly discovered that the immediate sharp pain which is normally sensed during a light-based skin treatment is alleviated or eliminated when a skin target contacts, and is flattened by, the transmitting element. The level of the applied vacuum is suitable for drawing skin target 1960 towards evacuation chamber 1901 by a slight protrusion of K, e.g. 2-4 mm, with respect to adjoining skin surface 1965, a distance which is slightly greater than the gap between transmitting element 1906 and the distal end of outer wall 1924 of evacuation chamber 1901. During generation of pulsed beam 1908 from any suitable intense pulsed laser or light source propagating through transmitting element 1906, whereby e.g. hair follicles 1962 located under the epidermis of skin target 1960 are treated by the generated optical energy, skin target 1960 is drawn to be in contact with transmitting element 1906. As skin target 1960 is drawn by the vacuum into evacuation chamber 1901 and contacts transmitting element 1906 by means of the resulting proximally directed force, the pain signals generated by the nervous system during the heating of hair follicles 1962, or of any other suitable targeted skin structure, of the patient are inhibited. Accordingly, the synchronization of an optimal delay between the application of the vacuum and firing of the light treatment pulse is a key factor in pain reduction, in order to ensure that skin target 1960 is in contact with transmitting element 1906 for a sufficiently long nerve inhibiting duration when pulsed beam 1908 is fired. Pain reduction is noticeable with use of this apparatus even when when the energy level of the light directed to skin target 1960 is increased, an effect which normally causes an increase in immediate sharp pain.

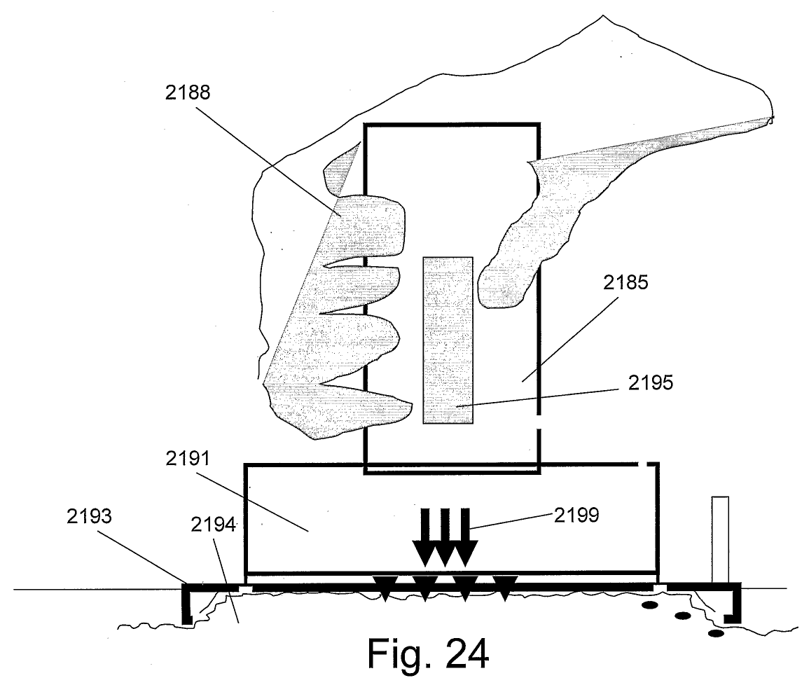

Evacuation chamber 800 illustrated in FIG. 23 is also configured to alleviate the pain resulting from the firing of light beam 860 onto skin target 830. When a vacuum is applied onto evacuation chamber 800 via conduits 855, skin target 830 is drawn and contacts transmitting element 815. Instead of sensing immediate sharp pain during impingement of each treatment pulse with a skin area 836 of skin target 830, the magnitude of proximally directed force F resulting from the applied vacuum causes nerve 838 surrounding a corresponding hair bulb and extending to skin area 836 to be pressed onto transmitting element 815 for a sufficient duration to inhibit the pain sensation. Light beam 860 is of a wavelength which is well absorbed by hair follicles 839. By optimizing the time delay between application of the vacuum and the firing of light beam 860, the pain sensation is sufficiently inhibited and the energy density of light beam 860 need not be decreased.

The apparatus for alleviating pain during vacuum-assisted light-based treatments of the skin may include a control device (not shown) for adjusting the vacuum level generated by the vacuum pump, as well as the time delay between the application of the vacuum and the firing of light beam. The control device preferably has a plurality of finger depressable buttons, each of which is adapted to set the vacuum pump and light source at a unique combination of operating conditions so as to generate a predetermined vacuum level within evacuation chamber 800 and to result in a predetermined time delay between the operation of the vacuum pump and the firing of light beam 860, and a display to indicate which button was depressed. The apparatus may also comprise control valves in electrical communication with the control device for evacuating air into evacuation chamber during a vacuum applying mode and for introducing air therein during a vacuum release mode, respectively. The health professional is aware of the anticipated pain level that a patient generally senses when one of these buttons is depressed. If the pain threshold of a patient is relatively low or if the application of the vacuum by the evacuation chamber onto the skin target is annoying, the health professional may change the combination of operating conditions by depressing a different button. Alternatively, the pain threshold of a patient may be objectively determined by an electrical measurement of a muscle reflex in response to pain.

As skin target 830 is pressed onto transmitting element 815 during the application of the vacuum, blood is displaced from skin target 830 to peripheral skin area 835. Although the blood fraction volume in peripheral skin area 835 is increased, the latter is nevertheless liable to be damaged by the treatment light, which may diffuse subcutaneously from skin target 830 to skin area 835. To counteract the potential thermal injury to skin area 835, heat absorbing gel (not shown in the figure) is applied to skin target 830 prior to application of the vacuum and is subsequently squeezed to peripheral skin area 835 by means of transmitting element 815. The displaced gel therefore advantageously protects peripheral skin area 835 from being injured by subcutaneously diffused treatment light.

Apertured interface element 125 shown in FIG. 13 is also useful when the medical treatment is administered solely by means of a laser. A normally painful sensation will be inhibited if the vacuum level applied to evacuation chamber 19 is sufficiently high, e.g. 400 mmHg and the surface area of interface element 125 is sufficiently high, e.g. 15×25 mm. Apertures 128 are advantageous in that the generated laser beam can propagate therethrough in order to impinge on skin region R when not able to be transmitted through the material from which interface element 125 is composed. When Ruby Q-switched, frequency doubled Nd:YAG, Nd:YAG, or Alexandrite lasers having an energy density ranging from 4-12 J/cm² and a pulse duration ranging from 1-20 nanosec are employed, for example, for the removal of tattoos or the treatment of pigmented lesions, the transmitting interface element is liable to shatter or a coating applied to the interface element is liable to decompose. However, when the laser beam is directed through an aperture 128 having a diameter of approximately 4 mm, the optical energy of the laser beam will not be absorbed within the interface element. Other lasers that may be operated in conjunction with an apertured interface element are ablative lasers such as a CO₂ or Erbium laser.

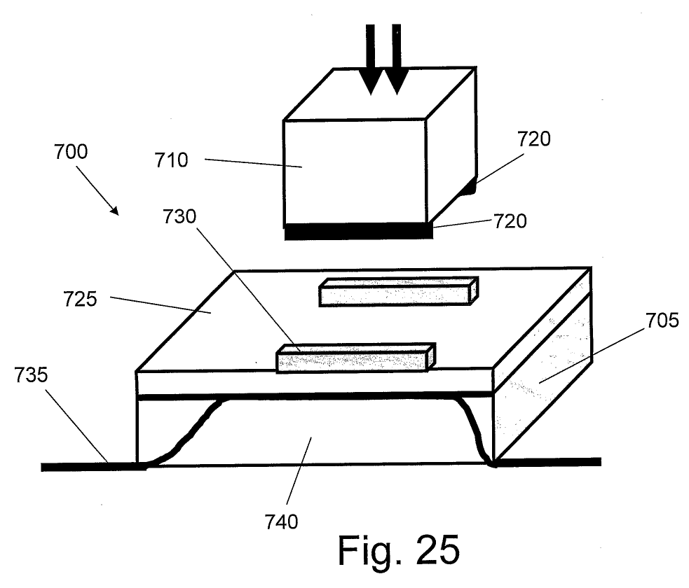

FIG. 24 illustrates a treatment handpiece 2185 which comprises a light source 2195 and is held by a hand 2188. The treatment light 2199 propagates through transmitting element 2191 and pain inhibiting evacuation chamber 2193, which draws and flattens skin 2194 in order to inhibit the transmission of pain.

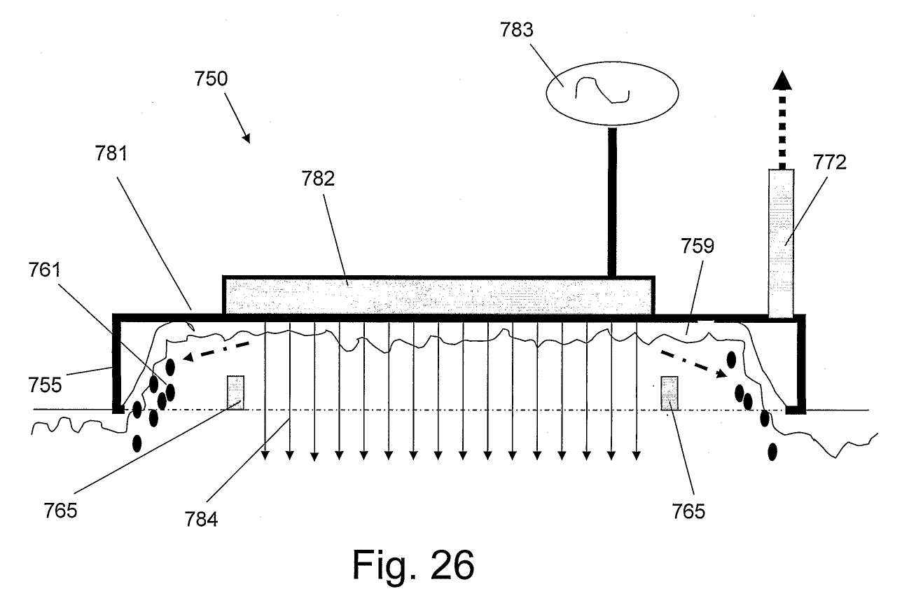

FIG. 25 illustrates another embodiment of the invention which is suitable for pain alleviation. Apparatus 700 comprises evacuation chamber 705 and IPL treatment light source 710, e.g. one produced by Syneron USA, which is provided with an RF source at the distal end thereof in the form of two electrodes 720. When transmitting element 725 of evacuation chamber 705 is made of sapphire, which has electrical insulating properties, the RF waves are prevented from propagating to skin target 735. To allow sapphire to be a suitable transmitting element for apparatus 700, two metallic conducting electrodes 730 are welded in two slits, respectively, formed in the sapphire transmitting element 725. The slits in sapphire transmitting element 725 may be formed by ultrasonic drilling or by precision abrasive drilling, such as with bits produced by American Precision Dicing Inc, USA, Rotem, Israel, or KPE, Israel. Exemplary dimensions of the electrodes are a width of 2 mm, a length of 17 mm long, and a depth of 2 mm deep, so as to be compatible with a diode laser such as produced by Syneron so that the electrodes of the diode laser may be placed on electrodes 730 of the sapphire transmitting element. Electrodes 730 are positioned to be within the propagation path of electrodes 720 integrally formed in light source 710. Suitable means, such as a magnetic rod (not shown), may be used to ensure the quick centering of light source 710 with respect to electrodes 730 of sapphire transmitting element 725. During application of the vacuum, skin target 740 contacts the sapphire transmitting element 725 and electrodes 730 transmit RF waves to skin target 740.

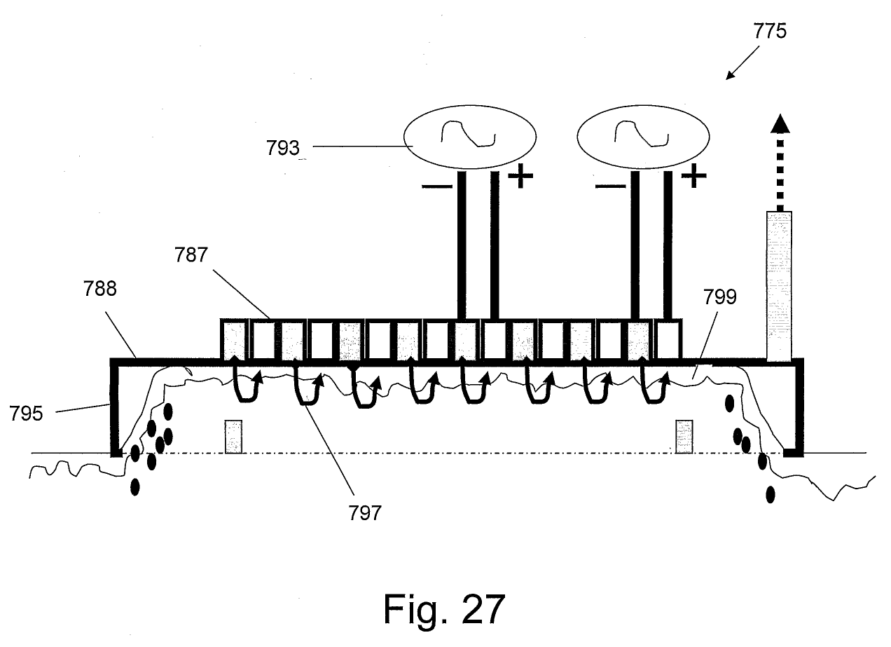

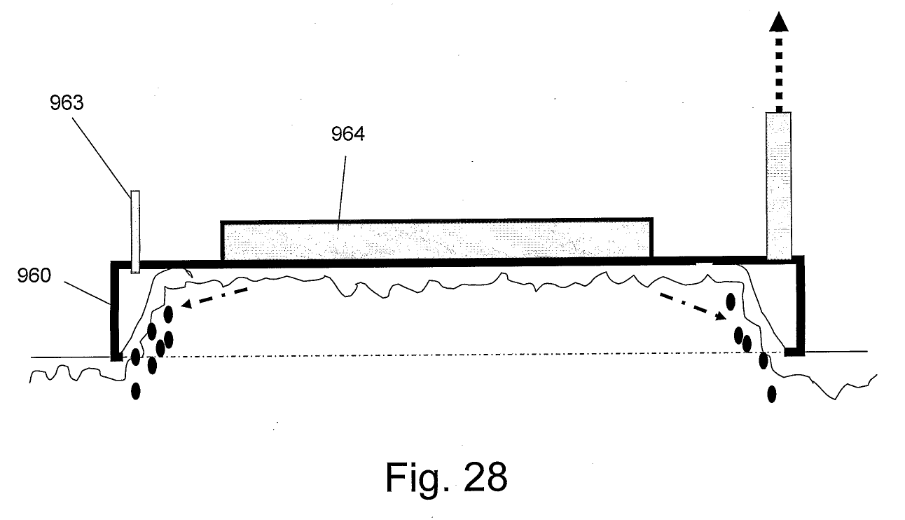

FIGS. 26 and 27 illustrate another embodiment of the invention wherein a large sized evacuation chamber is used for pain alleviation in conjunction with a RF-based skin treatment. The apparatus of FIG. 26 employs a monopolar RF source, while the apparatus of FIG. 27 employs a bipolar RF source. Each of these RF sources is used for different types of treatment. A monopolar RF source is generally employed when deep skin tightening is needed, such as for skin of the abdomen or legs with cellulite. A bipolar RF source is generally employed for more superficial skin tightening such as with respect to facial treatments. If so desired, the RF-based skin treatment may be supplemented by a light-based treatment.

As shown in FIG. 26, apparatus 750 comprises RF source 783, evacuation chamber 755, evacuation chamber cover 781, and transmitting element 782 positioned within evacuation chamber cover 781. Air is evacuated through duct 772 during the generation of a vacuum within chamber 755. Markers 765 located on a side of evacuation chamber 755 and separated by a distance substantially equal to the length of transmitting element 782 assist in the relocation of the evacuation chamber to a desired position while displacing the handpiece containing the evacuation chamber from one skin target to another. By being sufficiently conspicuous, markers 765 provide a visual association with the location of the previous skin target.

Transmitting element 782, which is capable of being in contact with drawn skin 759, may be made from a transparent material coated with a transparent conductive coating, such as produced by Edmund Optics Inc., USA, Melles Griot Inc., USA, or Ophir Optics, Inc., USA, or may be a metallic element. Transmitting element 782 is able to conduct monopolar field 784 generated by RF source 783 through drawn skin 759. Monopolar field 784, which may be generated at an energy density ranging from 1 J/cm² to 50 J/Cm² and a frequency ranging from 0.4 MHz to 1 GHz, is perpendicular to the surface of drawn skin 759 and terminates at a return electrode placed on a bodily portion such as the back, as well known to those skilled in the art. For example, monopolar field 784 may be generated at an energy density of 2.4 J/cm² and a frequency of 2.4 MHz.

Evacuation chamber 755 is configured to induce blood expulsion from the skin target when a vacuum is applied within evacuation chamber 755 above the the skin target. When blood 761, which has relatively low electrical resistance, is expelled in response to the generation of a vacuum of approximately 100 torr, waves of RF energy 783 are able to propagate through the connective tissue or the fatty tissue therebelow of drawn skin 759, rather than being directed through the blood vessels if blood were not expelled. The path of minimal resistance for the flow of electrical current of RF field 784 is therefore not directed through the expelled blood 761, but rather through the connective tissue perpendicular to the upper skin surface. The large proportion of RF energy 783 which is absorbed within drawn skin 759 is able to uniformly heat the collagen-rich reticular dermis and promote skin contraction for the removal of wrinkles. Depending on the depth penetration, which is a function of the frequency of RF source 783 as well known to those skilled in the art, RF field 784 may impinge upon the cellulite or fat level which is disposed below the reticular dermis and cause skin contraction at the cellulite depth or the softening of fat. When a higher-level vacuum of approximately 400 torr is generated, pain signals are inhibited and the treatment is painless.

FIG. 27 illustrates apparatus 775 which comprises an evacuation chamber 795 that is suitable for effecting vacuum-assisted treatments in conjunction with a bipolar RF source 793. An array of electrode pairs 787 suitable for inducing bipolar field 797 generated at a frequency ranging from 0.2-4 MHz is positioned on the cover 788 of evacuation chamber 795, and the number of electrode pairs 787 may vary from 1 to 100, depending on the size of cover 788 and the depth of treatment. A bipolar field 797 generated at an energy density of 30 J/cm² and a frequency of 450 KHz is suitable. Cover 788 may be opaque to monochromatic light when RF source 793 is the sole source of energy that is used for the treatment of a skin disorder. Cover 788 may be transparent to monochromatic light when a skin treatment is effected by means of bipolar RF source 793 in addition to a pulsed light source

Evacuation chamber 795 is adapted to expel blood to the periphery thereof, and the connective tissue within drawn skin target 799 is therefore able receives the majority of the energy of RF field 797, which normally would be diverted to the blood vessels located with skin target 799 constituting paths of least electrical resistance without influence of the blood expelling evacuation chamber, so as to achieve an efficacious treatment. A prior art treatment, such as one conducted by Syneron, Israel which utilizes the blood flow path in order to heat portions of the tissue, as explained by N. Sadick et al, “Selective Electro-Thermolysis in Aesthetic Medicine: A Review”, Lasers in Surgery and Medicine 34:91-97 (2004), is not capable of inhibiting pain by the skin flattening technique of the present invention. Similarly, a prior art technique carried out by means of the Aluma produced by Lumenis, USA, and described by M. Goldman in “Treatment of Wrinkles and Skin Tightening using Bipolar Vacuum-Assisted Radio Frequency Heating of the Dermis”, Lumenis, whereby skin is drawn in response to a small vacuum level of 28 mmHg between two parallel electrodes parallel to the skin is not capable of inhibiting pain by the skin flattening technique of the present invention.

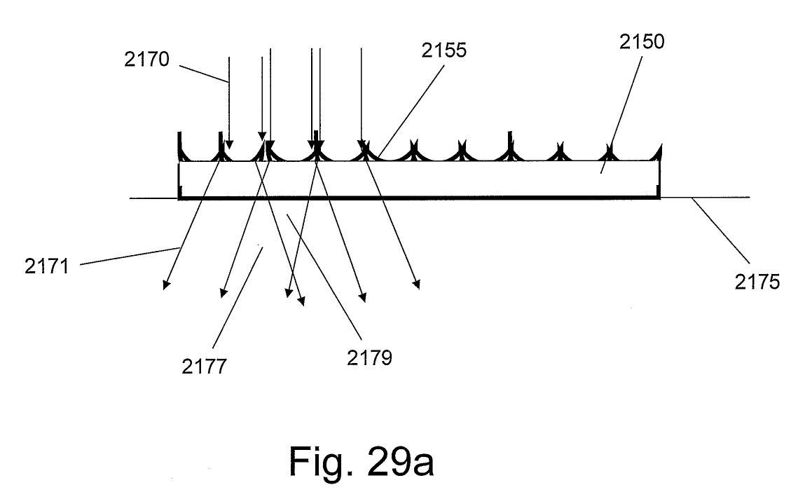

FIG. 28 illustrates an evacuation chamber 960 which is suitable for a pain inhibiting dermatological treatment by means of an electromagnetic source applied through transmitting element 964. Evacuation chamber 960 is provided with pressure sensor 963 for measuring the air pressure therewithin, so as to determine whether the applied vacuum level is sufficient to inhibit the transmission of pain signals. Pressure sensor 963 may also be used in a closed loop control system whereby the vacuum pump speed is varied in response to the detected vacuum level, in order to achieve a desired level of pain inhibition. The operator normally sets the target pressure level within evacuation chamber to a value ranging between 400-600 mmHg.

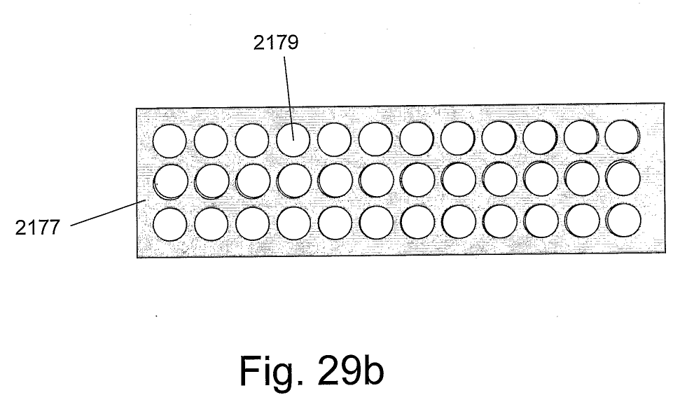

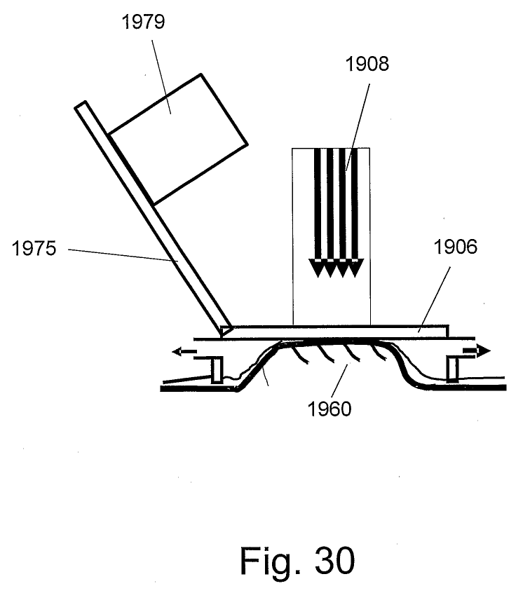

FIGS. 29a and 29b illustrate an additional embodiment of the present invention wherein an array of divergent lenses is provided, for an improved rate of healing for tissue that has been treated by laser treatment light. The relatively high vacuum level that is generated in order to achieve pain inhibition provides an additional advantage in terms of limiting the degree of scattering by the treatment light. If a relatively high vacuum level were not generated within the evacuation chamber, the treatment light would be scattered to a greater degree by the molecules and collagen bundles within the skin, and an array of divergent lenses would further increase the degree of scattering so that the treatment light would not be efficacious.

As shown in FIG. 29a, the proximal face of transmitting element 2150 of the evacuation chamber has an array of small concave lenses 2155. Lenses 2155 are divergent so that treatment light 2170 which is substantially perpendicular to skin surface 2175 generates ray of light 2171 that are oblique with respect to skin surface 2175. Due to the divergence of exit rays 2171, regions of higher energy density 2177 resulting from constructive overlap of the exit rays and regions of lower energy density 2179 resulting from the lack of overlap of the exit rays are produced. Transmitting element 2150 is advantageous in that a skin target underlying regions of lower energy density 2179 achieve a faster rate of healing due to the reduced thermal damage thereat. On the other hand, increased treatment efficacy is achieved in regions of higher energy density 2177.

FIG. 29b schematically illustrates in plan view the energy distribution of the treatment light transmitted through the array of lenses 2155 onto the underlying skin surface. The regions of lower energy density 2179 are shown as white circles, and the regions of higher energy density 2177 are shown are shown as grey regions surrounding a corresponding white circle.

The diameter of lenses 2155 may vary from 0.5 mm to 3 mm. The negative focal length may be 1-5 times the diameter of the lens. The array is a dense array, such as a hexagonal array of lenses arranged such that each lens is tangential to six adjacent lenses. For 1-mm diameter lenses, the lens density is approximately 1 lens/mm². Lenses 2155 may be produced from plastic, glass or sapphire and purchased from a large number of lenslet array manufacturers. They may also be produced as a holographic element from HoloOr Ltd., Israel.

An array of lenses 2155 is particularly suitable for skin tightening. When a laser beam generated by an Alexandrite laser having a wavelength of 755 nm or generated by an Nd:YAG laser having a wavelength of 1064 nm wavelength is transmitted through transmitting element 2150 into the flattened skin, the skin target from which blood vessels have been expelled supports a deeper penetration of light and a larger absorption thereof by collagen. Another suitable laser is one identical to a laser produced by DDC Technologies, Inc., USA. Each of these lasers may be operated for a duration of 0.5-5 seconds in order to heat the skin to a temperature of approximately 55° C. at a depth of approximately 1-2 mm. The average laser power is 80 W and the energy density is approximately 15-50 J/cm².

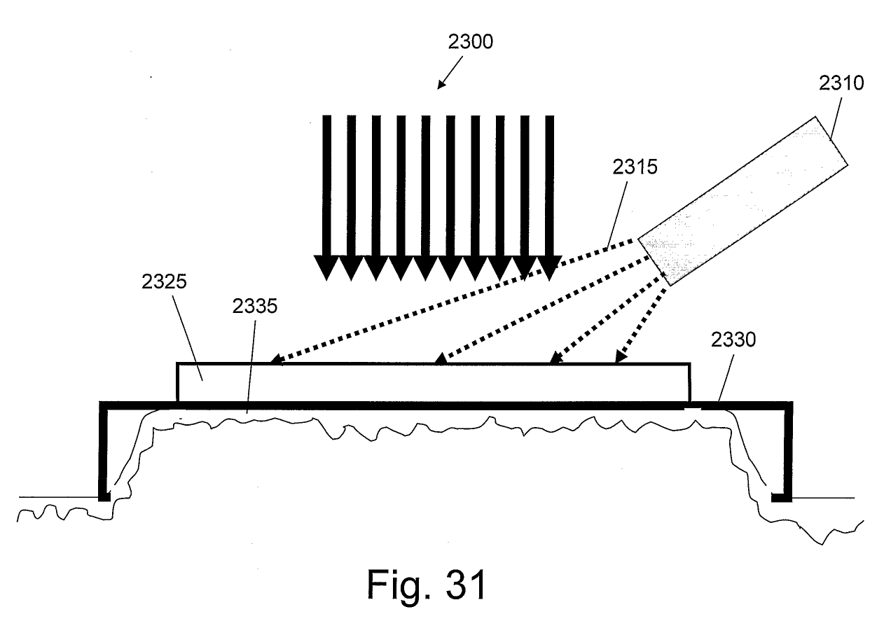

FIG. 30 illustrates an exemplary skin cooling device which is suitable for the pain alleviating apparatus of the present invention. Since the evacuation chamber is configured so as to ensure that a skin target contacts the transmitting element when a vacuum is applied, as described hereinabove, skin cooling is optimized when transmitting element 1906 is directly cooled. Accordingly, thermally conducting plate 1975, which is cooled by thermoelectric chiller 1979, or alternatively by means of a chilling liquid flowing over the conducting plate, contacts transmitting element 1906, in order to conduct the heat generated by the treated skin target 1960 from the transmitting element. The treatment handpiece is provided with chiller 1979 so as to prevent an increase in temperature of the epidermis, which may be damaged if the skin is relatively dark, e.g. Fitzpatrick skin type 4-6. In order to improve the compactness of the skin cooling device, plate 1975 is positioned obliquely with respect to transmitting element 1906 without interfering with the propagation of light beam 1908. It will be appreciated that pain alleviation is achieved by application of a vacuum, which brings the skin in contact with the transmitting element, and not by means of the chiller. As described in Example 8 hereinbelow, pain relief was noticeable during experimentation performed in conjunction with vacuum-assisted, light-based treatments without employment of a skin chiller.

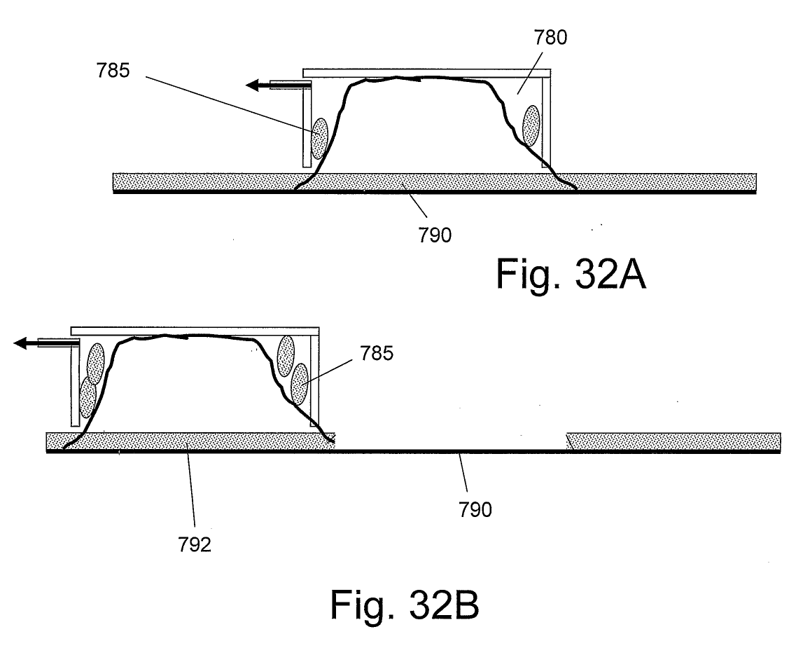

As shown in FIG. 31, the transmitting element may be alternatively cooled by applying a low temperature spray, such as produced by Dermachill, USA, to the transmitting element. Apparatus 2300 comprises pressurized can 2310, from which chilling vapors 2315 are sprayed onto transmitting element 2325 of evacuation chamber 2330, in order to chill transmitting element 2325 and underlying flattened skin target 2335. Such a chiller, which is provided with the Alexandrite laser produced by Candela Corporation, USA, chills the skin directly so that the epidermis achieves a very low temperature of less than 0° C. Due to the very low temperature of the epidermis, the effect of a chilling operation is noticeable for a period on the order of milliseconds rather than seconds, and therefore the chilling operation effectively protects the epidermis without chilling deeper skin regions. By selecting a transmitting element 2325 of a sufficiently thin thickness, the chiller is capable of chilling skin target 2335 as if transmitting element 2325 were not present. A transmitting element 2325 having a width of 150-500 microns and made from highly thermally conductive material such as sapphire is capable of chilling the skin with a spray which is regularly applied on uncovered skin. Epidermal chilling by the spray is made possible when the thermal relaxation time of a sapphire transmitting element is equal to, or less than, the thermal relaxation time of the epidermis, which is approximately 0.5 msec. Thin sapphire transmitting elements, e.g. having a thickness of 0.5 mm and a diameter of 1 inch may be obtained from Esco Products Inc., USA.

Apparatus for Preventing Gel-Caused Obstruction

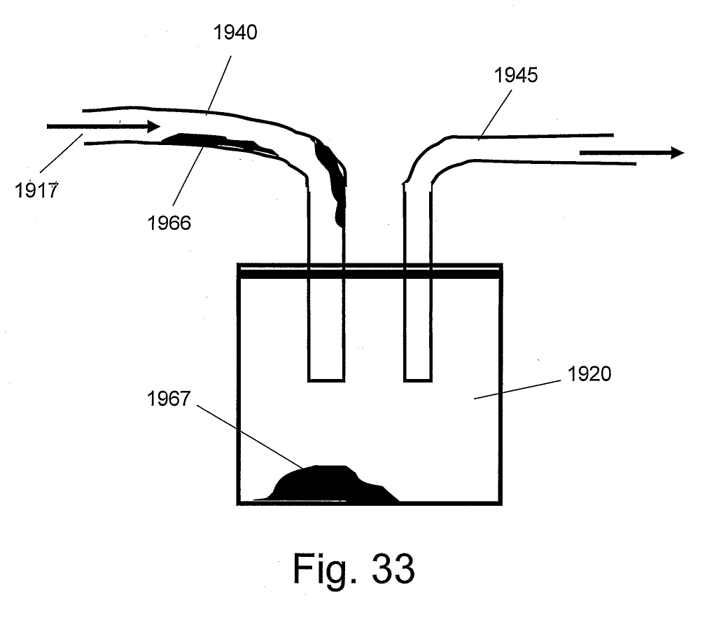

The apparatus may be advantageously provided with means to prevent the obstruction of the evacuation chamber conduits by heat releasing gel applied to the skin target prior to the treatment. As shown in FIGS. 32A and 32B, gel 785 is squeezed to the periphery of evacuation chamber 780 after application of a vacuum. When evacuation chamber 780 is displaced from skin area 790 to skin area 792, further gel is squeezed and accumulates, as shown in FIG. 32B. The gel is eventually aspirated into the evacuation chamber conduits, causing a significant risk of obstruction thereto when a large-diameter treatment beam normally associated with an IPL unit is used and necessitating the employment of a correspondingly large-diameter evacuation chamber. Without employing means to prevent passage of the gel, a large quantity of gel is liable to be drawn through the conduits and to the vacuum pump, eventually resulting in the malfunction of the latter and in less efficacious treatments. Also, aspirated gel tends to contaminate the evacuation chamber, and the cleaning or sterilization of the evacuation chamber prior to the treatment of another patient is difficult.

Referring back to FIG. 22, evacuation chamber 1901 has two passageways 1930 through which air is evacuated therefrom. Each passageway 1930, which is in fluid communication with the interior of evacuation chamber 1901, is defined by outer wall 1924, vertical portion 1926, and cylindrical horizontal wall 1930 connected to both outer wall 1924 and vertical portion 1926. The distal end of vertical portion 1926 is connected to transmitting element 1906, vertically spaced above, and interiorly spaced from, the distal end of outer wall 1924 placed on skin surface 1965, and is connected to vertical portion 1926 of passageway 1930. The top of horizontal passageway wall 1930 is vertically spaced above outer wall 1924, and evacuation chamber 1901 is therefore considered to be U-shaped. Each horizontal wall 1930 terminates with an opening 1917, which is separated from the distal end of outer wall 1924 by P and is laterally separated from centerline 1969 of evacuation chamber 1901 by J. While the gel may be drawn by the applied vacuum or may laterally slide from skin target 1960 after being pressed by transmitting element 1906, dimensions P and J are selected so as to ensure that the volume of the passageways 1930 and of the chamber interior between wall 1924 and the adjacent surface of drawn skin target 1960 is sufficiently large to prevent the obstruction of corresponding opening 1917 by gel 1963. For example, a evacuation chamber having a height K of 2 mm, a wall opening diameter of 3 mm, a separation P of 10 mm from the opening to the distal end of the wall, and a lateral separation J of 20 mm from the evacuation chamber centerline to the opening is sufficient to prevent obstruction of the opening by gel.

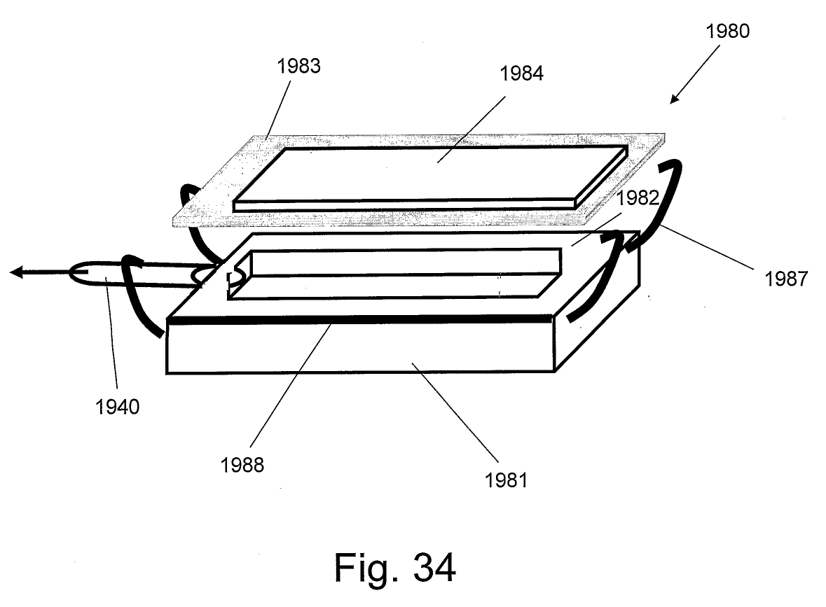

FIG. 33 illustrates another arrangement for preventing vacuum pump suction of gel. The arrangement includes trap 1920, conduit 1940 through which gel and air are drawn from the evacuation chamber to trap 1920, and conduit 1945 through which air is drawn from trap 1920 to the vacuum pump, all of which may be disposable. Air evacuated from the evacuation chamber through opening 1917 flow through conduits 1940 and 1945 until introduced to the inlet port of the vacuum pump. The gel which is evacuated from the evacuation chamber collects within trap 1920. Trap 1920 is periodically emptied so that the accumulated gel does not rise above the inlet of conduit 1945. Trap 1920 and conduits 1940 and 1945 are preferably made from a plastic hydrophilic material, to urge the gel to cling to the walls thereof rather than to be drawn through the conduits to the vacuum pump. As shown, gel 1966 clings to the walls of conduit 1940 and gel 1967 is collected on the bottom of trap 1920. The conduits may be suitably sized to prevent the passage of gel to the vacuum pump. For example, the diameter of conduit 1940 at the vacuum wall opening is 30 mm and narrows to a diameter of 10 mm at the discharge to trap 1920, and the diameter of conduit 1945 at the inlet side is 5 mm and is 10 mm at the discharge side in the vicinity of the the vacuum pump inlet port.

Other arrangements for preventing vacuum pump suction of gel may also be employed. For example, the gel may be bound to a suitable ion exchange resin introduced into trap 1920 and thereby be prevented from being drawn through conduit 1945. If so desired, a filter may be provided at the inlet of conduits 1940 and 1945.

Alternatively, gel may be prevented from exiting the evacuation chamber by increasing the diameter of conduit 1940 at the vacuum wall opening. Consequently, the inwardly directed force acting on the gel which has laterally slid from a drawn skin target by means of the atmospheric air introduced to the evacuation chamber via conduit 1940 during a vacuum release mode is sufficient to prevent the gel from exiting the evacuation chamber. A hydrophobic coating, such as silicon or teflon, may be applied onto the evacuation chamber walls, so that the gel will be prevented from adhering to the evacuation chamber walls, particularly during a vacuum release mode. Instead of adhering to the evacuation chamber walls, the gel falls to the skin surface. Advantageously, gel is therefore not transported to another skin target during the repositioning of the handpiece, but rather assumes the shape of the distal end of the evacuation chamber walls. If the distal end of the evacuation chamber walls is circular, for example, the gel that falls to the skin surface during a vacuum release mode is also circular, indicating to the health professional that is supervising the treatment that the given skin surface has already been impinged by the treatment light.

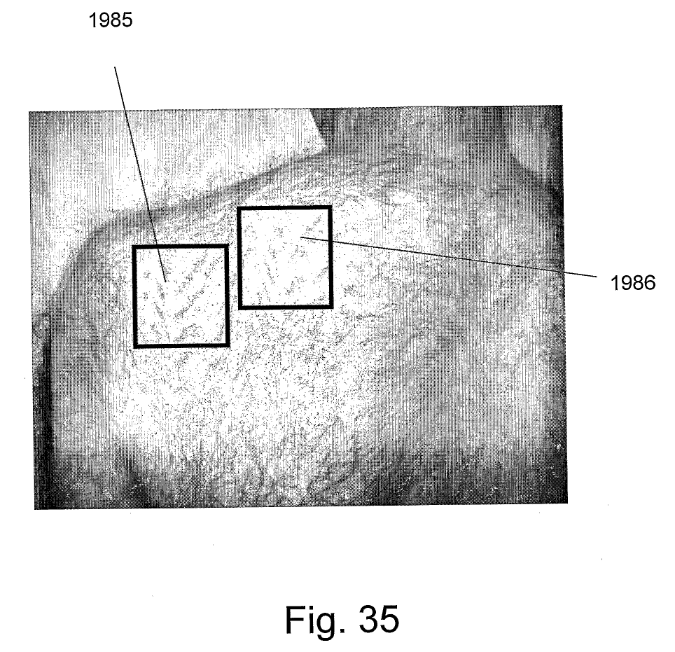

In FIG. 34, apparatus 1980 comprises an evacuation chamber having a detachable upper portion, so that the gel retained by the evacuation chamber interior walls may be removed therefrom, such as by dissolving the gel with salt or with any other suitable dissolving agent. Apparatus 1980 comprises upper portion 1983 having an open central area, transmitting element 1984 attached to upper portion 1983, evacuation chamber walls 1981, evacuation chamber cover 1982 perpendicular to walls 1981 and suitably sized so as to support upper portion 1983, and a plurality of attachment clips 1987 pivotally connected to a corresponding evacuation chamber wall 1981 for detachably securing upper portion 1983 to evacuation chamber cover 1982. Thin compliant sealing element 1988 is preferably attached to the periphery of evacuation chamber cover 1982, to prevent infiltration of atmospheric air into the evacuation chamber. Conduit 1940 is shown to be in communication with the interior of the evacuation chamber.

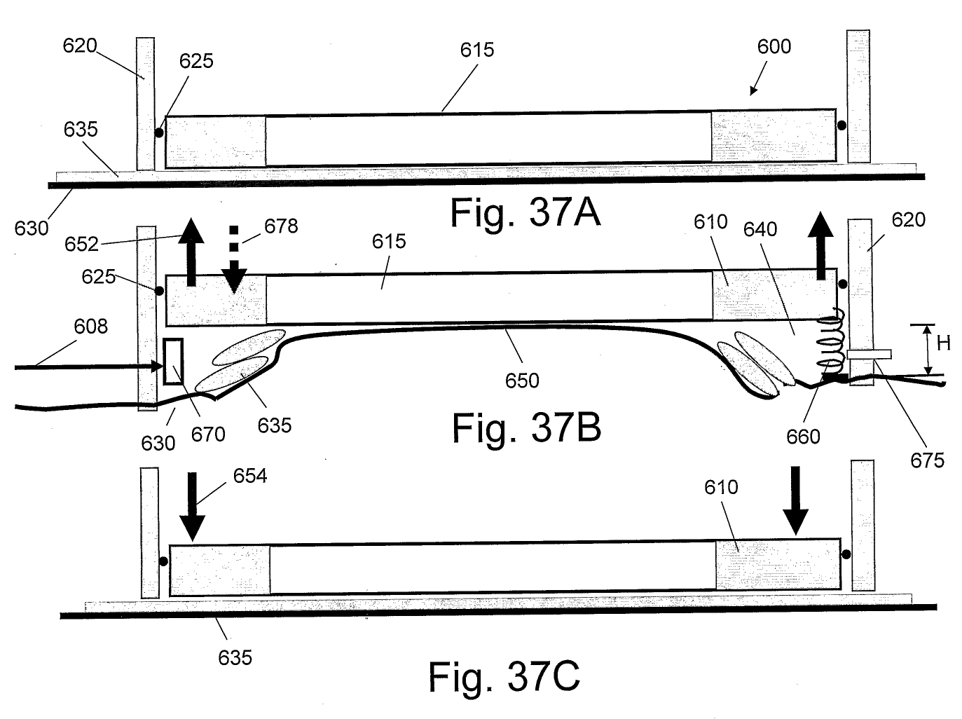

FIG. 36 illustrates another embodiment of apparatus for preventing the obstruction of evacuation chamber conduits by heat releasing gel during vacuum-assisted light-based treatments of the skin. Apparatus 400 comprises evacuation chamber 420, peristaltic pump 430, vacuum controller 440, control valve 450, and micro-switch 460.

The vacuum applying mode is initiated upon transmission of signal 445 to controller 440, following which peristaltic pump 430 is activated. Peristaltic pump 430 comprises hose 442 connected to conduit 425 in communication with the interior of evacuation chamber 420 and rotatable hub 446, from which a plurality of shoes and/or rollers 448 (referred to hereinafter as “pressing elements”) radially extend. As hub 446 rotates, the pressing elements sequentially squeeze a different region of hose 442 and a volume of fluid entrapped by two adjacent pressing elements is thereby forced to flow unidirectionally through hose 442 by a positive displacement action towards end 449 thereof. Consequently, when peristaltic pump 430 is activated, air is drawn from the interior of evacuation chamber 420 to generate a vacuum therein ranging from 0-1 atmospheres. If a considerable amount of gel 405 accumulates within the periphery of evacuation chamber 420, the gel is also forced to flow within hose 442 without causing any obstruction to the latter. The gel that is discharged from end 449 of hose 442 falls onto skin surface 410, indicating that an adjoining skin target 415 has undergone a light-based treatment.

Micro-switch 460, or any other suitable skin contact detector, is adapted to sense the placement of the handpiece or of evacuation chamber chamber 420, onto skin target 415. Micro-switch 460 generates signal 445 upon sensing the placement of evacuation chamber 420 on skin target 415. Control valve 450 is triggered by a light detector (not shown), which generates signal 455 upon detecting the termination of the light-based treatment pulse 470. Control valve 450 is opened after the generation of signal 455, to introduce atmospheric pressure air 452 to the interior of evacuation chamber 420 via passageway 456 and to thereby initiate the vacuum release mode. Signal 455 is also transmitted to controller 440, to deactivate peristaltic pump 430. The described automatic operation of peristaltic pump 430 therefore prevents the patient from suffering pain during the associated treatment. If so desired, the operation of peristaltic pump 430 may be manually overridden.

It will be appreciated that a peristaltic pump or a contact detector may be employed in conjunction with any other embodiment of the invention.

In another embodiment, a vacuum pump that is suitable for drawing both air and gel is configured in similar fashion to a Wankel mechanism well known in the field of combustion engines, in which a triangular rotor rotates on an eccentric shaft inside an epitrochoidal casing. The pump comprises only 3-5 parts, resulting in simple and low cost production. The pump has a low power consumption ranging from 1-10 W, e.g. approximately 5 W, so that it may be powered by an inexpensive battery, e.g. a rechargeable battery, housed within the treatment handpiece. The extremely low power consumption of the pump is made possible by virtue of the following factors: a) The pump, including its casing, rotor and covers, is made from a self-lubricating material, such as Acetal mixed with Teflon, e.g. having a friction coefficient of 0.05, which minimizes friction and therefore similarly reduces the power consumption. b) A thin layer of gel which is drawn by, and transferred within the various compartments of, the pump adds to the pump lubrication. c) The pump rotor is formed with slots, so that the rotor may conform to the shape of the casing and flex in response to gel pressurization, thereby reducing resistance to the rotation of the rotor.

Although the gel may provide lubrication for the pump when drawn from the evacuation chamber to the pump cavity, it is desirable that the pump be made from a self-lubricating material to prevent overheating or malfunction thereof since the skin may be covered with a very thin layer of gel, or may not be covered at all by gel, and therefore the pump may not be adequately lubricated.

In another embodiment, the vacuum pump is an air pump. When air is evacuated from the evacuation chamber, a piston (not shown) which is normally closed by a spring is opened to allow air to be aspirated. During the vacuum release mode, the piston is set to its original position, returning air to the evacuation chamber and any aspirated gel to the skin surface.

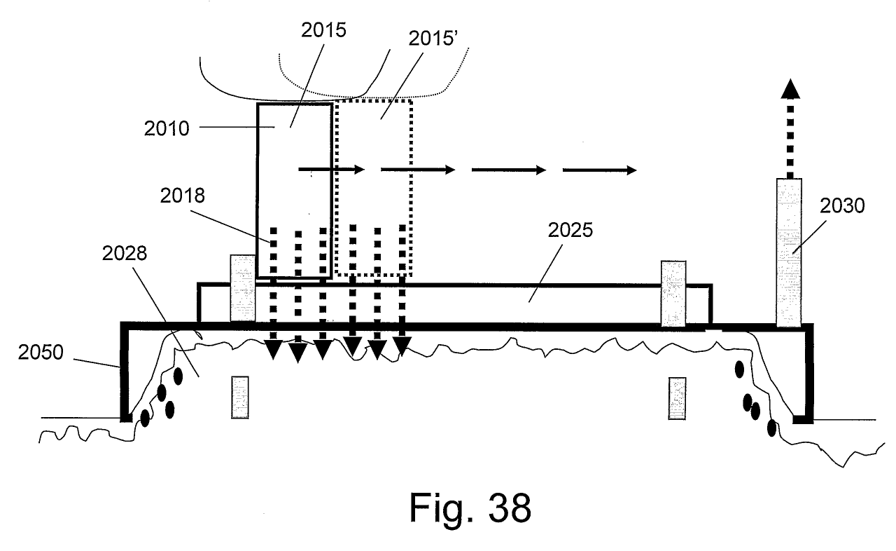

FIGS. 37A-C illustrate another embodiment of the invention wherein a vacuum pump is not needed for vacuum-assisted light-based treatments of the skin. Apparatus 600 comprises a vertically displaceable cover 610 to which transmitting element 615 is secured, chamber walls 620 in which vertically displaceable cover 610 is mounted, and sealing element 625 which is secured to the outer periphery of cover 610. Chamber walls 620 surround, and are of a similar shape as, cover 610.

When cover 610 is in its lowermost position, as shown in FIG. 37A, the cover is flush with skin surface 630 on which is applied a layer of gel 635. In this position, air is prevented from infiltrating between cover 610 and skin target 630, e.g. by means of a sealing element externally affixed to walls 620. When a proximally directed force represented by arrows 652 is applied to cover 610, as shown in FIG. 37B, the cover is raised while sealing element 625 resiliently contacts walls 620. Apparatus 600 is configured such that distal displacement of cover 610 is prevented after having been raised, without application of a subsequent distally directed force. While cover 610 is raised, an evacuation chamber 640 is produced internally to chamber walls 620, due to the increased volume between cover 610 and skin surface 630 while air is prevented from infiltrating therein. The vacuum generated within evacuation chamber 640 as a result of the proximal displacement of cover 610 ranges from 0-1 atmospheres and is suitable for drawing skin target 650 towards the displaced cover 610 as shown, in order to be subsequently impinged by a treatment pulse. When a distally directed force represented by arrows 654 is applied to cover 610 following the light-based treatment, as shown in FIG. 37C, cover 610 returns to its lowermost position in preparation for displacement to the next skin target. Aeration tube 675 in communication with a manually operated or control valve (not shown) may be employed to quicken distal displacement of cover 610 during a vacuum release mode by introducing atmospheric air to evacuation chamber 640 upon conclusion of the skin target treatment.

Proximally directed force 652 or distally directed force 654 may be generated manually by means of handles (not shown) attached to cover 610, or electrically by means of a plurality of solenoids 670 and/or by means of a spring assembly 660 deployed around the periphery of cover 610, as well known to those skilled in the art to achieve balanced displacement of the cover. Solenoids 670 are mounted such that one side of a solenoid is mechanically connected to displaceable cover 610 and the other side thereof is connected to a chamber wall 620. When electrical actuation of cover 610 is employed, command 608 generated by skin contact sensor 460 (FIG. 36) is transmitted to spring assembly 660 or solenoids 670 after a predetermined time delay following contact between cover 610 and skin surface 630, causing cover 610 to be proximally displaced upward with a proximally directed lifting force 652 comparable to that of a piston. By properly controlling solenoids 670, height H of the drawn skin target 650 relative to the adjoining skin surface 630 can be adjusted. Height H of the drawn skin is generally increased as the treatment spot is increased. For example, height H may be 2 mm for a treatment spot of 40 mm, while height H may be 0.5 mm for a treatment spot of 3 mm. Alternatively, height H may be adjusted to ensure that skin target 650 contacts transmitting element 615 for pain alleviation.

At times, a sufficiently high vacuum level for effecting a light-based treatment may not be produced within evacuation chamber 640, due to a malfunction. If a health professional notices that the distance between skin target 650 and transmitting element 615 is greater than a predetermined distance for effective treatment with an IPL or laser, the automatic control of cover 610 may be overridden. By reversing the direction of current within solenoids 670, one-time distally directed force 678 may be generated which urges cover 610 towards skin surface 630.

When the distal end of the treatment light source is positioned on chamber walls 620, cover 610 has a relatively low weight of approximately 50 gm. However, if the treatment handpiece is positioned on cover 610 such that the combined weight of the cover and handpiece is approximately 1 kg, the capacity of solenoids 670 needs to be increased, in order to raise both the cover and handpiece and to produce a vacuum within chamber 640.

Apparatus 600 advantageously provides low power consumption and increased compactness. When the handpiece is positioned on chamber walls 620, solenoids 670 are energized by a battery without need of draining wall current and only when cover 610 is needed to be vertically displaced. The energy requirement for raising cover 610 to a height of 2 mm is approximately 0.5 J for a typical 500-pulse large area treatment on the back or legs. Therefore an inexpensive 1.5 V battery is suitable for more than 1000 treatments.

Apparatus 600 also advantageously prevents accumulation of gel. When skin target 650 is drawn during a vacuum applying mode as shown in FIG. 37B, gel 635 is displaced to a peripheral skin area within evacuation chamber 640. However, when cover 610 returns to its original lowermost position as shown in FIG. 37C, skin target 650 is retracted. Gel 635 is then substantially uniformly spread underneath cover 610, due to the pressure applied by cover 610. Similarly when apparatus 600 is repositioned to another skin target, gel 635 does not accumulate.

The proximally directed force may be supplemented by means of a vacuum pump, which may be needed if an excessive amount of gel is applied to skin surface 630 or if it desired to indicate that skin target 650 has undergone a light-based treatment as described hereinabove.

Skin Gliding Apparatus

Some light-based hair removal devices operate at high repetition rates which enable fast treatment by gliding the device over the skin. An example of such a device is the Light Sheer diode laser manufactured by Lumenis which can operate at a repetition rate of 2 pulses per sec. The size of the laser exit beam is approximately 10×10 mm. The laser is highly efficient at 40 J/cm²; however, it is very painful, attaining a pain level of 5.

When the medical treatment is administered solely by means of a laser, the evacuation chamber may be provided with skin gliding apparatus. Very fast and painless treatments may be performed by gliding the laser unit distal end at a speed ranging from 0.3-40 cm/sec over an interface element made of sapphire through which the laser light can be transmitted. A gliding action is made possible by means of a suitable track formed in, or attached to, the interface element. The track supports the laser unit distal end, and is adapted to minimize friction between the laser distal end and the interface element, and to prevent the latter from being scratched. The skin gliding apparatus is preferably configured in such a way so as to maintain the laser unit distal end in a disposition which is substantially perpendicular with respect to the interface element and to prevent overlaps or voids between adjacent spots that are treated by the treatment light. Pain is absent due to the relatively large size of the interface element, which ensures that a sufficiently large number of pressure receptors are squeezed so that a signal transmitted therefrom inhibits reception of a pain signal, and due to the relatively high vacuum level. In contrast to prior art treatments wherein immediate sharp pain is felt during each treatment pulse, necessitating a patient to rest during a long delay before continuing the treatment or to be applied with a risky analgesic topical cream, the treatment speed of apparatus of the present invention employing an evacuation chamber need not be slowed.

For example, an evacuation chamber having a size of e.g. 20×40 mm is suitable for inhibiting pain in conjunction with treatment light generated by the Light Sheer diode laser having an energy density of 40 J/cm². The laser unit distal end may be displaced over a sapphire interface element at a speed of 10 mm every 0.5 seconds. The applied vacuum is maintained for a duration of 4 seconds, thereby allowing a skin surface having a similar area of 20×40 mm to be treated by the treatment light without having to release the vacuum.

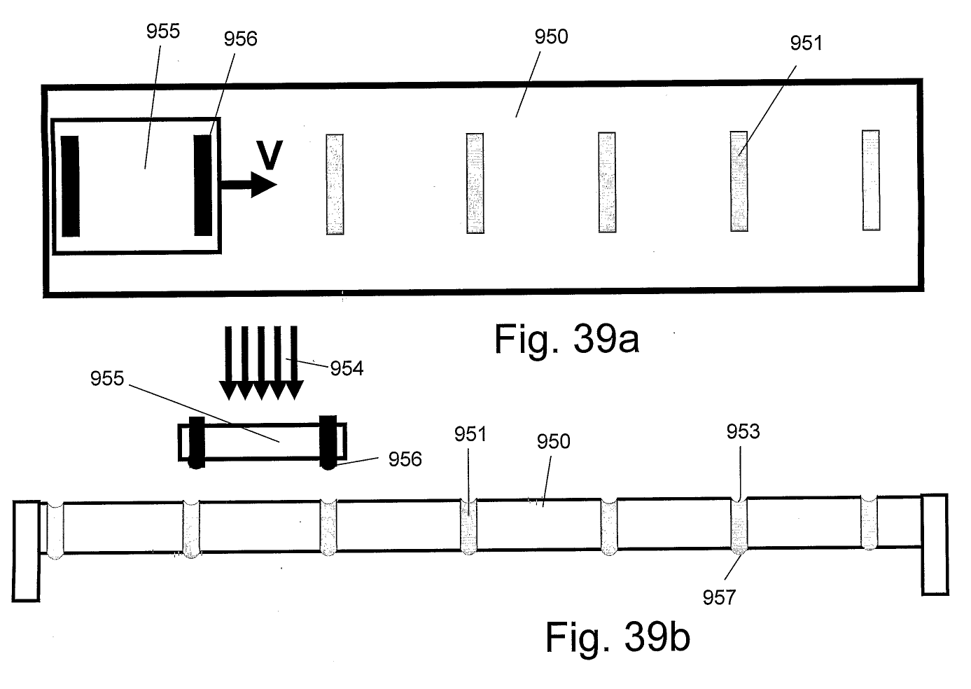

FIG. 38 schematically illustrates the gliding of a laser distal end on the transmitting element of an evacuation chamber with respect to the following steps:

A) Laser distal end 2010 is initially positioned in contact with the top of transmitting element 2025 of evacuation chamber 2050 at position 2015.

B) Air is evacuated from evacuation chamber 2050 via conduit 2030 within 0.5 sec at a vacuum level of at least 500 mmHg which is suitable for inducing pain inhibition.

C) Treatment laser pulse 2018 is fired at position 2015 towards skin target 2028 therebelow.

D) Laser distal end 2010 is displaced to position 2015′ at a speed of L/t, where L is the beam diameter and t is the interval between laser pulses. The laser distal end may be automatically and cyclically repositioned if the gliding track is provided with equally spaced stations, whereat the laser distal end is urged to be stationary when light is emitted therefrom.

E) Treatment laser pulse 2018 is fired at position 2015′ towards the skin target therebelow.

F) Steps D) and E) are repeated until laser distal end 2010 is displaced along the entire surface area of transmitting element 2025.

G) Laser distal end 2010 is displaced to original position 2015.

H) The vacuum within evacuation chamber 2050 is released within 0.5 second.

I) Evacuation chamber 2050 is raised and repositioned.

The displacement of laser distal end 2010 may be externally triggered, i.e. by means of an optical detector that senses the presence of a marker on transmitting element 2025 that corresponds to each target position. Alternatively, laser distal end 2010 is driven by a suitable mechanism at a constant speed of L/t over transmitting element 2025 in free running fashion, i.e. not externally triggered. For example, a laser distal end that produces a 12-mm diameter light beam, such as the Light Sheer of Lumenis, will be driven at a speed of 20 mm/sec if the laser is operated in a free running mode at a 2 Hz repetition rate. In the free running mode, a photodiode may be employed, which is adapted to detect a light pulse generated by the laser and to generate an audible signal being indicative that the laser distal end may be repositioned.

In another embodiment, gliding laser distal end 2010 is fired in response to a texture sensing mechanism. A texture sensing mechanism is operable in conjunction with a laser such as the Fraxel® laser produced by Reliant Technologies Inc., USA, which is suitable for skin rejuvenation and known to be very painful. The Fraxel® laser is generally activated upon detecting the presence of a blue dye which is applied to the skin and helps to identify a skin target desired to be treated by laser beam 268. In this embodiment of the present invention, a texture associated with transmitting element 2025, such as a blue dye or a poorly polished portion of the transmitting element, may activate the Fraxel-type laser when the presence of the texture is sensed. When a vacuum is applied to evacuation chamber 2050 and skin target 2028 is compressed and flattened against transmitting element 2025, the skin target is disposed in relatively close proximity to distal end 2010 of the laser. The laser may function in similar fashion as the Fraxel® laser; however, if a sufficiently high vacuum level is applied to evacuation chamber 2050, the selected medical treatment will be painless.

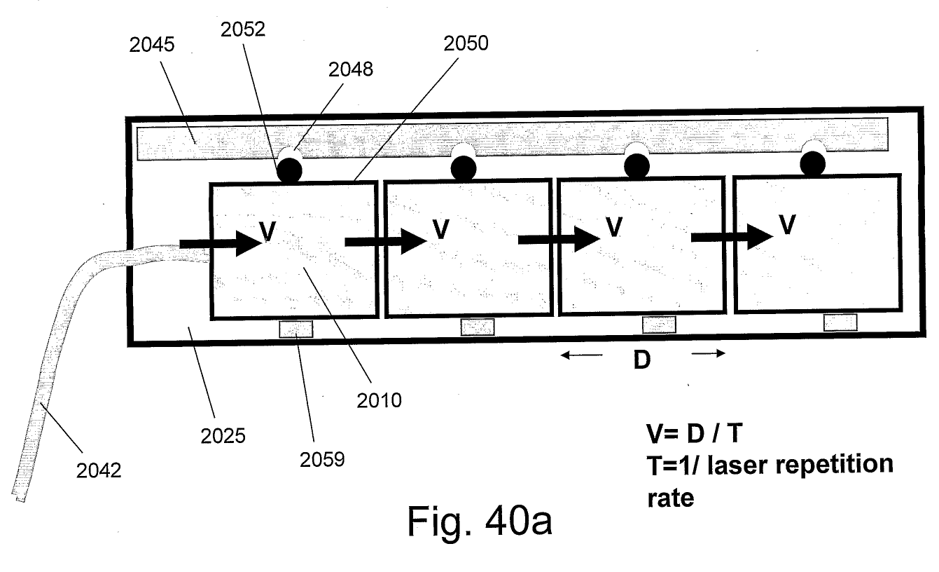

FIGS. 39a and 39b illustrate top and side views, respectively, of a transmitting element of an evacuation chamber which is provided with another configuration of bipolar RF-assisted metallic conducting electrodes suitable for skin flattening and pain inhibition in conjunction with laser or IPL treatment light. Sapphire transmitting element 950 is formed with a plurality of slits which are filled with a metallic material such as aluminum, to produce electrodes 951. The dimensions of the slits may be for example a length of 17 mm, a width of 2 mm, and a spacing between two adjacent slits of 30 mm. Electrodes 951 are formed such that the uppermost portion 953 thereof is concave and the lowermost portion 957 thereof in contact with drawn, flattened skin is convex. The concave shape of uppermost portion 953 facilitates the seating therein of RF electrodes 956 provided at the distal end of an IPL or laser unit 955, such as one manufactured by Syneron Medical Ltd., Israel, which generates light 954 transmitted through transmitting element 950. The convex shape of lowermost portion 957 provides good contact with the skin.

By employing such a configuration of electrodes 951, the RF-assisted IPL or laser unit 955 can be glided upon transmitting element 950 at a high speed of V, e.g. capable of moving a distance of 30 mm within 10 millisec. Convex electrodes 956 of IPL or laser unit 955 will therefore be quickly seated into the corresponding concave portions 953 of electrodes 951 above a selected skin target prior to be treated by light 954.

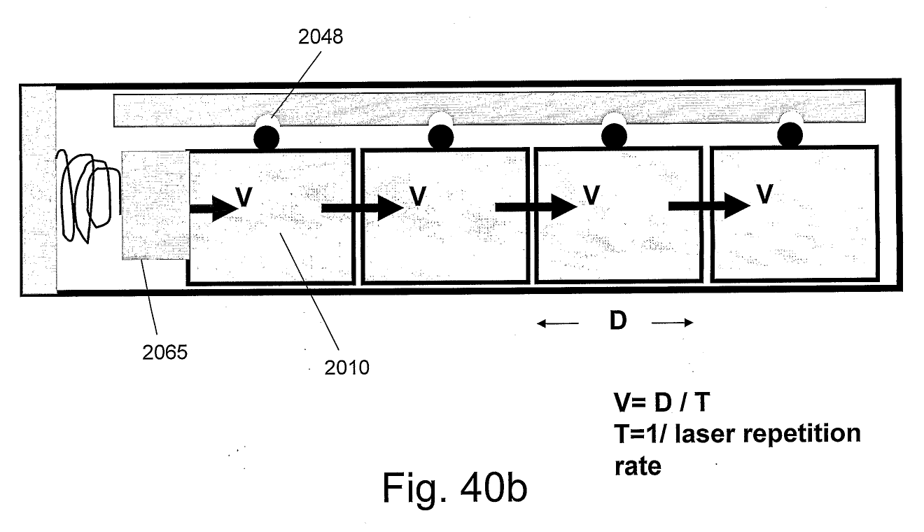

FIGS. 40a and 40b schematically illustrate two driving means, respectively, for gliding a laser distal end 2010 having a size D over the top surface of transmitting element 2025 of a pain inhibiting evacuation chamber.

In FIG. 40a, the driving means is a pneumatic tube 2042 which displaces laser distal end 2010 at a constant speed, or is manual force. A linear ruler 2045 for measuring the displacement of distal end 2025, in which equally spaced apertures 2048 are bored, is attached to transmitting element 2025. Laser distal end 2010 has a frame 2050, to which a spring biased spherical element 2052 for enabling laser distal end 2010 to be linearly displaced along the ruler is attached. Spring 2059 urges spherical element 2052 into a corresponding aperture 2048 whenever a spherical element 2052 is in front of the corresponding aperture 2048. By quickly driving laser distal end 2010, the latter is displaced from aperture to aperture by discrete steps, so that a treatment pulse may be fired at each subsequent step. If laser distal end 2010 is displaced by manual force, the force for disengaging spherical element 2052 from the aperture 2048 in which it is seated can be controlled by selecting the strength of spring 2059. Spring strength is selected to enable disengagement of spherical element 2052 from a corresponding aperture 2048 within a time duration T inversely proportional to the laser repetition rate. As a result, laser distal end 2010 is synchronously displaced with respect to the free running laser repetition rate at a speed of V which is equal to D/T, so that the skin surface under the evacuation chamber may be uniformly treated. A photodiode (not shown) may be employed to detect a laser or IPL pulse and to generate an audible signal, thereby enabling the synchronization of the laser distal end displacement with the laser operation.

In FIG. 40b, the driving means is spring motor 2065, which is provided with a suitable transmission or actuator to linearly displace laser distal end 2010 from one aperture 2048 to another.

Apparatus for Controlling Depth of Light Absorption

In another embodiment, the apparatus of the invention is adapted to increase the concentration of blood vessels in the vicinity of the skin target. The added concentration of blood vessels increases the absorption of light within the tissue, and therefore facilitates treatment of a skin disorder.

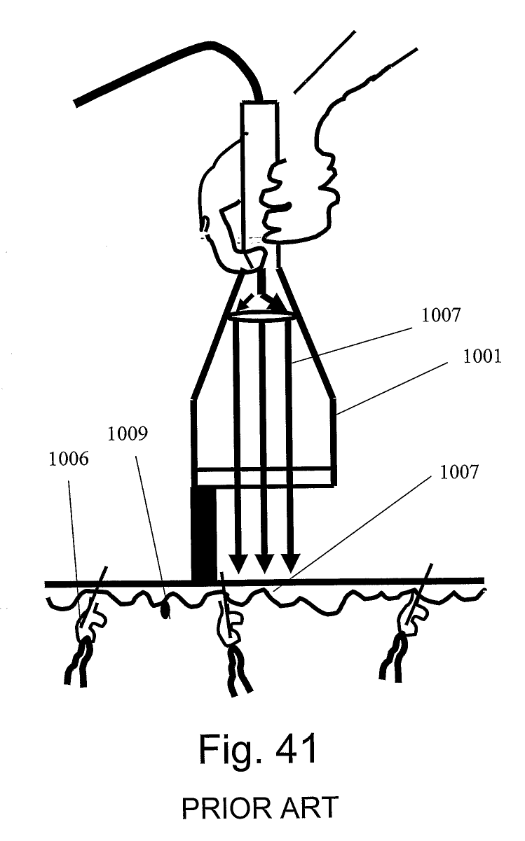

FIG. 41 illustrates the propagation of an intense pulsed laser beam the wavelength of which is in the visible or near infrared region of the spectrum, i.e. shorter than 1800 nm, from the distal end of a handpiece to a skin target according to a prior art method. Handpiece 1001 comprises transmitting element 1002, such as a lens or a window, which transmits monochromatic beam 1007 emitted from the laser unit and impinges skin target 1004. The beam penetrates skin target 1004 and selectively impinges a subcutaneous skin structure to be thermally injured, such as collagen bundle 1005, blood vessel 1009, or hair follicle 1006. In this method, external pressure or vacuum is not applied to the skin.

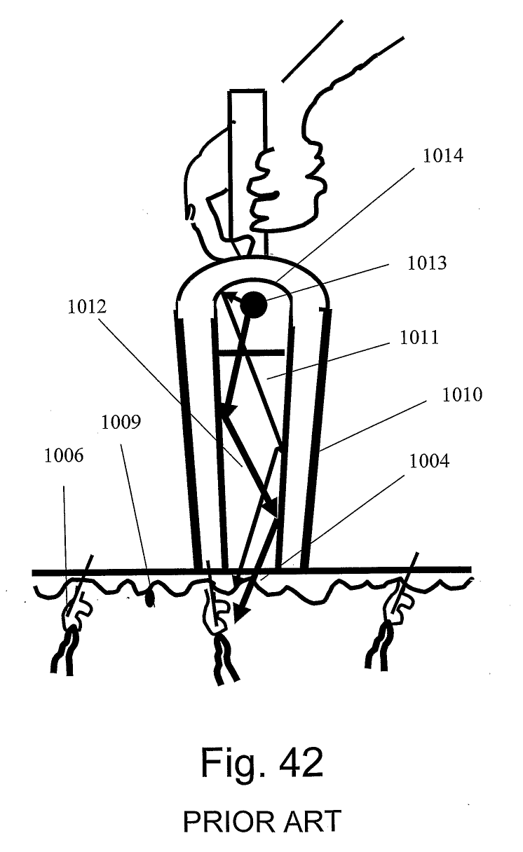

FIG. 42 illustrates a prior art non-coherent intense pulsed light system from which light is fired to a skin target for e.g. treatment of vascular lesions, hair removal, or photorejuvenation. Handpiece 1010 comprises light guide 1011 which is in contact with skin target 1004. Beam 1012, which is generated by lamp 1013 and reflected from reflector 1014, is non-coherent and further reflected by the light guide walls. In some handpieces, such as those produced by Deka (Italy), a transmitting element is utilized, rather than a light guide. Chilling gel is often applied to the skin when such a light system is employed. In this method, external pressure or vacuum is not applied to the skin, and the handpiece is gently placed on the skin target, so as to avoid removal of the gel layer, the thickness of which is desired to remain at approximately 0.5 mm.

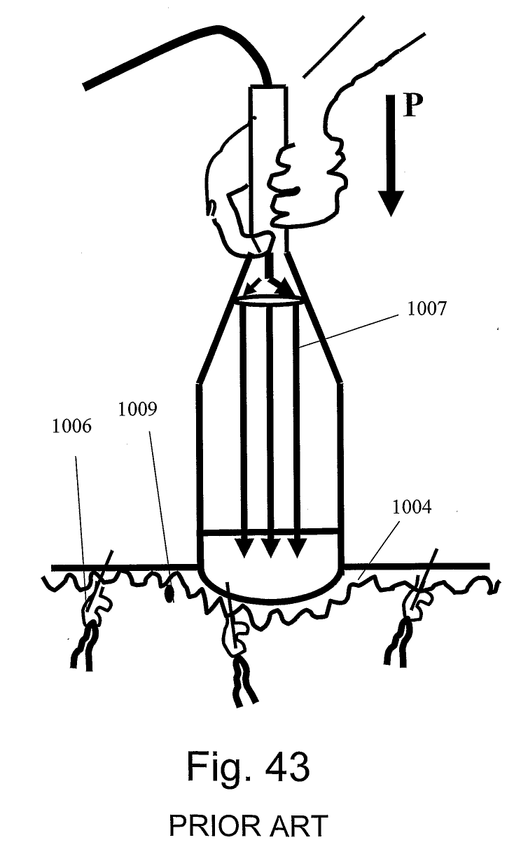

FIG. 43 illustrates a prior art laser system similar to those of U.S. Pat. Nos. 5,595,568 and 5,735,844, which employs an optical component 1022 at the distal end thereof in contact with skin target 1004. Pressure is applied to skin target 1004, in order to expel blood from those portions of blood vessels 1025, as schematically illustrated by the arrows, which are in the optical path of subcutaneously scattered light, thereby allowing more monochromatic light to impinge hair follicle 1006 or collagen bundle 1005. Concerning hair removal, melanin is generally utilized as an absorbing chromophore.

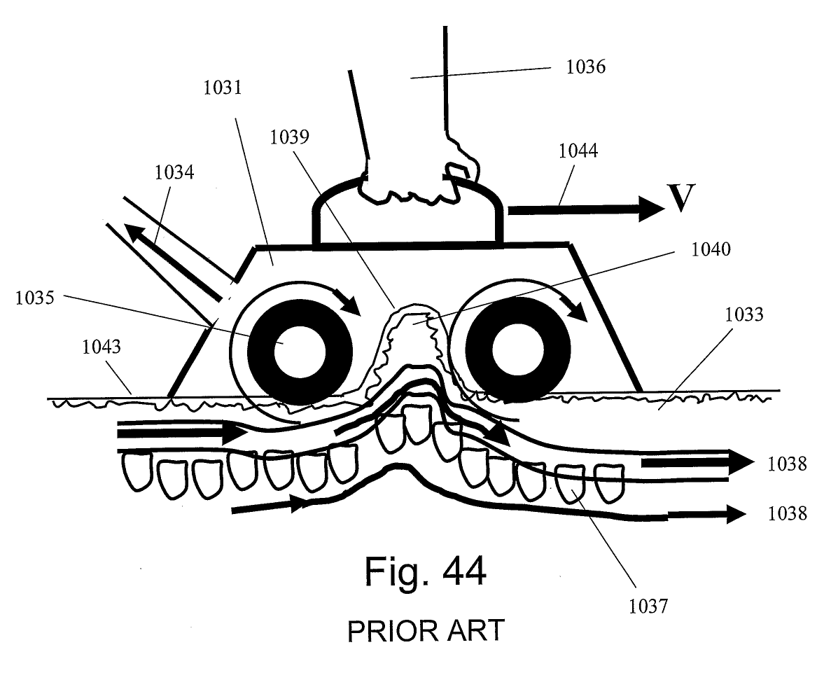

FIG. 44 illustrates a prior art device 1031, such as that produced by LPG (France), which is in pressing contact with skin 1033 in order to perform a deep massage of cellulite adipose layer 1037. Device 1031 is formed with a convex surface 1039 in a central region of its planar skin contacting surface 1043. Device 1031 stimulates the flow of lymphatic fluids in their natural flow direction 1038 in order to remove toxic materials from the adjoining tissue. The stimulation of lymphatic fluid flow is achieved by applying a vaccum to the interior of device 1031 so that air is sucked therefrom in the direction of arrow 1034 of the skin. The application of the vacuum draws skin toward convex surface 1039 and induces the temporary formation of skin fold 1040, which is raised in respect to adjoining skin 1033. Due to the elasticity of skin, skin fold 1040 returns to its original configuration, similar to the adjoining skin, upon subsequent movement of device 1031, while another skin fold is formed. As device 1031 is moved by hand 1036 of a masseur in direction 1044 of the device, similar to natural flow direction 1038, the lymphatic fluids flow in their natural flow direction. However, the lymphatic fluids will not flow if device 1031 were moved in a direction opposite to direction 1044. Wheels 1035 enable constant movement of device 1031.

In some cellulite massage devices, such as those produced by Deka (Italy) or the Lumicell Touch (USA), a low power continuous working infrared light source with a power level of 0.1-2 W/cm² provides deep heating of the cellulite area and additional stimulation of lymphatic flow. Such a light source is incapable of varying the temperature by more than 2-3° C., since higher temperatures would be injurious to the tissue and cause hyperthermia. Consequently these massage devices are unable to attain the temperatures necessary for achieving selective thermal injury of blood vessels, hair follicles or for the smoothening of fine wrinkles. Due to the movement of the device, the amount of optical energy, e.g. by means of an optical meter, to be applied to the skin cannot be accurately determined.

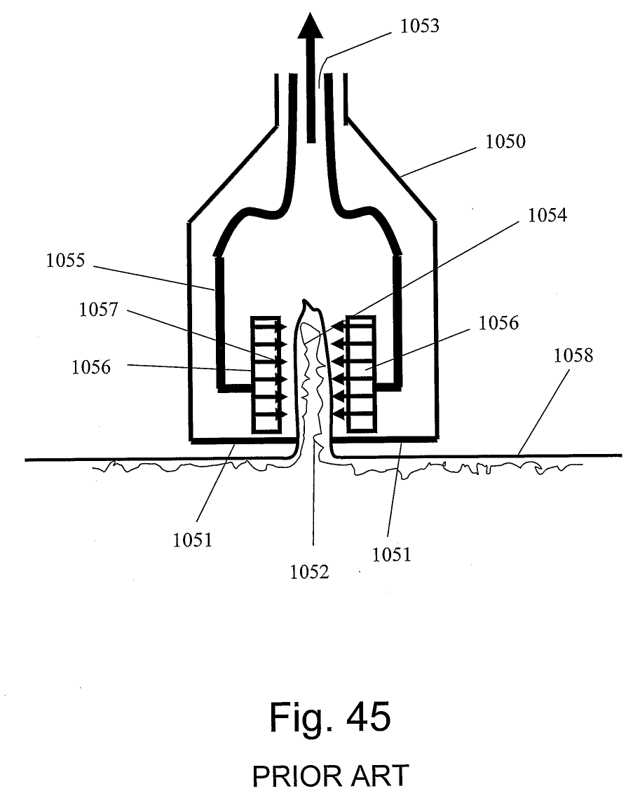

FIG. 45 illustrates a prior art hair removal device, similar to the device of U.S. Pat. No. 5,735,844, which is provided with a slot 1052 within a central region of skin contacting surface 1051 of handpiece 1050. When handpiece 1050 is placed on skin surface 1058 and a vacuum is applied to the handpiece via opening 1053, skin fold 1054 is formed. A narrow slot 1052 induces formation of a correspondingly longer skin fold 1054. Optical radiation is transmitted to the two opposed sides 1056 of skin fold 1054 by a corresponding optical fiber 1055 and optical element 1057. Upon application of the vacuum, skin fold 1054 is squeezed to prevent blood flow therethrough. This device is therefore intended to reduce the concentration of blood within skin fold 1054, in order to increase illumination of melanin-rich hair shafts, in contrast with the apparatus of one embodiment of this invention by which blood concentration is increased within the slight vacuum-induced skin protrusion so as to induce increased light absorption, as will be described hereinafter. Furthermore, this prior art device, due to the reduced concentration of blood within skin fold 1054, is not suitable for treatment of vascular lesions, photorejuvination, or the method of hair removal which is aided by the absorption of optical energy by blood vessels that surround or underlay hair follicles (as opposed to the method of hair removal which is aided by the absorption of optical energy by melanin).

Although the application of a vacuum to a skin surface has been employed in the prior art to supplement skin treatments performed by means of optical energy, many significant differences between prior art apparatus for a vacuum-assisted light-based skin treatment to that of the present invention are evident:

a) The prior art application of vacuum is intended to remove smoke or vapors caused by the light-based ablation of a skin surface. By the apparatus of the present invention, in contrast, the optical energy does not interact with the skin surface, but rather is targeted to subcutaneous skin structures without producing smoke or vapors.

b) In order to remove smoke and vapors produced by a prior art light-based skin treatment, a flushing process is required whereby the produced smoke and vapors are purged and replaced by clean air. A low vacuum level is therefore generated, since if a high level vacuum were generated, the treatment handpiece would be prevented from being lifted and displaced from one skin target to another. In contrast, a high vacuum level of approximately 0 atmoshpheres is generated in the method of the present invention to sufficiently draw the skin into the vaccum chamber and to therefore facilitate the treatment of a skin disorder, yet the treatment handpiece may be quickly repositioned from one skin target to another.

c) Since smoke or vapor removal by means of prior art apparatus prevents the same from adhering to the distal window of a light source, the vacuum application by prior art apparatus should immediately follow each light treatment pulse. The apparatus of one embodiment of the present invention, in contrast, stimulates an increase in blood vessel concentration by applying the vacuum in order to increase light absorption, and therefore the vacuum needs to be applied prior to the firing of the treatment beam.

d) Prior art apparatus does not provide means to temporarily modulate the vacuum level. In contrast, the apparatus of the present invention has control means for modulating the applied vacuum level, by which the optical absorptivity of a skin target may be adjusted in order to effect a desired treatment.

e) Evacuation of skin ablation and of smoke or debris by means of prior art apparatus precludes employment of a protective gel layer over the skin, since the gel forms a barrier between the skin surface and the ambient air. Even if a prior art apparatus were conducive to the application of gel, no provision is made to prevent obstruction of the vacuum pump. In contrast, the apparatus of the present invention allows for the application of gel to the skin prior to a vacuum-assisted non-ablative treatment, since the light-based treatment is subcutaneous, and furthermore, provides means for preventing the obstruction of the vacuum pump.

f) With respect to apparatus of the prior art which is intended to induce blood expulsion from local skin tissue, the treatment beam is limited, to a laser beam of approximately 5 mm. If the treatment beam were significantly larger, e.g. 40 mm, blood expulsion would not be uniform and instantaneous, and therefore blood may remain in the skin tissue after a laser beam has been fired. In contrast, the apparatus of the present invention is suitable for performing skin treatments when the treatment beam is 40 mm, and furthermore is suitable for performing skin treatments by means of an IPL unit having a beam diameter which is significantly larger than that of a laser unit.

g) Prior art vacuum-assisted light-based skin treatment devices are known only to reduce the concentration of blood within a skin target, in order to increase the exposure of the skin target to the treatment light. The apparatus of the present invention, however, employs a evacuation chamber overlying the skin target, as will be described hereinafter, which does not necessarily expel blood from the epidermis of the skin target, but rather increases the blood volume fraction within the skin target.

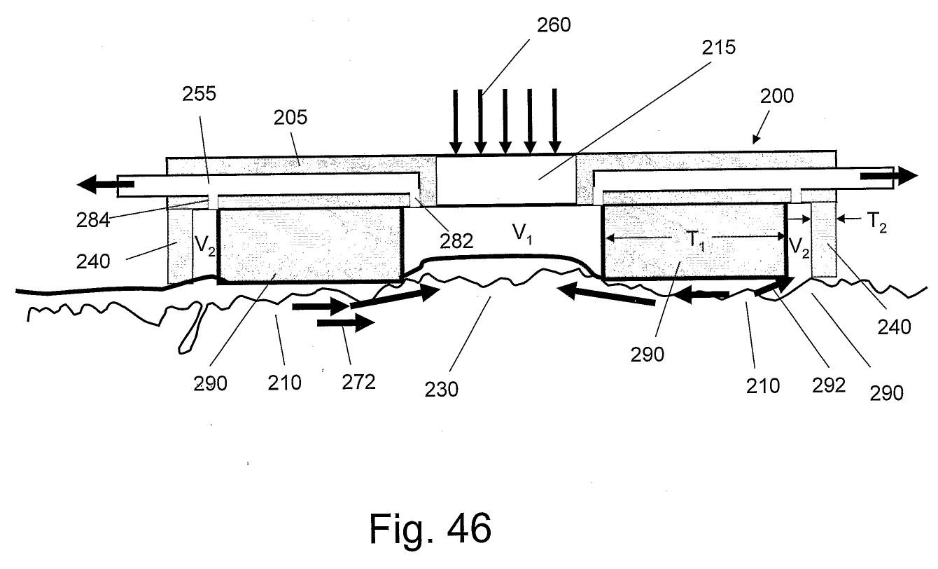

FIGS. 23 and 46 illustrate two evacuation chamber configurations, respectively, which induce different blood transfer effects. In FIG. 23, evacuation chamber 100 is configured to induce the expulsion of blood 140 from skin target 130 to peripheral skin area 135, as indicated by the direction of the arrows, while evacuation chamber 200 of FIG. 46 is configured to induce blood transfer from peripheral skin area 210 to skin target 230, as indicated by the direction of the arrows.

The direction of blood transfer is dependent on the ratio of the skin target diameter to the thickness of the evacuation chamber walls. In FIG. 23, evacuation chamber 100 has thin walls 105 which serve to squeeze blood while peripheral skin area 135 slides under walls 105 as skin target 130 is drawn proximally. As walls 105 are thinner or sharper, the localized pressure under the walls is increased, resulting in a more effective squeezing of blood in the same direction as the skin sliding direction and outwardly from walls 105. On the other hand, as shown in FIG. 46, relatively thick support elements 290 of evacuation chamber 200 induce blood transfer towards skin target 230. Due to the increased thickness of support elements 290, the frictional force applied by support elements 290 onto the underlying skin surface is increased relative to that applied by walls 105 of FIG. 23, and therefore peripheral skin area 210 is prevented from sliding under support elements 290. As support elements 290 press on the underlying skin surface, albeit by a localized pressure less than applied by walls 105 of FIG. 23, the corresponding blood vessels are squeezed and blood is forced to flow towards skin target 230.

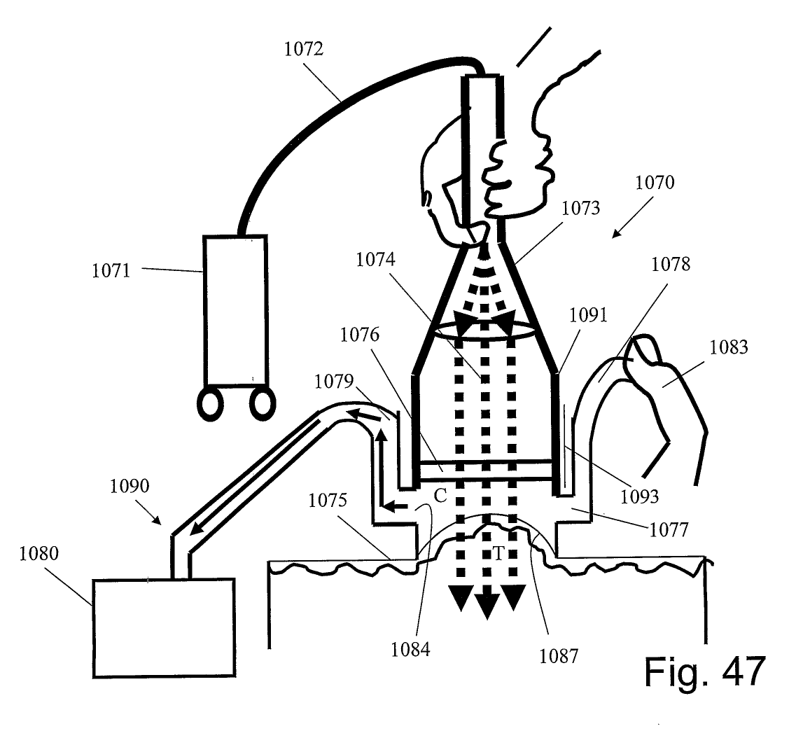

FIG. 47 illustrates the apparatus according to an embodiment of the invention, which is generally designated by numeral 1070. Apparatus 1070 comprises light source 1071, handpiece 1073 provided with transmitting element 1076 at its distal end, an evacuation unit which is designated by numeral 1090, and preferably a pressure indicator (not shown) for indicating the pressure within the evacuation chamber.

Evacuation unit 1090 comprises vacuum pump 1080, evacuation chamber C, and conduits 1078 and 1079 in communication with chamber C. Evacuation chamber C, which is placed on skin surface 1075, is formed with an aperture (not shown) on its distal end and is provided with a transmitting element 1076 on its proximate end. Evacuation chamber C is integrally formed with handpiece 1073, such that cylindrical wall 1091 is common to both handpiece 1074 and evacuation chamber C. Element 1076 is transparent to beam 1074 of intense pulsed monochromatic or non-coherent light which is directed to skin target T. Element 1076 is positioned such that beam 1074 is transmitted in a direction substantially normal to skin surface 1075 adjoining skin target T. The ratio of the maximum length to maximum width of the aperture, which may be square, rectangular, circular, or any other desired shape, ranges from approximately 1 to 4. Since the aperture is formed with such a ratio, skin target T is proximately drawn, e.g. 1 mm from skin surface 1075, and is slightly deformed, as indicated by numeral 1087, while increasing the concentration of blood in skin target T. Likewise, employment of an aperture with such a ratio precludes formation of a vacuum-induced skin fold, which has been achieved heretofore in the prior art and which would reduce the concentration of blood in skin target T.

Wall 1091 is formed with openings 1077 and 1084 in communication with conduits 1078 and 1079, respectively. The two conduits have a horizontal portion adjacent to the corresponding opening, a vertical portion, and a long discharge portion. Openings 1077 and 1084 are sealed with a corresponding sealing element 1093, to prevent seepage of fluid from the evacuation chamber. Conduit 1079 is also in communication with vacuum pump 1080, which draws fluid, e.g. air, thereto at subatmospheric pressures. U-shaped evacuation chamber C is therefore defined by transmitting element 1076 of the handpiece, slightly deformed skin surface 1087, wall 1091 and conduits 1078 and 1079.

A suitable light source is a pulsed dye laser unit, e.g. produced by Candela or Cynosure, for the treatment of vascular lesions, which emits light having a wavelength of approximately 585 nm, a pulse duration of approximately 0.5 microseconds and an energy density level of 10 J/cm². Similarly any other suitable high intensity pulsed laser unit, such as a Nd:YAG, pulsed diode, Alexandrite, Ruby or frequency doubled laser, operating in the visible or near infrared region of the spectrum may be employed. Similarly, a laser unit generating trains of pulses, such as the Cynosure Alexandrite laser, the Lumenis “Quatim” IPL or Deka “Silkapill”. The emitted light is transmitted via optical fiber 1072 to handpiece 1073. Handpiece 1073 is positioned such that transmitting element 1076 faces skin surface 1087. Beam 1074 propagating towards slightly protruded skin surface 1087 is substantially normal to skin surface 1075.

Following operation of vacuum pump 1080, air begins to become evacuated from evacuation chamber C via conduit 1079. Occluding conduit 1078, such as by placing finger 1083 of an operator on its outer opening increases the level of the vacuum within chamber C to a pressure ranging from 200 to 1000 millibar. The application of such a vacuum slightly draws skin target T towards chamber C without being pressed, as has been practiced heretofore in the prior art, thereby increased the concentration of blood vessels within skin target T. The efficacy of a laser unit in terms of treatment of vascular lesions is generally greater than that of the prior art, due to the larger concentration of blood vessels in skin target T, resulting in greater absorption of the optical energy of beam 1074 within bodily tissue.

The operator may fire the laser following application of the vacuum and the subsequent change in color of skin target T to a reddish hue, which indicates that the skin is rich in blood vessels. The time delay between the application of the vacuum and the firing of the laser is based on clinical experience or on visual inspection of the tissue color.

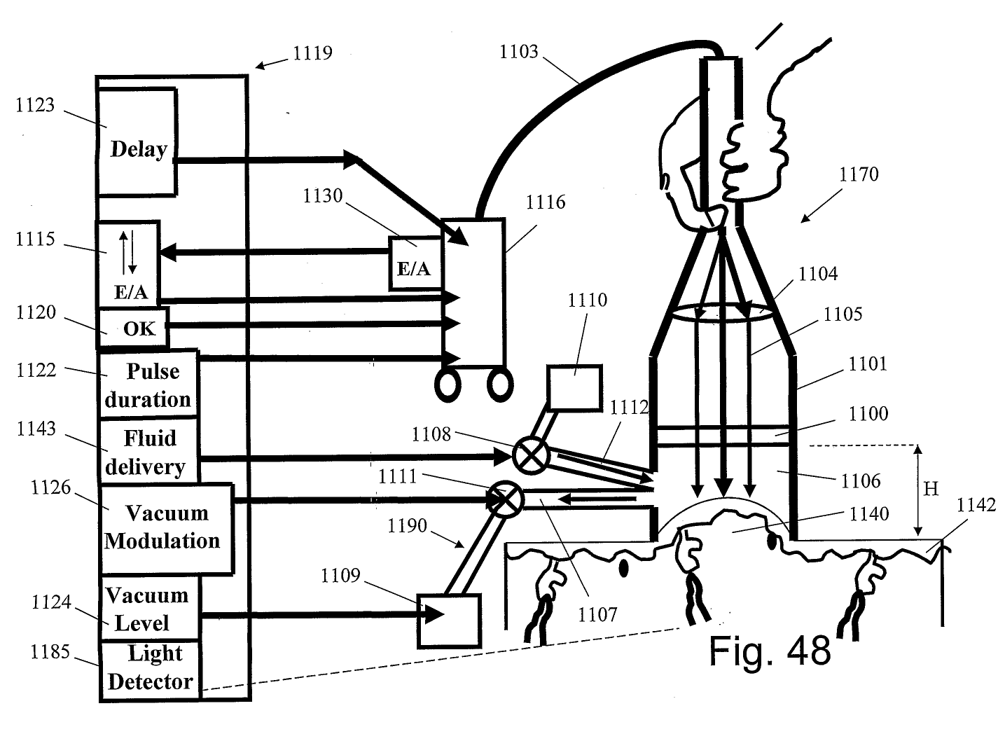

FIG. 48 illustrates another embodiment of the present invention wherein the operation of the vacuum pump and of the pulsed laser or non-coherent light source is electronically controlled. The depth of light penetration within the tissue may be controlled by controlling the time delay between application of the vacuum and the firing of the pulsed light. If the time delay is relatively short, e.g. 10 msec, blood vessel enrichment will occur only close to the surface of the skin at a depth of approximately 0.2 mm, while if the delay is approximately 300 msec, the blood vessel enrichment depth may be as great as 0.5-1.0 mm.

Apparatus 1170 comprises handpiece 1101, laser system 1116, evacuation unit 1190 and control unit 1119.

Laser system 1116 includes a power supply (not shown), a light generation unit (not shown), and power or energy detector 1130 for verifying that the predetermined energy density value is applied to the skin target. Handpiece 1101 held by the hand of the operator is provided with lens 1104, which directs monochromatic beam 1105 transmitted by optical fiber 1103 from laser system 1116 to skin target area 1140. Transmitting element 1100 defining evacuation chamber 1106 is generally in close proximity to skin surface 1142, at a typical separation H of 1-2 mm and ranging from 0.5 to 4 mm, depending on the diameter of the handpiece. The separation is sufficiently large to allow for the generation of a vacuum within chamber 1106, but less than approximately one-half the diameter of the window 1100, in order to limit the protrusion of skin target 1140 from the adjoining skin surface 1142. By limiting the separation of element 1100 from skin surface 1142 while maintaining the vacuum applied to skin target 1140, formation of a skin fold is precluded while more blood may be accumulated in a smaller skin thickness. Therefore a significant local rise in the temperature of a blood vessel, which ranges from 50-70° C., is made possible.

Evacuation unit 1190 comprises evacuation chamber 1106 which is not U-shaped, miniature vacuum pump 1109 suitable for producing a vacuum ranging from 200-1000 millibar, conduit 1107 and control valve 1111 through which subatmospheric fluid is discharged from chamber 1106, and miniature pressurized tank 1110 containing, e.g 100 ml, which delivers air through conduit 1112 and control valve 1108 to chamber 1106. If so desired, a transmitting element need not be used, and evacuation chamber 1106 defined by lens 1104 will have an accordingly larger volume.

Control unit 1119 comprises the following essential elements:

a) Display 1115 of the energy density level of the monochromatic light emitted by laser system 1116 and a selector for selecting a predetermined energy density.

b) Confirmation indicator 1120 which verifies that the selected energy density is being applied to the skin. Control circuitry deactivates the laser power supply if a beam having an energy density significantly larger than the predetermined value is being fired.

c) Display 1122 concerning the pulse structure, such as wavelength, pulse duration and number of pulses in a train.

d) Control circuitry 1123 for selecting the time delay between operation of vacuum pump 1109 and laser system 1116.

e) Selector 1124 for controlling the vacuum level in evacuation chamber 1106 by means of pump 1109.

f) Control circuitry 1126 for controlling the vacuum duty cycle by regulating the operating cycle of vacuum pump 1109, the open and close time of control valve 1111, the average vacuum pressure, the vacuum modulation frequency, and the repetition rate.

g) Control circuitry 1143 for delivering fluid from positive pressure tank 1110 by controlling the duty cycle of control valve 1108.

h) Light detector 1185 for sensing whether light is impinging onto skin target 1140.

Tank 1110, in which air having a pressure ranging from 1-2 atmospheres is contained, provides a fast delivery of less than 1 msec of air into chamber 1106, as well as a correspondingly fast regulation of the vacuum level therein by first opening control valves 1108 and 1111 and activating vacuum pump 1109. After a sufficient volume of fluid, e.g 1 ml, is delivered to chamber 1106, control valve 1108 is closed. Control circuitry 1126 and 1143 then regulate the operation of the control valves so to maintain a predetermined level of vacuum. Upon achieving the predetermined vacuum level, control circuitry 1123 fires laser system 1116 after the predetermined time delay, which may range from 1-1000 msec.

Control unit 1119 may also be adapted to increase the pressure in evacuation chamber 1106 to atmospheric pressure (hereinafter in “a vacuum release mode”) following deactivation of the pulsed light beam source, to allow for effortless repositioning of the evacuation chamber to another skin target. In order to achieve a fast response time between the deactivation of the light source and the pressure increase within the evacuation chamber prior to repositioning the evacuation chamber to another skin target, light detector 1185 is employed to detect the light emitted by the treatment light source. When the light detector ceases to detect light emitted by the light source, a suitable command is transmitted to control unit 1119, whereupon the latter generates a command to open control valve 1111, in order to increase the evacuation chamber pressure. Alternatively, the vacuum within the evacuation chamber may be released by depressing a pneumatically or electrically actuated button located on the handpiece, following deactivation of the light source. Employment of a light detector which triggers the release of the vacuum in the evacuation chamber in order to allow for the speedy repositioning of the treatment handpiece has particular significance in conjunction with fast treatment systems such as the hair removal “Light Sheer” diode system produced by Lumenis, which operates at a fast rate of 1 pulse per second.

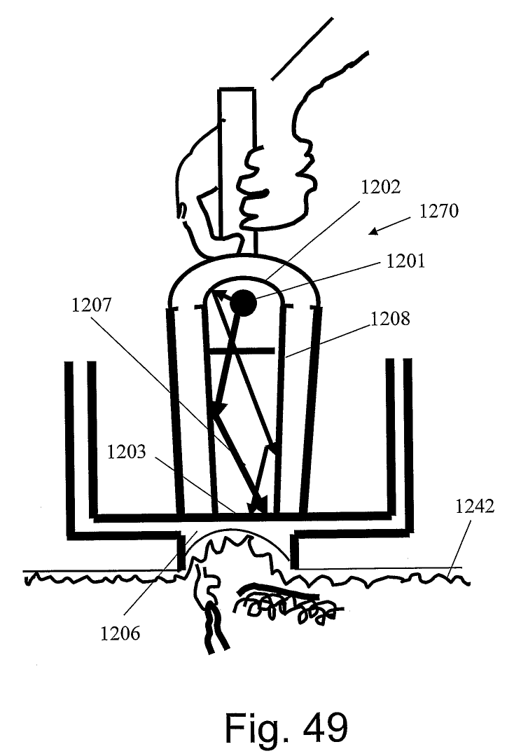

FIG. 49 illustrates apparatus 1270, which comprises a non-coherent intense pulsed light system similar to that described with respect to FIG. 42 and provided with Xe flashlamp 1201, such as one manufactured by Lumenis, Deka, Palomar, or Syneron. Reflector 1202 reflects the emitted light 1207 to light guide 1208. Distal end 1203 of light guide 1208 is separated 1-2 mm from skin surface 1242 to allow for the generation of a vacuum in evacuation chamber 1206 without compromising treatment efficacy by limiting the protrusion of the skin target from the adjoining skin surface 1242.

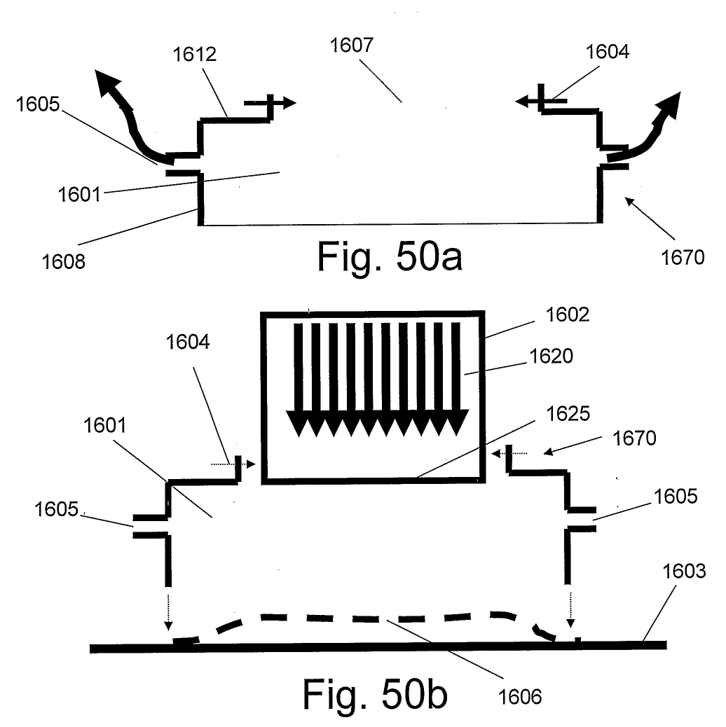

FIGS. 50a-b illustrate another embodiment of the invention wherein apparatus 1670 comprises an evacuation chamber 1601 which is attached to intense pulsed light guide 1602. FIG. 50a schematically illustrates evacuation chamber 1601 prior to attachment to the light guide, and FIG. 50b schematically illustrates the attachment of evacuation chamber 1601 to light guide 1602. Evacuation chamber 1601 has walls 1608, side openings 1605 formed in walls 1608, and proximate cover 1612 formed with a proximate aperture 1607 having dimensions substantially equal to the cross section of light guide 1602. Attachment means 1604 facilitates the attachment of evacuation chamber 1601 to light guide 1602 or to any element adapted to protect the light guide. Attachment means 1604 preferably also seals the interface between cover 1612 and light guide 1602, to prevent the infiltration of air into evacuation chamber 1601 after the generation of a vacuum therein. Transmitting element 1625 of light guide 1602 also serves to prevent an increase in evacuation chamber pressure. Once evacuation chamber 1601 is attached to light guide 1602, the evacuation chamber may be placed on a selected skin surface 1603. After a vacuum is generated within chamber 1601, skin target 1606 is drawn into the interior of evacuation chamber 1601, whereupon pulsed light beam 1620 may be fired towards skin target 1606. Evacuation chamber 1601 may be advantageously attached to the distal end of any existing IPL or laser source, to convert the light source into an apparatus for enhancing the absorption of light in targeted skin structures, in accordance with the present invention. This embodiment is particularly useful when the distal end of the light source is provided with an integral skin chilling device.

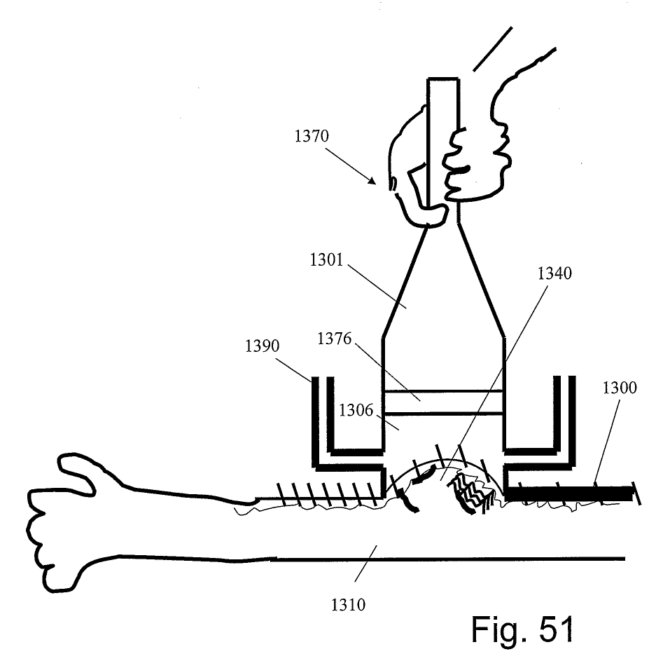

FIG. 51 illustrates the placement of apparatus 1370 onto arm 1310. Apparatus 1370 comprises handpiece 1301, evacuation unit 1390, and skin chiller 1300 for cooling the epidermis of arm 1310, which is heated as a result of the impingement of monochromatic light thereon. Skin chiller 1300 is preferably a metallic plate made of aluminum, which is in contact with the epidermis and cooled by a thermoelectric cooler. The temperature of the plate is maintained at a controlled temperature, e.g. 0° C. The chilled plate is placed on a skin region adjacent to skin target 1340. The epidermis may be chilled prior to the light treatment by other suitable means, such as by the application of a gel or a low temperature liquid or gas sprayed onto the skin target.

It will be appreciated that the utilization of a U-shaped evacuation chamber 1306 for the evacuation of vapors which condense on transmitting element 1376 is particularly advantageous when a skin chiller in permanent contact with the handpiece outer wall is employed. Such a skin chiller results in condensation of vapors on the transmitting element that would not be evacuated without employment of an evacuation unit in accordance with the present invention. Alternatively, the skin chiller may be releasably attached to the evacuation chamber.

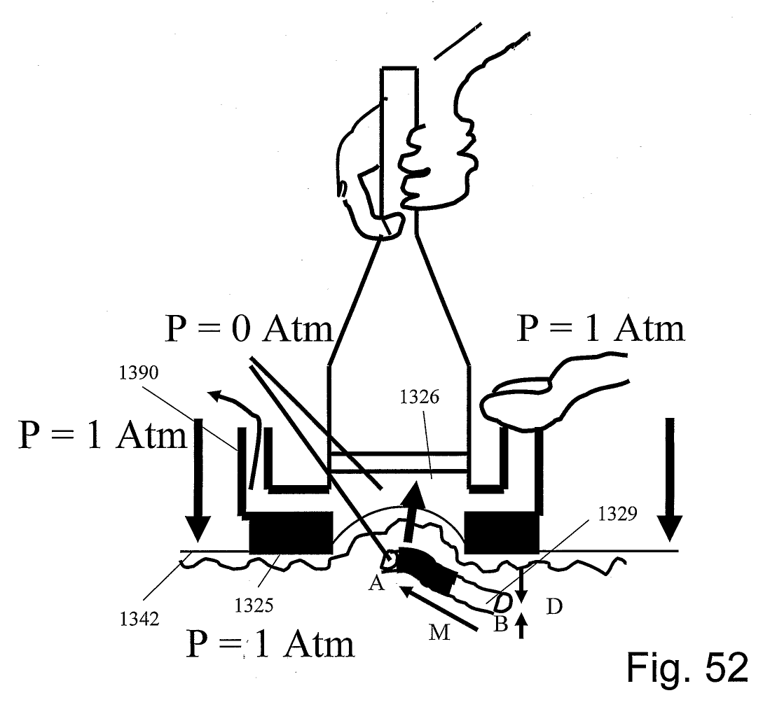

FIG. 52 schematically illustrates the effect of applying a subatmospheric pressure to a skin target, in accordance with the present invention, in order to enhance the absorption of light by blood vessels within the skin target. For clarity, the drawing illustrates the effect with respect to a single blood vessel; however, it should be appreciated that many blood vessels contribute to the effect of increased blood transport whereby a plurality of blood vessels are drawn to the epidermis, resulting in increased absorption of the optical energy. The protrusion of the skin target relative to the adjoining skin surface is also shown in disproportionate fashion for illustrative purposes.

The increase in light absorption within blood vessels due to the application of a vacuum in the vicinity of a skin target depends on the vacuum level, or the rate of vacuum modulation, and the skin elasticity which is reduced with increased age. As shown, blood vessel 1329 of diameter D is in an underlying position relative to evacuation chamber 1326. By applying a vacuum by means of evacuation unit 1390, blood flow is established in blood vessel 1329 in the direction of arrow M, due to a difference of pressures between points A and B closer and farther from evacuation chamber 1326, respectively. If the blood vessel is a vein, the flow will be established in only one direction, due to the influence of the corresponding vein valve.

According to the Hagen-Poisseuille equation concerning the flow of viscous fluids in tubes, the discharge from a tube and consequently the duration of flow therethrough depends on a pressure gradient along the tube, the fourth power of the diameter of the tube, and the length thereof. For example, diameters of 100 microns are common for capillaries adjacent to the papillary dermis at a depth of approximately 200 microns and 500-micron blood vessel diameters can be found in the hair bulb at a depth of 3 mm. A typical blood vessel length is approximately 1-2 cm. It will be appreciated that although the blood vessel diameters generally increase with depth, the pressure gradient along the blood vessel is smaller at deeper layers of the skin. As a result, for a given pressure, such as the application of a zero millibar vacuum, each depth from the skin surface corresponds to a characteristic time response for being filled by blood. As a result, modulation of the vacuum by opening and closing control valve 1111 (FIG. 48) controls the flow of blood through blood vessels and consequently controls the degree of light absorption by a blood vessel at a given depth from skin surface 1342. In a realistic situation wherein a plurality of blood vessels are located within a skin target, each skin layer is characterized by a different modulation frequency which typically ranges between 100 Hz for upper layers and 1 Hz for the deep layers under the hair follicles. By opening control valves 1108 and 1111 (FIG. 48) by a varying frequency, the operator may modulate the vacuum applied to the skin target and thereby vary the blood richness of different skin layers.

The operator typically determines an instantaneous modulation frequency of control valves 1108 and 1111 by visually inspecting the skin target and viewing the degree of redness thereat in response to a previous control valve modulation frequency. In addition to improving the treatment efficacy, an increased degree of redness within the skin target advantageously requires a lower energy density of intense pulsed light for achieving blood coagulation or blood heating resulting in the heating of the surrounding collagen. Alternatively, an errythema, i.e. skin redness, meter, e.g. produced by Courage-Hazaka, Germany, may be employed for determining the degree of redness, in order to establish the necessary energy density for the treatment.

For example, a modulation frequency as high as 40 Hz or the firing of a Dye laser unit approximately 1/40 seconds after application of a vacuum may be necessary for applications of port wine stains,. In contrast, a delay of approximately a half second for fine wrinkle removal and of approximately 1 second for hair removal may be needed for a depth of 1-3 mm under the skin surface.

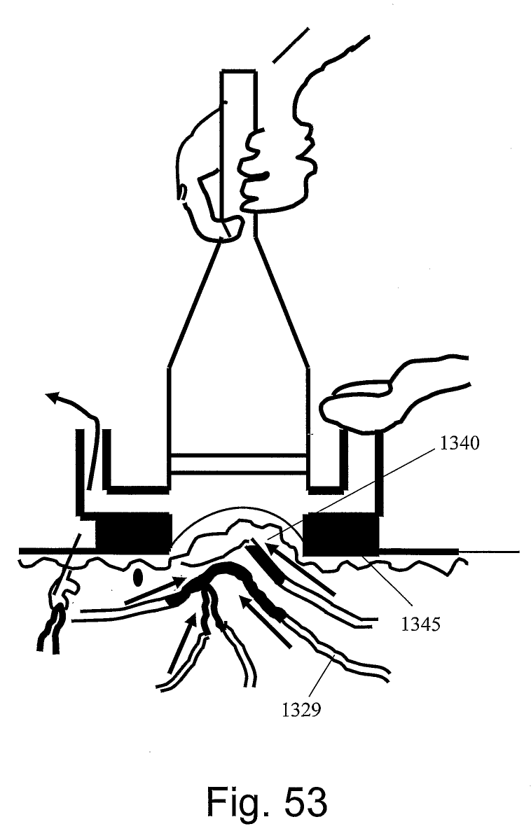

FIG. 53 illustrates the concentration of a plurality of blood vessels 1329 in a skin target 1340, which results in the increase of redness of skin and enhanced absorption of light with respect to the hemoglobin absorption spectrum and scattering properties of skin. Light absorption is enhanced by a larger number of blood vessels per unit volume due to the correspondingly larger number of light absorbing chromophores. The beneficial effect of vacuum assisted absorption by Dye lasers or any yellow light, which is strongly absorbed by hemoglobin, is more pronounced on white or yellow skin not rich in blood vessels, such as that of smokers. Such types of skin suffer from enhanced aging and require photorejuvenation, the efficacy of which is improved with the use of the present invention. Enhanced absorption of light is also advantageously achieved when infrared lasers and intense pulsed light sources are employed.

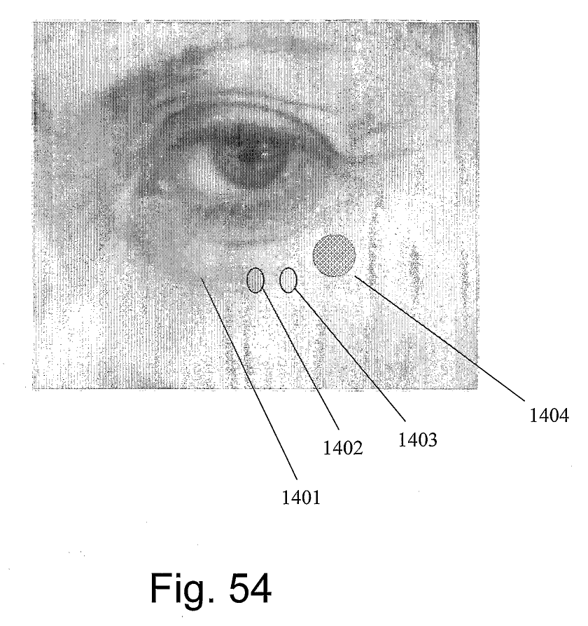

FIG. 54 is an enhanced photograph illustrating the treatment of a fine wrinkle 1401 by means of a vacuum assisted handpiece according to the current invention, which was taken one-half of a second after the application of a vacuum. Circles 1402-4 indicate the sequential change in color of the treatment spots. The color in spot 1403 has become lighter than spot 1402. Spot 1404 has become pinker than spots 1402 and 1403 due to the higher blood fraction.

FIG. 46 illustrates another embodiment of the invention, by which blood vessel concentration within a skin target is increased by selecting the thickness of the supporting elements of the evacuation chamber. Evacuation chamber 200 placed on skin target 230 comprises cover 205, transmitting element 215 centrally retained within cover 205, relatively thin annular leg 240 having a thickness of T₂ positioned below cover 205 at the outer periphery thereof, relatively thick annular support element 250 of thickness T₁ separated from leg 240 and positioned below cover 205 at skin area 210 adjoining skin target 230, and conduits 255 formed within cover 205 by which the vacuum is applied to the evacuation chamber. Each conduit 255 is provided with an inner inlet 282 and an outer inlet 284. Each inner inlet 282 communicates with volume V₁ interior to annular support element 250 and each outer inlet 284 communicates with volume V₂, which has a significantly smaller volume than volume V₁ and is formed between support element 250 and surrounding annular leg 240.

When a vacuum is applied to evacuation chamber 200, the pressure differential between the surrounding ambient air pressure and the generated vacuum within the evacuation chamber urges evacuation chamber 200 to be in pressing relation with the skin adjoining skin target 230. The resultant force associated with the pressure differential acts on both legs 240 and on support elements 290. Since a vacuum is applied onto the two sides of support element 290 via volumes V₁ and V₂, the resultant force transmitted to underlying skin area 210 by support element 290 produces a substantially uniform squeezing pressure. By virtue of thin vacuum volume V₂, legs 240 serve as a means to stabilize evacuation chamber 200, which is particularly useful on a skin area that is not completely planar, such as in the vicinity of a bone.

The wide area pressure applied by support element 290 onto skin area 210 directs the expelled blood towards skin target 230 as well as towards leg 240. Air evacuated from volume V₁ through inner inlets 282 causes skin target 230 to be proximally drawn and blood to be transported from peripheral skin area 210 towards skin target 230. Support element 290 therefore induces inward blood transport from peripheral skin areas 210 to skin target 230, as represented by arrow 272, resulting in a significant increase in the blood volume fraction within skin target 230. After the blood concentration within skin target 230 has sufficiently increased, light beam 260 is suitable for treating vascular lesions with a wavelength well absorbed by the blood vessels within the skin target, and therefore an energy density less than that of the prior art is fired. The depth of light absorption within skin target 230 can be controlled by changing the thickness T of support elements 290.

Air evacuated from volume V₂ through a corresponding outer inlet 284 causes skin area 290 underlying corresponding volume V₂ to be drawn drawn proximally. Skin area 290 is then pressed by the edge of support element 290 so that blood, as represented by arrow 292, is outwardly transported from support element 290 to leg 240. By inducing outward transport of blood, the blood volume fraction and therefore the depth of light absorption within skin target 230 may be further controlled.

It will be appreciated that the blood concentration within skin target 230 can be increased solely by the pressure applied by support element 290, without use of legs 240. Likewise, support elements 1325, 1345, and 1502 illustrated in FIGS. 10, 11, and 13, respectively, induce blood transport towards the skin target without need of additional legs.

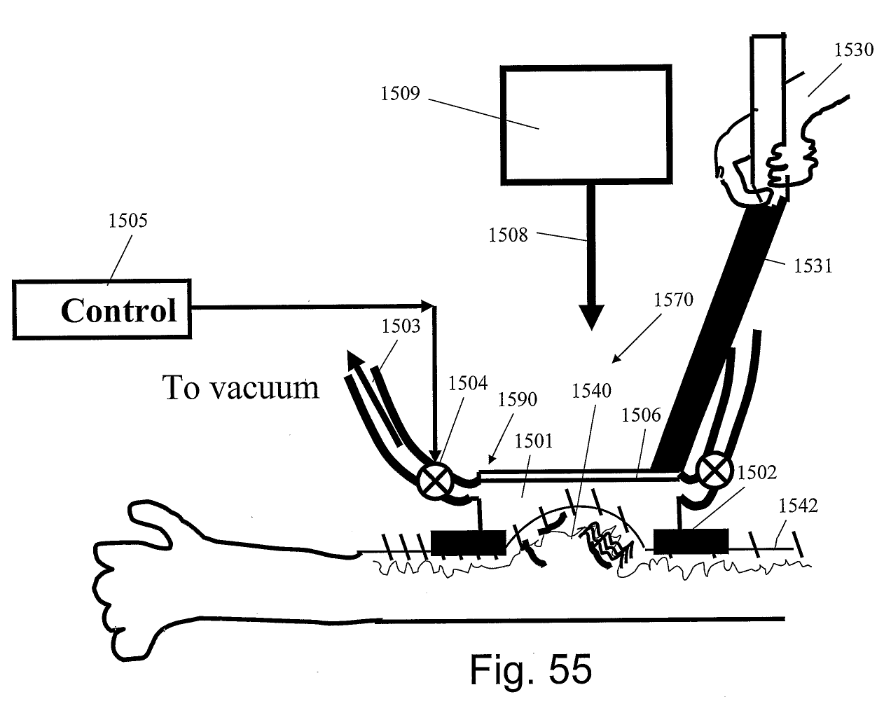

FIG. 55 illustrates apparatus 1570 which increases blood vessel concentration within a skin target without use of a handpiece. Apparatus 1570 comprises evacuation unit 1590 having a transparent evacuation chamber 1501 and a transmitting element 1506, which is made of a thin, transparent polymer such as polycarbonate or of glass, which is transparent to visible or near infrared light. Evacuation chamber 1501 has a diameter of 5-20 mm and a height of approximately 1-3 mm, in order to avoid excessive protrusion of the skin. Chamber 1501 is preferably cylindrical, although other configurations are also suitable. A soft silicon rim (not shown) is adhesively affixed to the periphery of the chamber 1501, in order to provide good contact with skin surface 1542. Conduit 1503 in communication with control valve 1504 allows for the evacuation of evacuation chamber 1501 by means of a miniature vacuum pump (not shown) and control unit 1505. After chamber 1501 is placed on skin target 1540, pulsed beam 1508 from any existing intense pulsed laser or light source 1509 which operate in the visible or near infrared regions of the spectrum may propagate therethrough and effect treatment of a skin disorder. Evacuation chamber 1501 and conduit 1503 are preferably disposable. When evacuation chamber 1501 is disposable, transmitting element 1506 is insertable within a suitable groove formed within the housing of evacuation chamber 1501. Evacuation chamber 1501 may be hand held or may be releasably attachable to the handpiece of light source 1509. When hand held, evacuation chamber 1501, control unit 1505, and a display (not shown) may be integrated into a single device. The treatment may therefore be performed with the use of two hands, one hand, e.g. hand 1530, holding the integrated evacuation chamber device by means of handle 1531 and the other holding the treatment light source. The advantage of this apparatus is its low price and its ability to interact with any intense pulsed laser or non-coherent light source which is already installed in a health clinic.

The absorption of visible intense pulsed light in blood vessels when vacuum is applied to a skin target may be enhanced by the directing electromagnetic waves to the skin target. Radio frequency waves operating in the range of 0.2-10 MHz are commonly used to coagulate tiny blood vessels. The alternating electrical field generated by a bipolar RF generator, such as produced by Elman, USA or Synron, Canada, follows the path of least electrical resistance, which corresponds to the direction of blood flow within blood vessels. A monopolar RF may also be employed, such as manufactured by Thermage, USA.

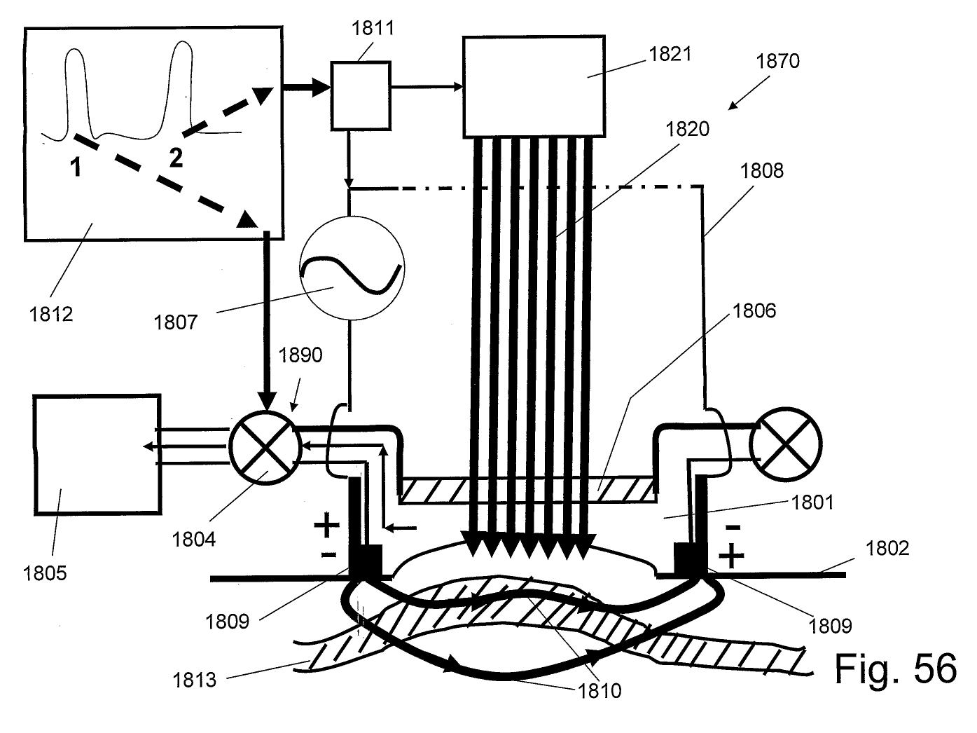

FIG. 56 illustrates apparatus 1870 which comprises intense pulsed laser or intense pulsed light source 1821, RF source 1811, and evacuation unit 1890. Evacuation unit 1890 comprises evacuation chamber 1801, which is placed on skin surface 1802 to be treated for vascular lesions, miniature vacuum pump 1805, and control valve 1804 for regulating the level of the vacuum in chamber 1801. Transmitting element 1806 is positioned in such a way that beam 1820 generated by light source 1821 propagates therethrough and impinges skin surface 1802 at an angle which is substantially normal to the skin surface.

RF source 1811 is a bipolar RF generator which generates alternating voltage 1807 applied to skin surface 1802 via wires 1808 and electrodes 1809. Alternatively, the RF source is a monopolar RF generator with a separate ground electrode. Electric field 1810 generally follows the shape of blood vessels 1813, which are the best electrical conductors in the skin. Due to the concentration of blood vessels 1813 in the epidermis, the depth of which below skin surface 1802 depending on the vacuum level and the frequency of vacuum modulation, the combined effect of optical energy in terms of beam 1820 and pulsed RF field 1810 heats or coagulates the blood vessels. Control valve 1804 is regulated by means of control unit 1812. A first command pulse 1 of control unit 1812 controls valve 1804 and a second command pulse 2 controls a delayed radio frequency pulse as well as a delayed light source pulse.

Scanning Apparatus

Some lasers for hair removal such as an Nd:YAG laser produced by Sciton Inc., USA or an Alexandrite laser produced by Lumenis employ a scanner to cover large treatment areas within a short time duration. In accordance with the present invention, a scanning laser can scan the area of a skin surface underlying the transmitting element of the evacuation chamber. Scanning is normally fast, and may reach a repetition rate of 5 pulses/sec. By employing a large transmitting element, application of the vacuum may be maintained for a sufficiently long duration to complete a full scan coverage of a treatment area. As an example, a sapphire transmitting element of 20×40 mm can be used. An Nd:YAG laser with a beam diameter of 10×10 mm will have to scan 8 spots to cover a skin area underlying the transmitting element. The scanning can be achieved within 2 seconds at a repetition rate of 4 pulses/sec. Once scanned, the vacuum is released and the process is repeated at the next skin area. Scanners may also be linear scanners which are less expansive and can utilize either a stepper motor or a galvanometric motor such as produced by Cambridge Technology, Inc., USA.

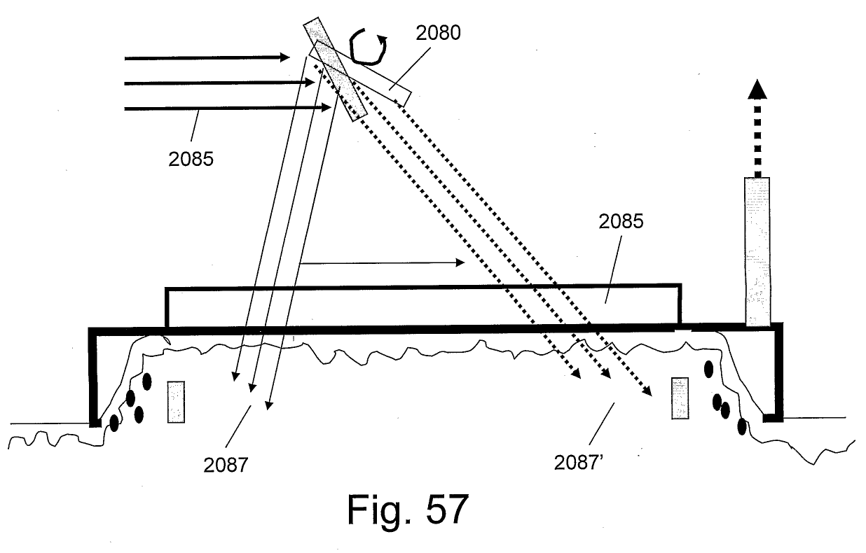

FIG. 57 schematically illustrates a pivotable linear scanner 2080 that can direct a laser beam 2085, such as generated by an Alexandrite laser, to various flattened skin targets 2087 and 2087′ underlying transmitting element 2085 of the evacuation chamber. After the entire underlying skin surface is scanned by treatment light 2085, scanner is returned to its original position and the vacuum is released, to allow the evacuation chamber to be repositioned.

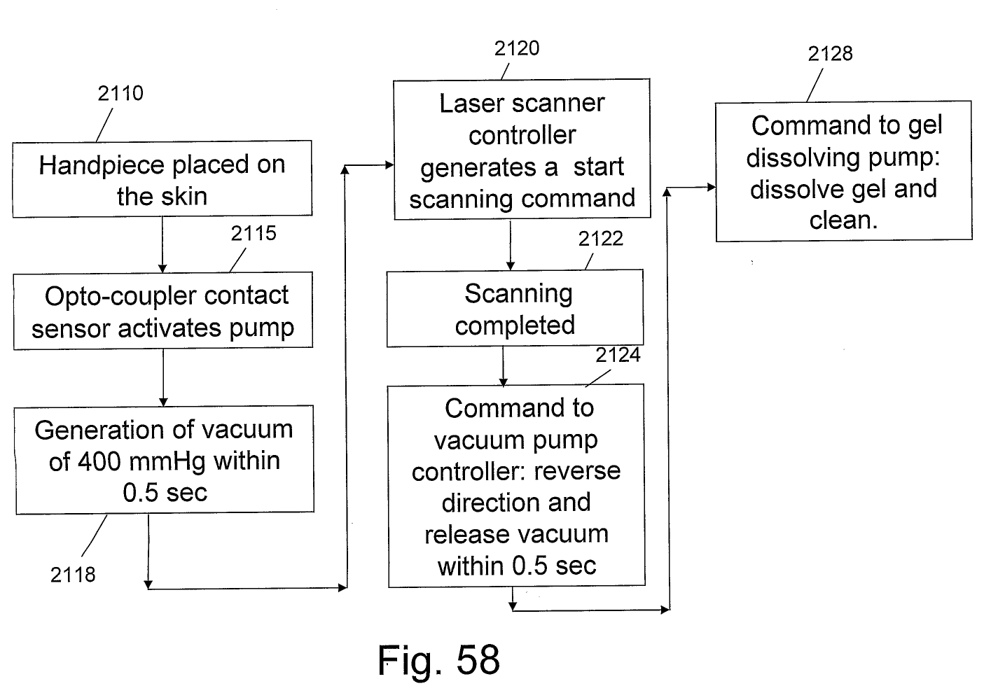

FIG. 58 illustrates a typical sequence of commands for treating a skin target with a scanner, in accordance with an embodiment of the present-invention. Such a sequence is suitable for hair removal in conjunction with an exemplary light source which is an Alexandrite laser an exemplary scanner which is a linear scanner. In step 2110, a handpiece in which the light source and evacuation chamber are housed is placed on a skin target. In step 2115, an opto-coupler contact sensor senses contact with the skin target and transmits a signal to activate the vacuum pump. In step 2118, a vacuum level of at least 400 mmHg is generated, optionally by means of a pressure sensor, within the evacuation chamber in less than 0.5 seconds. In step 2120, the laser scanner controller initiates a command to commence the scanning of a laser beam in controlled fashion throughout the entire skin surface underlying the transmitting element. After the scanning process is completed in step 2122, optionally as detected by means of an optical sensor, the vacuum pump controller is commanded in step 2124 to reverse the direction of the vacuum pump and to release the vacuum within 0.5 seconds. A gel dissolving pump is then commanded in step 2128 to deliver a dissolving solution in order to dissolve and clean gel.

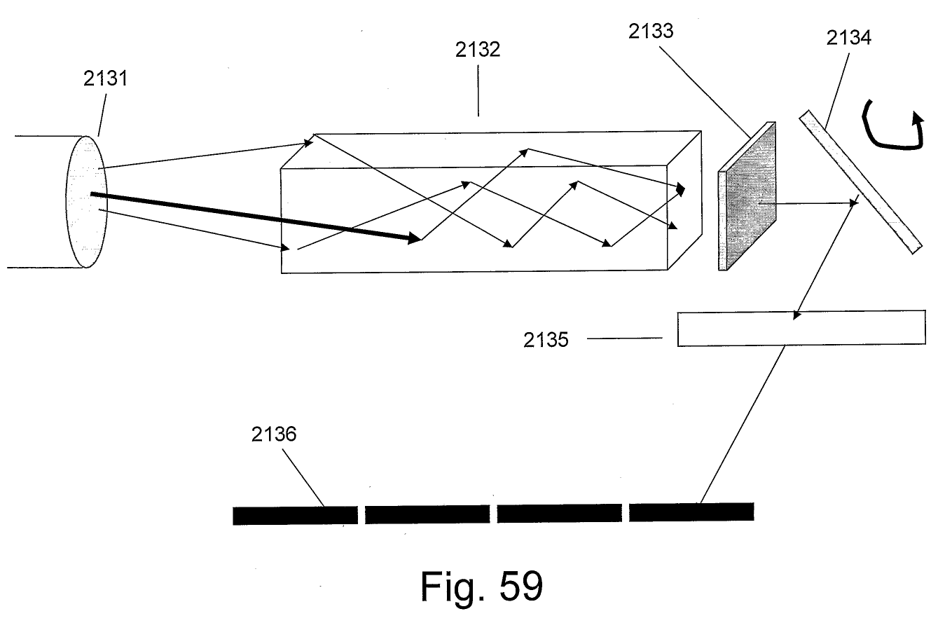

FIG. 59 illustrates another embodiment of the present invention which enables the homogeneous scanning of a laser beam such as produce by a hair removal Alexandrite laser or Nd:YAG laser on the evacuation chamber transmitting element. A distal fiber 2131 having a diameter of e.g. 1 mm produces a round beam. The output beam is fed into a square kaleidoscope 2132 having for example a width of 5 mm and a length of 50 mm. The square beam 2133 exiting kaleidoscope 2132 is imaged on the skin surface and is scanned with a scanning mirror 2134 to produce an array 2136 of square beams on the skin surface. The transformation of a round beam into a square beam enables scanning without any overlap on the evacuation chamber transmitting element. The prevention of scanning beam overlap is particularly important to avoid hyperpigmentation on dark skin.

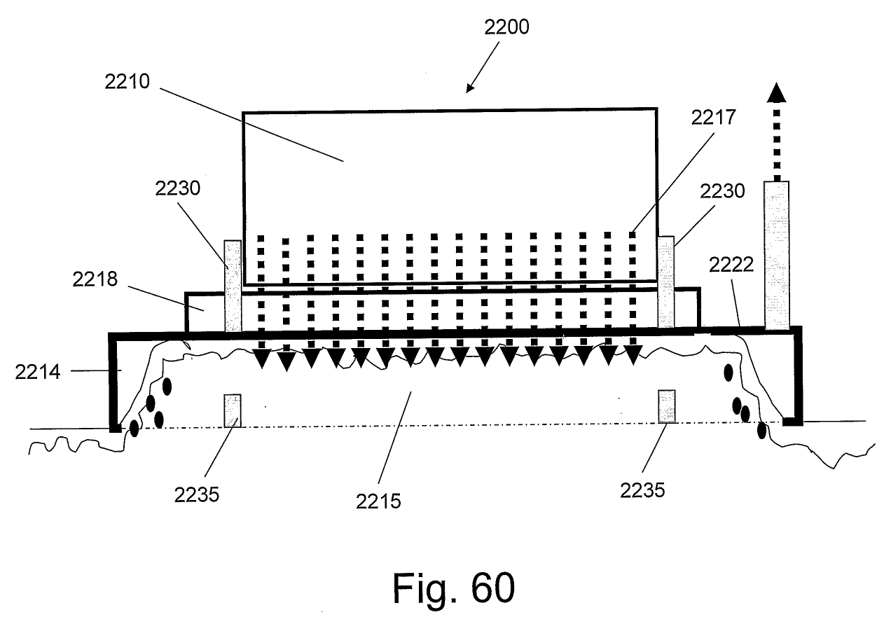

FIG. 60 illustrates apparatus 2200 which is provided with means to releasably attach the distal end of an IPL or laser source to the evacuation chamber. The releasably attaching means may be a pair of vertical walls 2230, or any other suitable mechanical elements, attached to cover 2222 of evacuation chamber 2214. Walls 2230, which may have a thickness of 2 mm and a height of 5 mm, also serve to center the distal end 2210 of an IPL source for treating skin target 2215 by treatment beam 2217 generated thereby with respect to the walls of evacuation chamber 2214, above transmitting element 2218. IPL distal end may be quickly placed the two walls 2230. Apparatus 2200 is also provided with two markers 2235 positioned on the side of evacuation chamber 2214. The spacing between the two markers 2235 is substantially equal to the diameter of beam 2217, to enable the accurate repositioning of evacuation chamber 2214 to a subsequent skin target without gaps or overlaps.

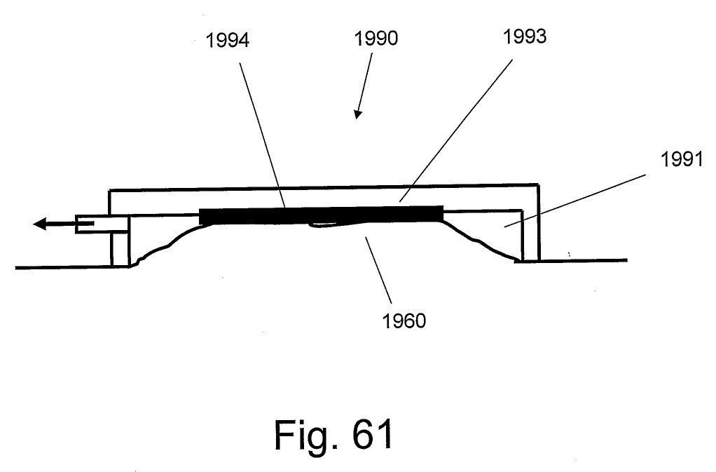

In FIG. 61, apparatus 1990 comprises a thin polycarbonate layer 1994, e.g. having a thickness of 10 microns, attached to the distal face of transmitting element 1993 and transparent to the treatment light directed to skin target 1960. Evacuation chamber 1991 is suitably sized and the applied vacuum level is sufficient to draw skin target 1960 to be in pressing contact with polycarbonate layer 1994. Polycarbonate layer 1994 is sufficiently thin to conduct heat from skin target 1960 to transmitting element 1993, is sufficiently soft to provide good mechanical matching between skin target 1960 and transmitting element 1993, and also provides good optical matching therebetween.

As described hereinabove, applying a vacuum to the evacuation chamber may either increase or decrease the blood volume fraction within a skin target, depending on a selected configuration of the evacuation chamber. Accordingly, a health professional may employ two differently configured evacuation chambers, each of which is releasably attachable to the same light source handpiece, in order to effect two distinct types of vacuum-assisted light-based treatment, respectively, with a minimum delay to the patient. Thus a single light source and a single vacuum pump may be used for both treatment of vascular lesions by increasing blood concentration within a skin target and for painless hair removal.

In summation, Table I below tabulates the main differences between prior art vacuum-assisted light-based treatment methods, by which ablated skin and vaporous debris are evacuated from a skin target, and that of the present invention:

TABLE I

| | | Prior Art |
|---|---|---|
| | Present Invention | Smoke Evacuators |
| Treatment Depth | Subcutaneous | Skin surface |
| Light source | Non-ablative, | Ablative, |
| | 400-1800 nm | above 1800 nm |
| High Vacuum Level | Yes | No; evacuated air is |
| (approximately 0.5 atm) | | replaced by fresh air |
| Automatic Release of | Yes; by means of | Not necessary due to |
| Vacuum, to Allow | control unit | low vacuum level |
| Displacement of | | |
| Treatment Handpiece | | |
| Contact between Skin | Yes; for pain | No |
| and Transmitting | alleviation | |
| element | | |
| Suitable for Employment | Yes | No |
| of Gel | | |
| Vacuum-Assisted Pain | Yes | No |
| Alleviation | | |
| Enhanced Skin Redness | Yes; when skin is | No |
| | not flattened | |
| Suitable for Non-Ablative | Yes | No; Suitable for |
| IPL and Nd:YAG, Dye, | | Ablative Laasers |
| Alexandrite, Ruby, and | | |
| Diode Lasers | | |

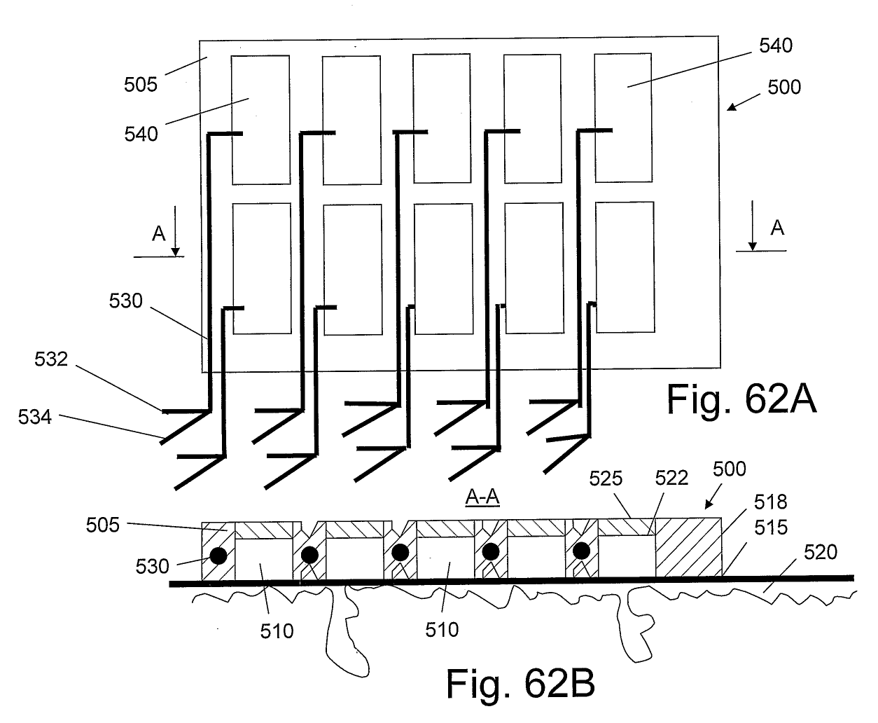

FIGS. 62A-B illustrate another embodiment of the invention by which a evacuation chamber need not be repositioned from one skin target to another. FIG. 62A is a schematic plan view of the apparatus and FIG. 62B is a cross sectional view thereof. As shown, array 500 of evacuation chambers is embodied by a single flat sheet 505, e.g. disposable and produced from low cost, transparent or translucent molded silicon, which is placed on skin surface 520 and formed with a plurality of evacuation chambers 510. The interior of each evacuation chamber 510 is defined by a bottom which is coplanar with bottom edge 515 of sheet 505, two side walls 522 extending proximally from bottom edge 515, and top edge 522 separated distally from upper surface 525 of sheet 505. A transmitting element 540 corresponding to each evacuation chamber 510 is secured to sheet 505, directly above top edge 522 of the evacuation chamber. Transmitting element 540 may be an inexpensive thin polycarbonate plate or a diffuser. The bulk material of sheet 505 is also formed with a plurality of conduits 530, each of which in communication with a corresponding evacuation chamber 510 and through which air is evacuated from the corresponding evacuation chamber. The distance between adjacent evacuation chambers 510 is sufficiently small to allow light which has diffused from the interior of each chamber to treat a skin area located underneath a corresponding conduit 530. Each conduit 530 branches into portions 532 and 534, wherein all conduit portions 532 are in communication with a vacuum pump (not shown) and all conduit portions 534 are in communication with a source of compressed air (not shown).

Array 500 advantageously allows a large-area skin surface, such as of an arm or leg, to be treated by a light source. The treatment light source is sequentially directed to each evacuation chamber 510. Following propagation of the light through a selected evacuation chamber in order to treat a corresponding skin target, the light source may be quickly moved or glided to another skin target without having to move a evacuation chamber and overcoming the force which urges it to the skin surface. Since a evacuation chamber is not displaced, gel is similarly not moved and does not accumulate. Consequently, there-is no need to provide means for preventing obstruction of gel within the vacuum pump.

Array 500 is also provided with at least one contact detector (not shown), which triggers a signal to activate the vacuum pump. When the contact detector senses the placement of array 500 on a skin surface, the vacuum pump is activated, and the air from all evacuation chambers 510 is evacuated simultaneously. The health professional then sequentially directs the light source to each evacuation chamber 510. Following completion of the treatment for the entire skin surface, the light source is deactivated and then the vacuum pump is deactivated. Alternatively, each evacuation chamber is provided with a contact detector, two control valves to control the passage of fluid through conduits portions 532 and 534, respectively, and light detector (all of which are not shown). When a treatment handpiece is placed on a transmitting element 540, the corresponding contact detector transmits a signal to activate the vacuum pump, open the control valve which regulates the fluid passage through the corresponding conduit portion 532, and then activates the light source. Upon completion of the light treatment, the light source is deactivated after a predetermined period of time or is manually deactivated. The light detector transmits a signal to close the control valve which regulates the fluid passage through the corresponding conduit portion 532 and to open the control valve which regulates the fluid passage through the corresponding conduit portion 534, in order to release the vacuum. This cycle is repeated for all evacuation chambers 510.

Vacuum-Assisted Photodynamic Therapy

The aforementioned skin flattening process can be used to improve the treatment of skin lesions with photodynamic therapy (PDT) and light which normally has a shallow penetration depth into the skin, such as blue, green or yellow light. Some lesions, such as acne rich with porphyrins, and malignant and precancerous lesions, such as actinic keratosis, can be treated by applying Levulan ALA produced by DUSA Pharmaceuticals, Inc., USA, which is absorbed by the porphyrins so as to be selectively attracted to fast dividing cells, and by photodynamic treatments. The porphyrins are selectively activated by blue light at e.g. 405 nm, by green light at e.g. 514 nm, and by yellow light at e.g. 585 nm. Melanin and blood in the skin normally do not allow light at these wavelengths to penetrate deep into the skin due to strong absorption. By stretching the skin and expelling blood from the skin which is flattened by the cover of the evacuation chamber, light penetration is enhanced and treatment is improved. An array of light emitting diodes such as produced by Philips Lumileds Lighting Company, USA having a power density of 1-20 milliwatts/cm² may be used.

In another embodiment, the transmitting element of the evacuation chamber is more separated from the skin surface, to prevent the skin target from being flattened. The applied vacuum causes emptying of the sebacious glands of acne lesions. After the vacuum is applied, blue, green or yellow treatment light may be fired, after which a skin flattening light treatment may be performed.

By employing the aforementioned skin flattening procedure, tattoos may be painlessly removed in conjunction with laser or IPL treatment light. Tattoos are often applied over large areas of the skin, such as on half the circumference of an arm, and a large number of patients are desirous of removing the tattoo after a few years. Also, eyebrow tattoos or lip tattoos fade and generally need to be removed prior to applying a new tattoo. Tattoo removal is most efficiently performed with a Q-switched laser, e.g. having an energy density of 10 J/cm² and a pulse duration of 10 nsec, with a frequency doubled Nd:YAG laser operating at 532 nm for red tattoos or having an energy density of 10 J/cm² and a pulse duration of 10 nsec for other colored tattoos, or with a Ruby, Alexandrite, or Nd:YAG laser operating at 694 nm, 755 nm, and 1064 nm, respectively, for blue tattoos, a treatment with which is often very painful when the skin target is not flattened in accordance with the method of the present invention.

Prior art wide-area tattoo removal is generally not tolerable and requires the application of a topical analgesic cream such as EMIA which is risky when applied over larger areas. By firing the tattoo removal treatment light through a transparent transmitting element of a evacuation chamber which flattens the skin at a vacuum level suitable for inhibiting pain transmission from the pain receptors in the skin target, tattoo removal from very large skin areas may be performed without any pain and without any interruptions. With use of a pain inhibiting evacuation chamber, significant pain reduction may be noticeable, such as from a pain level of 4 which is very painful to a pain level of 2 which is not painful.

When red tattoos are removed with green laser or IPL light according to prior art methods, blood vessels present in the skin are thermally damaged since red blood vessels absorb green light. The thermal damage often results in bruises which last a few days. In contrast, the skin target does not become bruised during tattoo removal in accordance with the method of the present invention due to the expulsion of blood vessels from the skin target as a result of the skin flattening process. Tattoo removal may be performed with or without the application of gel to the skin surface.

A light beam suitable for tattoo removal having a typical energy density level of 4-13 J/cm² generally does not generate an excessive amount of heat in the skin or in the transmitting element which is in contact with the flattened skin. As a result, an inexpensive glass or plastic transmitting element may be used since the use of a sapphire transmitting element having high thermal conductivity is unnecessary. Accordingly, an affordable disposable evacuation chamber for tattoo removal may be employed. Due to the superficial bleeding and the resulting skin contamination associated with tattoo removal, the use of a disposable evacuation chamber is quite beneficial. The size of a evacuation chamber for tattoo removal is selected according to the size of the tattooed area and the bodily location, e.g. an eyebrow may require a thin and elongated evacuation chamber. The typical size of a evacuation chamber ranges between 12×20 mm and 25×60 mm, although other sizes may be selected as well. A typical height of the evacuation chamber ranges between 2-8 mm.

The removal of pigmented lesions is very similar to the removal of tattoos. Tattoo removal laser and IPL units are suitable for the removal of pigmented lesions. An IPL unit is generally employed for the removal of pigmented lesions due to its capability of removing unwanted hair with the same unit. The prior art treatment of pigmented lesions is also painful, and the use of a evacuation chamber for is therefore of great utility. The size of a evacuation chamber for the treatment of pigmented lesions is similar to that for tattoo removal. An evacuation chamber which is excessively small, e.g. 5×5 mm, may not efficiently inhibit pain transmission.

### EXAMPLE 1

The pain level distribution resulting from a light-based, vacuum-assisted skin flattening skin treatment was compared to that resulting from a conventional light-based skin treatment. The light that was generated was suitable for hair removal, emitting pulses of light which were absorbed by hair follicles. The sharp burn sensation that was felt when a vacuum was not applied simulated the pain sensation which is normally associated with the injection of a needle through a skin region.

Light generated by an IPL Lovely unit manufactured by Msq Ltd., Israel and having an energy density of 18 J/cm², a wavelength greater than 640 nm, and a pulse duration of 30 msec was directed to 41 different skin targets. Light generated by an Alexandrite laser unit having an energy density of 25 J/cm² and a pulse duration of 3 msec was directed to 2 different skin targets. Light generated by a diode laser having an energy density of 42 J/cm² and a pulse duration of 2 msec was directed to 2 different skin targets. To 27 of those skin targets a vacuum of 500 mmHg was applied by means of a evacuation chamber having a planar, 20×50 mm sapphire rigid surface such that the skin target was flattened by the rigid surface. The skin treatment of the remaining 18 targets was performed without generation of a vacuum.

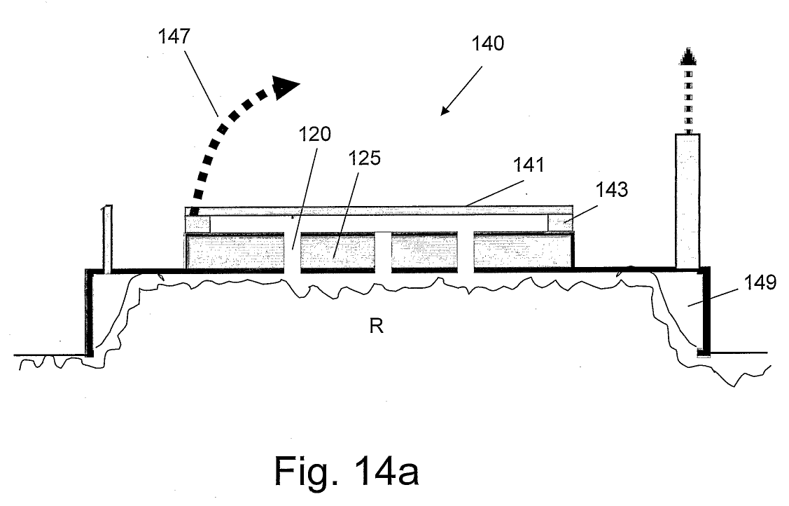

FIG. 11 illustrates a bar chart reflecting the pain sensation of patients that underwent each of the 45 skin treatments. The pain sensation was evaluated according to a modified McGill pain questionnaire and was categorized according to the Chi-square statistical technique with a deviation p of 0.06. Of the 18 skin targets that were not subjected to a vacuum, 4 (22.2%) were perceived as having a Pain Level of 5, 11 (61.1%) were perceived as having a Pain Level of 4, and 3 (16.7%) were perceived as having a Pain Level of 3. Of the 27 skin targets that were subjected to a vacuum that is capable of inducing skin flattening, 1 (3.7%) was perceived as having a Pain Level of 5, 4 (14.8%) were perceived as having a Pain Level of 4, 7 (25.9%) were perceived as having a Pain Level of 3, and 15 (55.6%) were perceived as having a Pain Level of 2. Thus the majority of targets which were not subjected to a vacuum perceived a Pain Level of 4, which is very painful, while the majority of targets that were subjected to a vacuum perceived a Pain Level of 2, which is nearly without any pain.

A patient undergoing a vacuum-assisted skin flattening skin treatment may therefore therefore anticipate a dramatic pain reduction.

### EXAMPLE 2

The influence of the vacuum level during a skin flattening skin treatment on the perceived pain level was tested. Light generated by an IPL Lovely unit manufactured by Msq Ltd., Israel and having an energy density of 18 J/cm², a wavelength greater than 640 nm, and a pulse duration of 30 msec was directed to 10 different skin targets. The pain sensation was evaluated according to a modified McGill pain questionnaire. Table I below reflects the average pain level reduction that was perceived for the different vacuum levels that were applied to each of the 10 skin targets.

At a vacuum level of approximately 150 mmHg, the perceived average pain level was 4. The perceived pain level was further reduced to a pain level of 3 when a vacuum level of 300 mmHg was applied, and a significant pain reduction to a pain level of 2 was achieved when a vacuum level of 500 mmHg was applied.

TABLE I

| Applied Vacuum (mmHg) | Level of Pain Reduction |
|---|---|
| 0 | 0 |
| 100 | 0 |
| 200 | 0 |
| 300 | 1 |
| 400 | 1 |
| 500 | 2 |

### EXAMPLE 3

The influence of the surface area of the transmitting element during a skin flattening skin treatment on the perceived pain level was tested. Light generated by an IPL Lovely unit manufactured by Msq Ltd., Israel and having an energy density of 18 J/cm², a wavelength greater than 640 nm, and a pulse duration of 30 msec was directed to 10 different skin targets. Light generated by a diode laser having an energy density of 42 J/cm² and a pulse duration of 2 msec was directed to 2 different skin targets. The vacuum level that was applied to each of the skin targets was 500 mmHg. The pain sensation was evaluated according to a modified McGill pain questionnaire.

For a rigid surface of 9×9 mm, the average perceived pain level was 3. For a rigid surface of 12×20 mm, the average perceived pain level was a tolerable 2-3. For a rigid surface of 20×40 mm, the average perceived pain level was 1-2, which was nearly without any pain.

### EXAMPLE 4

Since afferent inhibition in the dorsal horn may be limited to a few seconds, a test was conducted to determine the pain inhibiting influence of the delay between the time at which the target skin region was compressed and the time at which the target skin region was injected. Following each application of a 400 mmHg vacuum level to the evacuation chamber, a sharp needle pierced the interface element of the evacuation chamber and was pressed on the target skin region. The needle was pressed on the target skin region following various time delays ranging from 1-7 seconds after the skin region was flattened. When the delay was less than 3 seconds or greater than 6 seconds, the pain sensation was not inhibited.

### EXAMPLE 5

The capability of a planar apertured interface element for maintaining a vacuum for a sufficiently long duration within an evacuation chamber to enable the administration of painless injections, despite the presence of the apertures within the interface element, was tested. A vacuum level of 0.5 atm was applied to the evacuation chamber. The interface element, which had a length and width of 12 mm, was formed with 4 apertures having a 1-mm diameter. The upward vacuum-generated force was therefore a product of the surface area of the evacuation chamber, which was approximately 144 mm², and the vacuum level of 0.5 atm, equaling a value of 72 atm-mm². The pressure differential generated downward force applied through the apertures onto the skin on which the evacuation chamber was placed was therefore a product of the total surface area of the apertures, which was approximately 3.1 mm², and the ambient pressure level of 1 atm, equaling a value of 3.1 atm-mm², which is considerably smaller than the upward vacuum-generated force. The distance between each aperture was 8 mm, and the drawn skin region was sufficiently compressed against the solid portions of the interface element to ensure afferent inhibition. The vacuum was able to be maintained within the evacuation chamber for a duration of greater than 1 minute.

### EXAMPLE 6

The pain inhibiting capability of an evacuation chamber having an interface element with a surface area of 25×40 mm was tested. The pain sensation of two patients, to whom an injection of 1 cc of 1% lydocaine was administered, was evaluated according to a modified McGill pain questionnaire. The normal pain sensation during injection when an evacuation chamber was not employed was compared with the pain sensation resulting from an injection administered via the interface element having a surface area of 25×40 mm, when a vacuum level of 600 mmHg was generated within the evacuation chamber. A total of 10 skin regions within the two patients were injected, wherein 6 injections were vacuum assisted and 4 injections were administered when a vacuum was not generated within the evacuation chamber. With respect to all 4 injections that were not vacuum assisted, the sensed Pain Level was 4. With respect to all 6 vacuum assisted injections, the sensed Pain Level was 2. The effect of lydocaine, which is a local anesthetic typically administered prior to biopsies, was noticeable for substantially the same duration within the drawn skin region as within a non-flattened skin region, indicating that the rate of intradermal material transport was not affected by the change in volume of the drawn skin region.

### EXAMPLE 7

An experiment was performed to determine the time response of skin erythema following application of a vacuum onto various skin locations. A pipe of 6 mm diameter was sequentially placed on a hand, eye periphery, arm, and forehead at a subatmospheric pressure of approximately 100 millibar. The skin locations were selected based on the suitability for treatment: the hands and eye periphery for wrinkle removal, arm for hair removal, and forehead for port wine stain treatment. The vacuum was applied for the different periods of time of 1/10, ½, 1, 2, 3 seconds and then stopped. The erythema level and erythema delay time were then measured.

The response time of the hand and eye periphery was ½ sec, the response time of the arm was 1 second and the response time of the forehead was ½ second. Accordingly, the experimental results indicate that the necessary delay between the application of the vacuum and firing of the laser or intensed pulsed light is preferably less than 1 second, so as not to delay the total treatment time, since the repetition rate of most laser or intensed pulsed light sources is generally less than 1 pulse/sec.

The erythema delay time was less than 1 second, and therefore the experimental results indicate that patients will not sense appreciable aesthetic discomfort following treatment in accordance with the present invention.

### EXAMPLE 8

An intense pulsed light system comprising a broad band Xe flashlamp and a cutoff filter for limiting light transmission between 755 nm and 1200 nm is suitable for aesthetic treatments, such that light delivered through a rectangular light guide is emitted at an energy density of 20 J/cm² and a pulse duration of 40 milliseconds, for hair removal with respect to a treated area of 15×45 mm.

While efficacy of such a light system for the smoothening of fine wrinkles, i.e. photorejuvenation, is very limited by prior art devices, due to the poor absorption of light by blood vessels at those wavelengths, enhanced light absorption in targeted skin structures in accordance with the present invention would increase the efficacy.

A transparent evacuation chamber of 1 mm height is preferably integrally formed with a handpiece through which intense pulsed light is directed. A diaphragm miniature pump, such as one produced by Richly Tomas which applies a vacuum level of 100 millibar, is in communication with the chamber and a control valve is electronically opened or closed. When the control valve is opened, the pressure in the evacuation chamber is reduced to 100 millibar within less than 10 milliseconds. As a result of the application of vacuum, the skin slightly protrudes into the evacuation chamber at an angle as small as 1/15- 1/45 radian (height divided by size of skin target) and a height of 1 mm. Blood is drawn into the drawn skin target, which achieves a much pinker hue and therefore has a higher light absorbance. The increased redness of the skin increases the light absorption by a factor of 3. As a result, the efficacy of the aforementioned light system is similar to that of a prior art system operating at 60 Joules/cm², which is known to provide adequate results in wrinkle removal procedures. At energy density levels as high as 20 J/cm², it is preferable to chill the epidermis in order to avoid a risk of a burn. Epidermis chilling is accomplished by means of an aluminum plate, which is chilled by a thermoelectric chiller. The plate is in contact with the skin and chills the skin just before the handpiece is moved to the chilled skin target, prior to treatment.

The invention has thereby converted an intense pulsed light device for hair removal into an efficient photorejuvenation device as well.

### EXAMPLE 9

An Nd:YAG laser operating at 1064 nm, 40 milliseconds pulse duration, and energy density of 70 J/cm² is suitable for prior art hair removal having a spot size of 7 mm. By prior art hair removal, absorption of light in the hair shaft melanin is limited, with a contributory factor in hair removal being attributed to the absorption of light by blood in the hair follicle bulb zone. Since the energy density level of 70 J/cm² is risky to the epidermis of dark skin, it would be preferable to operate the laser at 40 J/cm².

A evacuation chamber is preferably integrally formed with a handpiece through which intense pulsed light is directed, at a distance of 1 mm from the skin target. A vacuum is applied to the skin target for 2 seconds. The blood concentration near the follicle bulb and in the bulge at a depth of 4 and 2 mm, respectively, is increased by a factor of 2. As a result the laser is operated with the same efficacy at energy levels closer to 40 J/cm² and is much safer.

### EXAMPLE 10

A Dye laser emitting light at a wavelength of 585 nm, with a spot size of 5 mm and pulse duration of 1 microsecond, is used by prior art methods for treatment of vascular lesions, such as telangectasia, and port wine stains, at an energy density level ranging from 10-15 J/cm² and for the smoothing of wrinkles at an energy density level of 3-4 J/cm². Some disadvantages of the prior art method are the purpura that is often produced on the skin during vascular treatments and the very large number of treatments (more than 10) which are necessary for the smoothening of wrinkles.

By applying a controlled vacuum to a evacuation chamber in contact with a skin target, having either a moderate vacuum level of approximately 600 millibars or a vacuum which is modulated at a frequency of 10 Hz for 1 seconds prior to the firing of the laser, the efficacy of the laser is enhanced. Consequently it is possible to treat vascular lesions at 7 J/cm² without creating a purpura and to remove wrinkles with a much smaller number of treatments (5).

### EXAMPLE 11

A prior art diode laser operated at 810 nm or a Dye laser is suitable for treating vascular rich psoriatic skin, wherein the treated area per pulse is approximately 1 cm². By employing a evacuation chamber attached to the distal end of the handpiece of either of these lasers, blood is drawn to the lesion and treatment efficacy is improved. The vacuum may be applied for 2 seconds prior to firing the laser beam.

### EXAMPLE 12

A deep penetrating laser, such as a pulsed diode laser at 940 nm, an Nd:YAG laser, or an intense pulsed light source operating at an energy density of 30 J/cm², is suitable for thermally damaging a gland, when a evacuation chamber is attached to the distal end of the handpiece thereof. When vacuum is applied for a few seconds, e.g. 1-10 seconds, above a gland such as a sweat gland, excessive blood is drawn into the gland. After the pulsed laser beam is directed to the skin, the absorption of the laser beam by the drawn blood generates heat in the gland, which is thereby damaged. It is therefore possible to more efficiently thermally damage glands with a laser or intense pulsed light source when vacuum is applied to the skin.

### EXAMPLE 13

By placing a evacuation chamber on a skin target in accordance with the present invention prior to the firing of an intense pulsed light source, the treatment energy density level for various types of treatment is significantly reduced with respect with that associated with prior art devices. The treatment energy density level is defined herein as the minimum energy density level which creates a desired change in the skin structure, such as coagulation of a blood vessel, denaturation of a collagen bundle, destruction of cells in a gland, destruction of cells in a hair follicle, or any other desired effects.

The following is the treatment energy density level for various types of treatment performed with use of the present invention and with use of prior art devices:

a) treatment of vascular lesions, port wine stains, telangectasia, rosacea, and spider veins with light emitted from a dye laser unit and having a wavelength of 585 nm: 5-12 J/cm² (present invention), 10-15 J/cm² (prior art);

b) treatment of vascular lesions, port wine stains, telangectasia, rosacea, and spider veins with light emitted from a diode laser unit and having a wavelength of 940 nm: 10-30 J/cm² (present invention), 30-40 J/cm² (prior art);

c) treatment of vascular lesions with light emitted from an intense pulsed non-coherent light unit and having a wavelength of 570-900 nm: 5-20 J/cm² (present invention), 12-30 J/cm² (prior art);

d) treatment of vascular lesions with light emitted from a KPP laser unit manufactured by Laserscope, USA, and having a wavelength of 532 nm: 4-8 J/cm² (present invention), 8-16 J/cm² (prior art);

e) photorejuvination with light emitted from a dye laser unit and having a wavelength of 585 nm: 2-4 J/cm² and requiring 6 treatments (present invention), 2-4 J/cm² and requiring 12 treatments (prior art);

f) photorejuvination with light emitted from a an intense pulsed non-coherent light unit and having a wavelength ranging from 570-900 nm: 5-20 J/cm² (present invention), approximately 30 J/cm² (prior art);

g) photorejuvination with a combined effect of light emitted from an intense pulsed non-coherent light unit and having a wavelength ranging from 570-900 nm and of a RF source: 10 J/cm² for both the intense pulsed non-coherent light unit and RF source (present invention), 20 J/cm² for both the intense pulsed non-coherent light unit and RF source (prior art);

h) hair removal with light emitted from a Nd:YAG laser unit and having a wavelength of 1604 nm: 25-35 J/cm² (present invention), 50-70 J/cm² (prior art);

i) porphyrin-based photodynamic therapy with light emitting diodes delivering blue light (420 nm), orange light (585 nm), or red light (630 nm) for a treatment duration ranging from 10 msec to 10 min: 5-20 J/cm² (present invention), 20-30 J/cm² (prior art).

### EXAMPLE 14

A evacuation chamber made of polycarbonate having a length of 50 mm, a width of 25 mm, a height of 3 mm, and a transmitting element made of sapphire was used during the treatment of unwanted hairs of 5 patients with an intense pulsed light system which emitted energy in the spectral band of 670-900 nm. A thin layer of gel at room temperature having a thickness of 0.5 mm was applied to a skin target. The suction openings had a diameter of 1 mm and were formed in the evacuation chamber walls at a height of 0.5 mm below the transmitting element, in order to prevent the obstruction of the openings by gel or by the drawn skin. A small canister serving as a gel trap was provided intermediate to the fluid passage between the evacuation chamber and the vacuum pump, to prevent gel from being drawn to the inlet port of the vacuum pump. A vacuum level of 500 mmHg was generated within the evacuation chamber and caused the skin target to be drawn in contact with the transmitting element.

An intense pulsed light system having a treatment beam length of 40 mm and width of 15 mm was fired with an energy density of 16-20 J/cm² and a pulse duration of 30-40 milliseconds. One patient underwent a back hair removal treatment, wherein areas of the back were treated as a control without application of a vacuum onto the skin surface and other areas were treated while a vacuum was applied to the skin surface. The other patients underwent a hair removal treatment on their legs, chest and abdomen such that a vacuum was applied to some areas, while the treatment of an adjacent area was not vacuum assisted, as a control. For all five patients, a skin chiller was not employed.

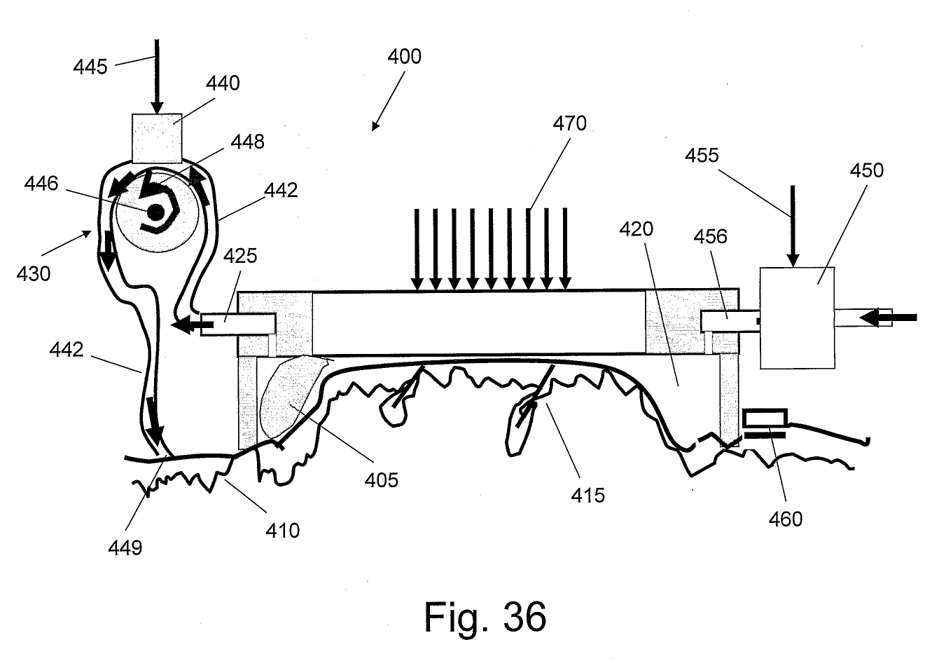

FIG. 35 is a photograph which illustrates two back areas 1985 and 1986, respectively, of one of the patients two months after being treated for hair removal. A vacuum was not applied to the skin surface of back area 1985, while a vacuum was applied to the skin surface of back area 1986. As shown, both back areas remained hairless two months after treatment.

The pain sensation of the patients was categorized into five levels: Level 0 indicating that pain was not felt at all, Level 5 indicating that pain was untolerable after a few laser shots whereby a patient grimaced and uncontrollably reacted after each shot, Level 1 indicating that the treatment was sensed but without pain, and Levels 2, 3, and 4 indicating an increasing level of pain. All of the patients consistently suffered Pain Level 3-5 when a vacuum was not applied, and the pain was alleviated (Level 2) or was completely prevented (Level 1 or 0) when a vacuum was applied. Pain alleviation was found to be dependent on the time delay between the application of the vacuum and the firing of the intense pulsed light. Pain alleviation was sensed when the intense pulsed light was fired at least 1.5 seconds after application of the vacuum onto the skin surface.

### EXAMPLE 15

A patient undergoing a hair removal treatment was tested for pain sensitivity. An intense pulsed Diode laser (Light Sheer, Lumenis) operating at 810 nm was employed. A evacuation chamber made of polycarbonate having a length of 40 mm, a width of 15 mm, a height of 3 mm, and a transmitting element made of sapphire was used. A thin layer of gel at room temperature having a thickness of 0.5 mm was applied to a skin target. The suction openings had a diameter of 1 mm and were formed in the evacuation chamber walls at a height of 0.5 mm below the transmitting element. A small canister serving as a gel trap was provided intermediate to the fluid passage between the evacuation chamber and the vacuum pump, to prevent gel from being drawn to the inlet port of the vacuum pump.

When a vacuum was not applied to the skin target and the light source operated at an energy density of 42 J/cm² and a pulse duration of 30 milliseconds, the patient sensed a Pain Level of 5. When a vacuum level of 500 mmHg was generated within the evacuation chamber causing the skin target to be drawn in contact with the transmitting element and the light source operated at an energy density of 42 J/cm² and a pulse duration of 30 milliseconds, the patient sensed a considerably reduced Pain Level of 2. This reduced pain level during the vacuum assisted treatment was found to be equivalent to the mild pain sensed when the light source operated at an energy density of only 26 J/cm² and a pulse duration of 30 milliseconds and a vacuum was not applied to the skin target.

### EXAMPLE 16

The casing of a tested Wankel type vacuum pump in accordance with the present invention had a width of 50 mm, a length of 50 mm, and a height of 10 mm. The length of the central face slots was 20 mm. The rotational speed of the pump rotor was 1500 rpm, or 25 revolutions per second, which was achieved by means of a small brushless motor. At such a rotational speed, the evacuation rate was 18 cm³/sec for an average volume of a vacuum generating compartment of 0.25 cm³. This evacuation rate is suitable for evacuating a evacuation chamber having typical dimensions of 20 mm×40 mm×5 mm height, or a typical volume of 4 cm³, within approximately 0.2 seconds. Since the vacuum needs to be generated prior to the firing of a light-based treatment pulse, the treatment speed was able to exceed a rate of 1 Hz. For a 500-pulse treatment and an average vacuum generation duration of 1 second for each treatment pulse, 12,500 rotor revolutions are needed. Plastic materials with a low friction coefficient of e.g. 0.1 wear only after approximately 50,000 revolutions, and therefore the pump is certainly durable for a 500-pulse treatment.

While some embodiments of the invention have been described by way of illustration, it will be apparent that the invention can be carried into practice with many modifications, variations and adaptations, and with the use of numerous equivalents or alternative solutions that are within the scope of persons skilled in the art, without departing from the spirit of the invention or exceeding the scope of the claims.

1-131. (canceled)

132. An apparatus (20, 30, 90, 100, 120, 130, 140, 150, 160, 219, 250, 700, 775, 1970) which is adapted to inhibit pain signals generated by pain receptors in the skin during a skin related medical treatment from being transmitted to the brain by inducing a controlled compression of a skin region, comprising:
a) an evacuation chamber (19, 12, 115) comprising an essentially rigid interface element (22, 32) through which a medical treatment can be administered to a selected skin region (26) one or more walls which are placeable on, or in the vicinity of, said skin region, an interior defined by at least said one or more walls and by said interface element, and an opening at the bottom of said interior which is sealable by said skin region;
b) means for generating a vacuum (28, 112) within said evacuation chamber interior, the level of the generated vacuum being suitable for drawing said skin region through said opening towards said interface element; and
c) means (25, 31, 184, 783, 1908) for administering a painful skin related medical treatment, said administering means adapted to pass through said interface element and to be directed to said drawn skin region,
characterized in that the surface area of said interface element is greater than a threshold surface area and in that the vacuum level within the evacuation chamber is greater than a threshold vacuum level of at least approximately 400 mmHg which is suitable for drawing said skin region in a compressing relation against said interface element,
said threshold vacuum level and said threshold surface area in combination being suitable for inhibiting the transmission of a pain signal generated by pain receptors located within said compressed skin region during said medical treatment.

133. A method for inhibiting pain signals generated by pain receptors in the skin during a skin related medical treatment, comprising the steps of:
a) positioning a rigid interface element above a selected skin region;
b) applying a vacuum of a sufficient value over said skin region such that the latter is flattened and compressed against said interface element; and
c) administering a skin related medical treatment by means adapted to pass through said interface element and to be directed to said compressed skin target, whereby pain signals generated by the nervous system during said medical treatment are inhibited due to the contact of said skin region onto said interface element.

134. An apparatus for vacuum-assisted light-based skin treatments, comprising:
a) a non-ablative intense pulsed monochromatic or non-coherent light source;
b) a vacuum chamber placeable on a skin target which has an opening on the distal end thereof and provided with a transmitting element on the proximate end thereof, said transmitting element being transparent or translucent to light generated by said source and directed to said skin target;
c) means for applying a vacuum to said vacuum chamber, the level of the applied vacuum suitable for drawing said skin target to said vacuum chamber via said opening; and
d) means for preventing influx of air into vacuum chamber during a vacuum applying mode.

135. An apparatus for controlling the depth of light absorption by blood vessels under a skin surface, comprising:
a) a vacuum chamber placed on a skin target which is formed with an aperture on the distal end thereof and provided with a transmitting element on the proximate end thereof, said transmitting element being transparent or translucent to intense pulsed monochromatic or non-coherent light directed to said skin target and suitable for transmitting the light in a direction substantially normal to a skin surface adjoining said skin target;
b) means for applying a vacuum to said vacuum chamber, the level of the applied vacuum suitable for drawing said skin target to said vacuum chamber via said aperture; and
c) means for inducing an increase or decrease in the concentration of blood and/or blood vessels within a predetermined depth below the skin surface of said skin target, and optical energy associated with the directed light being absorbed within said predetermined depth and suitable for thermally injuring or treating predetermined skin structures located at said depth.

136. Use of a peristaltic pump to generate a vacuum in a vacuum chamber placed on a gel coated skin area.

137. An apparatus for the treatment of skin disorders, comprising:
a) a vacuum chamber placeable on a skin target which has an opening on the distal end thereof and provided with a transmitting element on the proximate end thereof;
b) means for applying a vacuum to said vacuum chamber, the level of the applied vacuum suitable for drawing said skin target to said vacuum chamber via said opening and for inducing an increase in the concentration of blood and/or blood vessels below the skin surface of said skin target; and
c) a light source suitable for emitting light which is transmitted through said vacuum chamber and propagates through said skin target, and for treating a skin disorder present on said skin target.

138. An apparatus for the treatment of skin disorders, comprising:
a) a vacuum chamber placeable on a skin target which has an opening on the distal end thereof and provided with a transmitting element on the proximate end thereof;
b) means for applying a vacuum to said vacuum chamber, the level of the applied vacuum suitable for drawing said skin target to said vacuum chamber via said opening and for inducing a change in spectral properties of said skin target; and
c) a light source suitable for emitting light which is transmitted through said vacuum chamber and propagates through said skin target, and for treating a skin disorder present on said skin target.
